SEQUENCE LISTING (43 sequences)

SEQ ID NO: 1 - DNA, 6496 bp, Artificial Sequence
  Synthetic construct
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctagcgaagttcctattccgaagttcctattctctagaaagtataggaacttcagatccaccggctagaggatccaccatggttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggatccaccatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgggtaccgagctcgaattcggcccaagtttgtacaaaaaagcaggctccggccagaatggcccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaccggtcgccaccatggccctgtccaacaagttcatcggcgacgacatgaagatgacctaccacatggacggctgcgtgaacggccactacttcaccgtgaagggcgagggcagcggcaagccctacgagggcacccagacctccaccttcaaggtgaccatggccaacggcggccccctggccttctccttcgacatcctgtccaccgtgttcatgtacggcaaccgctgcttcaccgcctaccccaccagcatgcccgactacttcaagcaggccttccccgacggcatgtcctacgagagaaccttcacctacgaggacggcggcgtggccaccgccagctgggagatcagcctgaagggcaactgcttcgagcacaagtccaccttccacggcgtgaacttccccgccgacggccccgtgatggccaagaagaccaccggctgggacccctccttcgagaagatgaccgtgtgcgacggcatcttgaagggcgacgtgaccgccttcctgatgctgcagggcggcggcaactacagatgccagttccacacctcctacaagaccaagaagcccgtgaccatgccccccaaccacgtggtggagcaccgcatcgccagaaccgacctggacaagggcggcaacagcgtgcagctgaccgagcacgccgtggcccacatcacctccgtggtgcccttctgaagcggccgcaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgggcctagaaggccgatctcccgggcacccagctttcttgtacaaagtggccgttaacggatcccggtgaagttcctattccgaagttcctattctccagaaagtataggaacttc

SEQ ID NO: 2 - DNA, 1991 bp, Zea mays
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 3 - DNA, 678 bp, unknown
  Discosoma sp
atggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtag

SEQ ID NO: 4 - protein, 225 aa, unknown
  Discosoma sp
MASSENVITEFMRFKVRMEGTVNGHEFEIEGEGEGRPYEGHNTVKLKVTKGGPLPFAWDILSPQFQYGSKVYVKHPADIPDYKKLSFPEGFKWERVMNFEDGGVATVTQDSSLQDGCFIYKVKFIGVNFPSDGPVMQKKTMGWEASTERLYPRDGVLKGETHKALKLKDGGHYLVEFKSIYMAKKPVQLPGYYYVDAKLDITSHNEDYTIVEQYERTEGRHHLFL

SEQ ID NO: 5 - DNA, 696 bp, unknown
  Zoanthus sp
atggcccacagcaagcacggcctgaaggaggagatgaccatgaagtaccacatggagggctgcgtgaacggccacaagttcgtgatcaccggcgagggcatcggctaccccttcaagggcaagcagaccatcaacctgtgcgtgatcgagggcggccccctgcccttcagcgaggacatcctgagcgccggcttcaagtacggcgaccggatcttcaccgagtacccccaggacatcgtggactacttcaagaacagctgccccgccggctacacctggggccggagcttcctgttcgaggacggcgccgtgtgcatctgtaacgtggacatcaccgtgagcgtgaaggagaactgcatctaccacaagagcatcttcaacggcgtgaacttccccgccgacggccccgtgatgaagaagatgaccaccaactgggaggccagctgcgagaagatcatgcccgtgcctaagcagggcatcctgaagggcgacgtgagcatgtacctgctgctgaaggacggcggccggtaccggtgccagttcgacaccgtgtacaaggccaagagcgtgcccagcaagatgcccgagtggcacttcatccagcacaagctgctgcgggaggaccggagcgacgccaagaaccagaagtggcagctgaccgagcacgccatcgccttccccagcgccctggcctga

SEQ ID NO: 6 - protein, 231 aa, unknown
  Zoanthus sp
MAHSKHGLKEEMTMKYHMEGCVNGHKFVITGEGIGYPFKGKQTINLCVIEGGPLPFSEDILSAGFKYGDRIFTEYPQDIVDYFKNSCPAGYTWGRSFLFEDGAVCICNVDITVSVKENCIYHKSIFNGVNFPADGPVMKKMTTNWEASCEKIMPVPKQGILKGDVSMYLLLKDGGRYRCQFDTVYKAKSVPSKMPEWHFIQHKLLREDRSDAKNQKWQLTEHAIAFPSALA

SEQ ID NO: 7 - DNA, 48 bp, Saccharomyces cerevisiae
gaagttcctattccgaagttcctattctctagaaagtataggaacttc

SEQ ID NO: 8 - DNA, 48 bp, Saccharomyces cerevisiae
gaagttcctattccgaagttcctattctccagaaagtataggaacttc

SEQ ID NO: 9 - DNA, 48 bp, Saccharomyces cerevisiae
gaagttcctattccgaagttcctattctacatagagtataggaacttc

SEQ ID NO: 10 - DNA, 634 bp, Saccharomyces cerevisiae
atgccccagttcgacatcctctgcaagaccccccccaaggtgctcgtgaggcagttcgtggagaggttcgagaggccctccggcgagaagatcgccctctgcgccgccgagctcacctacctctgctggatgatcacccacaacggcaccgccattaagagggccaccttcatgtcatacaacaccatcatctccaactccctctccttcgacatcgtgaacaagtccctccagttcaaatacaagacccagaaggccaccatcctcgaggcctccctcaagaagctcatccccgcctgggagttcaccatcatcccctactacggccagaagcaccagtccgacatcaccgacatcgtgtcatccctccagcttcagttcgagtcctccgaggaggctgacaagggcaactcccactccaagaagatgctgaaggccctcctctccgagggcgagtccatctgggagatcaccgagaagatcctcaactccttcgagtacacctccaggttcactaagaccaagaccctctaccagttcctcttcctcgccaccttcatcaactgcggcaggttctcagacatcaagaacgtggaccccaagtccttcaagctcgtgcagaacaagtacctag

SEQ ID NO: 11 - DNA, 637 bp, Saccharomyces cerevisiae
tgtcatcatccagtgcctcgtgaccgagaccaagacctccgtgtccaggcacatctacttcttctccgctcgcggcaggatcgaccccctcgtgtacctcgacgagttcctcaggaactcagagcccgtgctcaagagggtgaacaggaccggcaactcctcctccaacaagcaggagtaccagctcctcaaggacaacctcgtgaggtcctacaacaaggccctcaagaagaacgccccctactccatcttcgccatcaagaacggccccaagtcccacatcggtaggcacctcatgacctccttcctctcaatgaagggcctcaccgagctcaccaacgtggtgggcaactggtccgacaagagggcctccgccgtggccaggaccacctacacccaccagatcaccgccatccccgaccactacttcgccctcgtgtcaaggtactacgcctacgaccccatctccaaggagatgatcgccctcaaggacgagactaaccccatcgaggagtggcagcacatcgagcagctcaagggctccgccgagggctccatcaggtaccccgcctggaacggcatcatctcccaggaggtgctcgactacctctcctcctacatcaacaggaggatctga

SEQ ID NO: 12 - DNA, 9982 bp, Artificial Sequence
  Synthetic construct
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttactaccggttcactagctagctgctaaggttaccagagctggtcacctttgtccaccaacttattaagtatctagttgaagacacgttcttcttcacgtaagaagacactcagtagtcttcggccagaatggcctcttgattcagcgggcctagaaggccggatcactgactagctaatttaaatcctgaggatatcgctatcaactttgtatagaaaagttgaagcttcgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctgctagcgaagttcctattccgaagttcctattctctagaaagtataggaacttcagatcctagaggatcgatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagatccatgcatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgggtaccgagctcgaattcggcccaagtttgtacaaaaaagcaggctccggccagagttacccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggccgaagctaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgggtcacccggtccgggcctagaaggccgatctcccgggcacccagctttcttgtacaaagtggccgttaacggatcccggtgaagttcctattccgaagttcctattctccagaaagtataggaacttcactagagcttgcggccgccccggagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaacaatgccccagttcgacatcctctgcaagaccccccccaaggtgctcgtgaggcagttcgtggagaggttcgagaggccctccggcgagaagatcgccctctgcgccgccgagctcacctacctctgctggatgatcacccacaacggcaccgccattaagagggccaccttcatgtcatacaacaccatcatctccaactccctctccttcgacatcgtgaacaagtccctccagttcaaatacaagacccagaaggccaccatcctcgaggcctccctcaagaagctcatccccgcctgggagttcaccatcatcccctactacggccagaagcaccagtccgacatcaccgacatcgtgtcatccctccagcttcagttcgagtcctccgaggaggctgacaagggcaactcccactccaagaagatgctgaaggccctcctctccgagggcgagtccatctgggagatcaccgagaagatcctcaactccttcgagtacacctccaggttcactaagaccaagaccctctaccagttcctcttcctcgccaccttcatcaactgcggcaggttctcagacatcaagaacgtggaccccaagtccttcaagctcgtgcagaacaagtacctaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtgtcatcatccagtgcctcgtgaccgagaccaagacctccgtgtccaggcacatctacttcttctccgctcgcggcaggatcgaccccctcgtgtacctcgacgagttcctcaggaactcagagcccgtgctcaagagggtgaacaggaccggcaactcctcctccaacaagcaggagtaccagctcctcaaggacaacctcgtgaggtcctacaacaaggccctcaagaagaacgccccctactccatcttcgccatcaagaacggccccaagtcccacatcggtaggcacctcatgacctccttcctctcaatgaagggcctcaccgagctcaccaacgtggtgggcaactggtccgacaagagggcctccgccgtggccaggaccacctacacccaccagatcaccgccatccccgaccactacttcgccctcgtgtcaaggtactacgcctacgaccccatctccaaggagatgatcgccctcaaggacgagactaaccccatcgaggagtggcagcacatcgagcagctcaagggctccgccgagggctccatcaggtaccccgcctggaacggcatcatctcccaggaggtgctcgactacctctcctcctacatcaacaggaggatctgagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccccgggcaactttattatacaaagttgatagatatctggtctaactaactagtcctaaggacccggcggaccgattaaactgattcggtccgaagcttaagccatggcccgggaatcttagcggccgcctgcagagttaacggcgcgccgactagctagctaaggtaccgagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccac

SEQ ID NO: 13 - DNA, 19828 bp, Artificial Sequence
  Synthetic construct
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttactaccggctggatggcggggccttgatcgtgcaccgccggcgtccggactaactaactagtcgagctagttaccctatgaggtgacatgaagcgctcacggttactatgacggttagcttcacgactgttggtggcagtagcgtacgacttagctatagttccggacttacccttaagataacttcgtatagcatacattatacgaagttatgggcccaccggttacgtaacgcgtctatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcaccaggcttgggcacccgctccgggcttagaaggccagcttcaagtttgtacaaaaaagcaggcttcgaaggagatagaaccgatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgagctagacccagctttcttgtacaaagtggccgttaacggatgcagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggcaaggttgcgcccgccccagcttggtcagccgctccgggcttagaaggccgatctcgcgggcacccagctttcttctacaaagtggccgtttacggatcggctagaatgtcccgcaccggcttaccgatttgcacctcgctaccctgggttcgaccgaagctgaccgatgcttgcgcccgctcgcgaggccggccacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcccggcggatctcgactctagaggatcgctcaggaaggccgctgagatagagccatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgctatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccaacagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcactgatgtctctggaatggaaggaggaggaagtgagcatagcgttggtgcgttgctgtcaagggcgaattgtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgggtaccatttaaattgcgcccgccacggccgtggaggtcgtattccggtcagcttgcatccctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggcatggcggcgcgcagctcttcagtgtctggaacgacacttaagcgtacgtgccggcctggctctccgaaagggcgtattccagcacactggcggccgttactagacccaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggcctccggatacgtaataacttcgtatagcatacattatacgaagttatacctggagctcgtttaaacgctcttcaactggaagagcggttaccagagctggtcacctttgtccaccaagatggaactggcgcgcctcattaattaagtcagcggccgctctagttgaagacacgttcatgtcttcatcgtaagaagacactcagtagtcttcggccagaatggccatctggattcagcaggcctagaaggccatttaaatcctgaggatctggtcttcctaaggacccgggatatcgctatcaactttgtatagaaaagttgaagcttcgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctgctagcgaagttcctattccgaagttcctattctctagaaagtataggaacttcagatcctagaggatcgatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagatccatgcatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgggtaccgagctcgaattcggcccaagtttgtacaaaaaagcaggctccggccagaatggcccggaccgggttaccgaattcgcctagaaggccatttaaatcctgaggatctggtcttcctaaggacccgggatatcggaccgattaaactttaattcggtccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgacgaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggcccatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccagcttggtcacccggtccgggcctagaaggccgatctcccgggcacccagctttcttgtacaaagtggccgttaacggatcggccagaatggcccggaccgggttaccgaagttcctattccgaagttcctattctccagaaagtataggaacttcttcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatatcatgataagaaaacatactagacattaataatctgctagaaatgggaagaatcagatctaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaacaatgccccagttcgacatcctctgcaagaccccccccaaggtgctcgtgaggcagttcgtggagaggttcgagaggccctccggcgagaagatcgccctctgcgccgccgagctcacctacctctgctggatgatcacccacaacggcaccgccattaagagggccaccttcatgtcatacaacaccatcatctccaactccctctccttcgacatcgtgaacaagtccctccagttcaaatacaagacccagaaggccaccatcctcgaggcctccctcaagaagctcatccccgcctgggagttcaccatcatcccctactacggccagaagcaccagtccgacatcaccgacatcgtgtcatccctccagcttcagttcgagtcctccgaggaggctgacaagggcaactcccactccaagaagatgctgaaggccctcctctccgagggcgagtccatctgggagatcaccgagaagatcctcaactccttcgagtacacctccaggttcactaagaccaagaccctctaccagttcctcttcctcgccaccttcatcaactgcggcaggttctcagacatcaagaacgtggaccccaagtccttcaagctcgtgcagaacaagtacctaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtgtcatcatccagtgcctcgtgaccgagaccaagacctccgtgtccaggcacatctacttcttctccgctcgcggcaggatcgaccccctcgtgtacctcgacgagttcctcaggaactcagagcccgtgctcaagagggtgaacaggaccggcaactcctcctccaacaagcaggagtaccagctcctcaaggacaacctcgtgaggtcctacaacaaggccctcaagaagaacgccccctactccatcttcgccatcaagaacggccccaagtcccacatcggtaggcacctcatgacctccttcctctcaatgaagggcctcaccgagctcaccaacgtggtgggcaactggtccgacaagagggcctccgccgtggccaggaccacctacacccaccagatcaccgccatccccgaccactacttcgccctcgtgtcaaggtactacgcctacgaccccatctccaaggagatgatcgccctcaaggacgagactaaccccatcgaggagtggcagcacatcgagcagctcaagggctccgccgagggctccatcaggtaccccgcctggaacggcatcatctcccaggaggtgctcgactacctctcctcctacatcaacaggaggatctgagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgaagctgggcaactttattatacaaagttgatagatatcggaccgattaaactttaattcggtccggtggcgccgctagcgtatactgtacacctgagctgattccgatgacttcgtaggttcctagctcaagccgctcgtgtccaagcgtcacttacgattagctaatgattacggcatctaggaccgactagctaactaactagtacgtagaattaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccac

SEQ ID NO: 14 - DNA, 19416 bp, Artificial Sequence
  Synthetic construct
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttactaccggctggatggcggggccttgatcgtgcaccgccggcgtccggactaactaactagtcgagctagttaccctatgaggtgacatgaagcgctcacggttactatgacggttagcttcacgactgttggtggcagtagcgtacgacttagctatagttccggacttaccgggcccaccggtggtaccgagctcgtttaaacgctcttcaactggaagagcggttaccagagctggtcacctttgtccaccaagatggaactggcgcggctaatttaaatcctgaggatcaactttgtatagaaaagttgggccgaattcgagctcggtacggccagaatggcccggaccgggttaccgaattcgagctcggtaccctgggatccggtgcgggcctcttcgctattacgccagctggcgaaagggggatgtgctgcaaggcgattaagttgggtaacgccagggttttcccagtcacgacgttgtaaaacgacggccagtgccaagctcagatcagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaacaatgccccagttcgacatcctctgcaagaccccccccaaggtgctcgtgaggcagttcgtggagaggttcgagaggccctccggcgagaagatcgccctctgcgccgccgagctcacctacctctgctggatgatcacccacaacggcaccgccattaagagggccaccttcatgtcatacaacaccatcatctccaactccctctccttcgacatcgtgaacaagtccctccagttcaaatacaagacccagaaggccaccatcctcgaggcctccctcaagaagctcatccccgcctgggagttcaccatcatcccctactacggccagaagcaccagtccgacatcaccgacatcgtgtcatccctccagcttcagttcgagtcctccgaggaggctgacaagggcaactcccactccaagaagatgctgaaggccctcctctccgagggcgagtccatctgggagatcaccgagaagatcctcaactccttcgagtacacctccaggttcactaagaccaagaccctctaccagttcctcttcctcgccaccttcatcaactgcggcaggttctcagacatcaagaacgtggaccccaagtccttcaagctcgtgcagaacaagtacctaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtgtcatcatccagtgcctcgtgaccgagaccaagacctccgtgtccaggcacatctacttcttctccgctcgcggcaggatcgaccccctcgtgtacctcgacgagttcctcaggaactcagagcccgtgctcaagagggtgaacaggaccggcaactcctcctccaacaagcaggagtaccagctcctcaaggacaacctcgtgaggtcctacaacaaggccctcaagaagaacgccccctactccatcttcgccatcaagaacggccccaagtcccacatcggtaggcacctcatgacctccttcctctcaatgaagggcctcaccgagctcaccaacgtggtgggcaactggtccgacaagagggcctccgccgtggccaggaccacctacacccaccagatcaccgccatccccgaccactacttcgccctcgtgtcaaggtactacgcctacgaccccatctccaaggagatgatcgccctcaaggacgagactaaccccatcgaggagtggcagcacatcgagcagctcaagggctccgccgagggctccatcaggtaccccgcctggaacggcatcatctcccaggaggtgctcgactacctctcctcctacatcaacaggaggatctgagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgggtaccgagctcgaattcggcccaagtttgtacaaaaaagcaggctccggccagaatggcccggaccgggttaccgaattcgcctagaaggccatttaaatcctgaggatctggtcttcctaaggacccgggatatcggaccgattaaactttaattcggtccgaagcttcgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctgctagcgaagttcctattccgaagttcctattctctagaaagtataggaacttcagatcctagaggatcgatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagatccatgcatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccagcttggtcacccggtccgggcctagaaggccgatctcccgggcacccagctttcttgtacaaagtggccgttaacggatcggccagaatggcccggaccgggttacccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgacgaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggcccatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgaagctgggcaactttattatacaaagttgatagataaatcctgaggatctggtcttcctaaggacccgggatatcggaccgattaaactttaattcggtccgacctggtggcgccgctagcgtatacgaagttcctattccgaagttcctattctccagaaagtataggaacttcatcggaccgattaaactttaattcggtccgacctggtggcgccgctagcataacttcgtatagcatacattatacgaagttatccatatctgaccggcttaccgaatgcgtgctcgctaccctgggatgcgatttcgatggcccctggccgaagctggcatatcgctatgaccgggttagccaattcgacctcgctaccctggggatgctacgtaacgcgtctatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcaccaggcttgggcacccgctccgggcttagaaggccagcttcaagtttgtacaaaaaagcaggcttcgaaggagatagaaccgatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgagctagacccagctttcttgtacaaagtggccgttaacggatgcagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggcaaggttgcgcccgccccagcttggtcagccgctccgggcttagaaggccgatctcgcgggcacccagctttcttctacaaagtggccgtttacggatcggctagaatgtcccgcaccggcttaccgatttgcacctcgctaccctgggttcgaccgaagctgaccgatgcttgcgcccgctcgcgaggccggccacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcccggcggatctcgactctagaggatcgctcaggaaggccgctgagatagagccatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgctatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccaacagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcactgatgtctctggaatggaaggaggaggaagtgagcatagcgttggtgcgttgctgtcaagggcgaattgtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgggtaccatttaaattgcgcccgccacggccgtggaggtcgtattccggtcagcttgcatccctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggcatggcggcgcgcagctcttcagtgtctggaacgacacttaagcgtacgtgccggcctggctctccgaaagggcgtattccagcacactggcggccgttactagacccaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattagcttggtcacccggtccgggcctagaaggccagcttcggccgccccgggcaactttattatacaaagttgatagatcgaataacttcgtatagcatacattatacgaagttatcctgagctgattccgatgacttcgtaggttcctagctcaagccgctcgtgtccaagcgtcacttacgattagctaatgattacggcatctaggaccgactagctaactaactagtacgtagaattgtagaattaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccac

SEQ ID NO: 15 - DNA, 10955 bp, Artificial Sequence
  Synthetic construct
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttaccagagctggtcacctttgtccaccaagatggaactgcggccgtaagtgactagggtcacgtgaccctagtcacttatcgagctagttaccctatgaggtgacatgaagcgctcacggttactatgacggttagcttcacgactgttggtggcagtagcgtacgacttagctatagttccggacttaccgggcccaccggtggtaccgagctcgtttaaacgctcttcaactggaagagcggttaccagagctggtcacctttgtccaccaagatggaactggcgcgcctcattaattaagtcagcggccgctctagttgaagacacgttcatgtcttcatcgtaagaagacactcagtagtcttcggccagaatggccatctggattcagcaggcctagaaggccatttaaatcctgaggatctggtcttcctaaggacccgggatatcggaccgattaaactttaattcggtccgacctggtggcgccgctagcgtatacgaagttcctattccgaagttcctattctccagaaagtataggaacttctgtacaataacttcgtatagcatacattatacgaagttatatgcatacgcgtcttaagtcgatcgctatcaactttgtatagaaaagttgggccgaattcgagctcggtacggccagaatggcccggaccgggttaccgaattcgagctcggtaccctgggatccgatatcgatgggccctggccgaagcttggtcacccggtccgggcctagaaggccagcttcaagtttgtacaaaaaagcaggctccggccagaatggcccggaccggaattcgagctcggatccactagtaacggccgccagtgtgctggaattcgcccttgacggcccgggctggtatttcaaaactatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcaccaagcttggtcacccggtccgggcctagaaggccagcttcaagtttgtacaaaaaagcaggcttcgaaggagatagaaccgatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgagctagacccagctttcttgtacaaagtggccgttaacggatccagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggcaagcttgcggccgccccagcttggtcacccggtccgggcctagaaggccgatctcccgggcacccagctttcttgtacaaagtggccgttaacggatcggccagaatggcccggaccgggttaccgaattcgagctcggtaccctgggatcgaccgaagctgaccgaagcttgcggccgcacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcccgggggatctcgactctagaggatcgctcaggaaggccgctgagatagaggcatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgccatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccagcagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcactgatgtctctggaatggaaggaggaggaagtgagcatagcgttggtgcgttgctgtcaagggcgaattgtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgcgactctagatcataatcagccataccacattcgaatgtgagttgatccccggcggtgtcccccactgaagaaactatgtgctgtagtatagccgctggctagctagctagttgagtcatttagcggcgatgattgagtaataatgtgtcacgcatcaccatgcatgggtggcagtctcagtgtgagcaatgacctgaatgaacaattgaaatgaaaagaaaaaagtattgttccaaattaaacgttttaaccttttaataggtttatacaataattgatatatgttttctgtatatgtctaatttgttatcatccatttagatatagacgaaaaaaaatctaagaactaaaacaaatgctaatttgaaatgaagggagtatatattgggataatgtcgatgagatccctcgtaatatcaccgacatcacacgtgtccagttaatgtatcagtgatacgtgtattcacatttgttgcgcgtaggcgtacccaacaattttgatcgactatcagaaagtcaacggaagcgagtcgacctcgagggggggcccggtaccaagatatcaaccgcggaaagatctaagcatgcaagggcccaagtcgacctgcagaagcttcggtccgggcctagaaggccgatctcccgggcacccagctttcttgtacaaagtggccgttaacggatcggccagaatggcccggaccgggttaccgaattcgagctcggtaccctgggatcgaccgaagctgaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcctcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcggccatggcggcgcgcagctcttcagtgtctggaacgacacttaagcgtacgtgccggcctggctctccgaaagggcgaattccagcacactggcggccgttactagacccaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattagcttggtcacccggtccgggcctagaaggccagcttcggccgccccgggcaactttattatacaaagttgatagatcgaataacttcgtatagcatacattatacgaagttatcctgagctgattccgatgacttcgtaggttcctagctcaagccgctcgtgtccaagcgtcacttacgattagctaatgattacggcatctaggaccgactagctaactaactagtacaaacgtgcaagcgctactagacaattcagtacattaaaaacgtccgcaatgtgttattaagttgtctaagcgtcaatttgtttacaccacaatatatcct

SEQ ID NO: 16 - DNA, 789 bp, Escherichia coli
atgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgactgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataa

SEQ ID NO: 17 - DNA, 810 bp, Escherichia coli
atgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 18 - DNA, 14812 bp, Artificial sequence
  Synthetic construct
gctgtttaaacgctcttcaactggaagagcggttaccagaggccagaatggccatctcggaccgatatcgctatcaactttgtatagaaaagttgaagctgttacccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatcaattcgctagcgaagttcctattccgaagttcctattctctagaaagtataggaacttcagatccaccggtcgccaccatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagcggccgcaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgggtcacagcttcggccggggcccatcgatatccgcgggcatgcctgcaggtcgactctagaggatccccgggtaccgagctcgaattcggcccaagtttgtacaaaaaagcaggcttcgaaggagatagaaccagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgaccgccggggatccacacgacaccatgatagaggtgaaaccgattaacgcagaggatacctatgaactaaggcatagaatactcagaccaaaccagccgatagaagcgtgtatgtttgaaagcgatttacttcgtggtgcatttcacttaggcggctattacgggggcaaactgatttccatagcttcattccaccaggccgagcactcagaactccaaggccagaaacagtaccagctccgaggtatggctaccttggaaggttatcgtgagcagaaggcgggatcgagtctaattaaacacgctgaagaaattcttcgtaagaggggggcggacttgctttggtgtaatgcgcggacatccgcctcaggctactacaaaaagttaggcttcagcgagcagggagaggtattcgacacgccgccagtaggacctcacatcctgatgtataaaaggatcacataactagctagtcagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattcgcggccgcactcgagatatctagacccagctttcttgtacaaagtggccgttaacggatcccggtgaagttcctattccgaagttcctattctccagaaagtataggaacttcactagagcttgcggccgccccgggcaactttattatacaaagttgatagatatcggtccgagcggcctagaaggcctttggtcacctttgtccaccaagatggaactgcggccgctcattaattaagtcaggcgcgcctctagtgagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgggcccaggtagaatccgcctgagtcgcaagggtgacttcgcctatattggacgacggcgcgcagagggcgacctctttttgggttacgattgtaggattatcactaaaacaatacatgaacatattcaaatggcaatctctctaaggcattggaaataaatacaaataacagttgggtggagtttttcgacctgtgggcgttaaccttctgttaacctaaaagctcttgcccaaacagcagaatcggcgctaattgccagcggcggaacttttccagtttcgcgaaaaatatcgccactggcaaggaatgggtttgagatggcgaagtctgtcctaaaagcagcgcctgtagttgtagggttgacggccttgatggagcgtcatgccgatgccctctcgagccaacttcaagcacatcatcttaaggttttcccgccgcattccgagaagggcattcgaacattcgggccatcggaggcgtccaagctgctcggcgttggcgagtcatatttacggcagaccgcgtctgagatgccagagttgaatgttagcatgagcccgggtggcaggcgaatgttctcaattgaagatatccatgtgattcggaagtatatggatcaggtcggccgcgggaaccggcgctacctgccacatcgtcgaggcggcgagcagcttcaggttatctctgtgatgaatttcaaaggtgggtcgggtaagaccaccaccgccgcgcatctggcgcagtacctcgctatgcgcggatatcgagtcttggccattgatctcgatcctcaagcgagcctttctgcactctttgggagccaaccggagacggacgttggcccgaacgaaacgctctacggcgctataaggtatgatgatgagcaggtggcaatcgaacgagtcgtccgagggacttacattcccgacctccacctgattcctggtaaccttgagctgatggagtttgaacacgatacgccacgcgcgctgatgaaccgcaaagagggcgacacgctcttttatggtcgcatcagccaagtaattgaagatatcgcggataactatgacgtcgtggtcatcgactgccctccccagcttgggtatctcacgctatccgcattgactgcggcgacgtccattcttgtcacggtccatccgcagatgctggatgtgatgtcgatgaaccagtttctggcaatgacatcgaaccttttgcgtgaaatcgagaatgctggcgccaagttcaagtttaattggatgcgctatctgataacccgtttcgaaccgagcgacggaccacagaaccaaatggtaggttatctgcggtcgatttttggcgaaaatgtcctcaattttccgatgcttaaaaccaccgcggtttcggacgctggcctgacaaaccagactctattcgaagtggagcgtggcctgttcacgcgctcgacctatgatcgagccttggaggcgatgaacgccgtcaacgacgagatcgaaacactgatcaaaaaagcatggggtaggcccacatgagccggaagcacatccttggcgtctcaactgacgcccctgagacgtcgcccgccgacaataggacggcaaagaaccgctccatgccgctcctcggcgtaacaaggaaggagcgcgatccggcaacgaagctcacagcgaacattggtaacgcactgcgagagcaaaacgatcgtcttagccgtgccgaagagatcgagcggcgtctcgctgaaggtcaggcagtgatagagttggatgcctcgtcaatagaaccgtctttcgtgcaggatcgtatgcgaggggacattgacgggctccttacttcgatccgggaacaaggacagcaagtcccaatccttgtgcgaccgcatccgagccagccgggccgatatcaggttgccttcggccaccgccggctacgcgccgtttcagaactcggacttccggtcagagcggtcgttcgcgaactgacggacgagcaagtggtcgtagcacagggtcaggaaaacaatgagcgcgaagatcttaccttcatcgaaaaggcgcgcttcgcacatcgcctgaacaggcagttttctcgagagattgtcatcgccgcgatgtcgatcgacaagagcaatttgtccaagatgcttctgctcgttgacgccctcccctctgaactgaccgatgctattggtgccgctcctggtgttggacggccgagttggcaacaacttgccgagctgattgagaaagtttcttcaccggccgacgtggctaaatatgctatgtcggaggaagttcaagcgctgccatcggcagaacgattcaaggcggtgatcgctagtctgaagcccagtcgggttgcgcgtggacttcccgaggtcatggccaccccagacggcaccagaattgcacaggtgacgcagagcaaggccaaactggaaatcacgattgacaggaaggcgacgcccgattttgcgaccttcgtgctcgatcatgtgccagcgctgtatcaagcgtaccacgctgagaaccaacggaaacggggagagtaaaccgcaaaagaaaagagccccctcaacgtcgccgtcgcggaagcccttctgtctctctagcgcgaacagaatcgcatttcctcgaatcctcgtcaagagtttttagcgccgttttggtgagctgatttcctttgcctgctgaaaggtgaaagatgatgcagacaggaagtgtaacgacgccattcgggcggcggccaatgacgcttgcgcttgtgcggcgccagacggcgctggccgatatcaaacaaggcaagacagcggacaagtggaaggtctttagagacgcgtccgcggctatggaactacttggaatccagtccaacagtcttgccgtccttgatgcgctattgagctttcacccggaaacggagttgcgtcaggaggcacagctgatcgtcttcccgtcgaatgctcagcttgcccttcgggcgcatgggatggctggcgcgactttgcgtaggcacatcgccatgctcgtggagtcaggcttgatcgtccggaaggatagcgccaacggaaagcgttacgctcgtaaggatggcgctggtcagatcgagcgcgcgtttggcttcgatttgtctccgcttctcgcgcggtccgaagagctagcgatgatggcacagcaggtgatggccgatcgagcagcattcaggatggccaaagaaagtctgacgatttgccgacgggacgttcggaagctaattacggcagctatggaagagggagcggagggcgactggcaagctgtcgaggaagtctatgtggaacttgtgggtagaattccacgcgccccgacgcttgctgatgtagagtcaattctcgaagagatgtggatgctccaggaagagataatcaaccggttggaaattagagacaattcagaaaataatagcaccaatgctgcccagagcgagcagcacatacagaattcaaaacccgaatccgttaatgaacttgaacctcgctctgaaaaggagcagggcgctaagccgagtgaaatagaccgggcaaggagcgagccgataaaagcgttccccctcgggatgatcctgaaagcatgcccgaccattggcaactatgggccgagcggtgcggttgctagctggcgtgacctcatgtcggctgcggtggtggttcggtctatgctgggggtcagcccgtcggcttaccaagacgcgtgtgaggcaatgggaccggagaatgcggcagcagcgatggcgtgcattttggagcgagcgaacttcatcaattcgcccgggggctatctccgagatctgacacggcggagcgagcttgggaagttttcacttggcccgatgataatggcgctcttgaaggctagcgggcaggggacgttgcggtttggctagaattagcgagtatggagcaggatggtctgtggtcagctgaccacagacctaataggttgaaaacatgagcgttttttggatgatcgacagaccatccgattcccggagtaccaagcgtgctctgatgggagcgataacattactcaacaagcacgaaggccccatgccgatcgttgatcgtgaaggagagcctgctctacatgcggcggtattttgccggccgaggcatgtagtcgcggagcactgcctatttactgccctaggcacaaacgttgactcttggatcgagctggcagacaaagcaataacccacacagaggacgattaatggctgacgaagagatccagaatccgccggacggtactgctgctgccgaagttgagccggctgctcctagaggtagaagagcaaagaaagcaccagccgaaacagcccgcacgggatcgttcaaatccgtgaagccgaaaacccgcggcctcagcaaccgagaaaaactggagaagatcggtcaaatcgaagctcaggtcgctggcggcgcaaccttgaaggacgccgttaagatcgtgggtatttccgttcagacctattatcaatggaagagagctgcggttcaacctgtctcacagaatccggccgtgtctgtttcagttgacgatgaactcggcgagttcatccaactcgaggaggaaaatatgcatataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatactgcattcgctcgtgaaaagcagcgctcagggggctagcttggaagatcgcaccctgattgacatgctcgcaaccgcagttgatgtcatcgtacccttccgtgcccacggtgacatttacgaggtgggcgaaatctggctcgctgccgatgcgcgtcggcgcggtgagacaataggcgatcttcttaaccagcagtagttgtgatccatgtttctaaatgccgcatggcgcgttgtagaattacgtttgtagcaatgctcagcaatctttgtcatcaaacggagacatctagtttgcatttctgtcgtgcgcggtttggtcgaaatcttgccgaaatgcccgtgtagtgagagaaaattaaagagtggagtctagcaaatacaacctttacgtgtataaattctgttgagctgcaaatggctggccaggagcctagattgagaggtgaaccgtacgtaaccaactagtgcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagcgtaccgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttaccagagctggtcacctttgtccaccaagatggaactgcggccgtaagtgactagggtcac

SEQ ID NO: 19 - DNA, 9347 bp, Artificial Sequence
  Synthetic construct
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttactaccggttcactagctagctgctaaggttaccagagctggtcacctttgtccaccaacttattaagtatctagttgaagacacgttcttcttcacgtaagaagacactcagtagtcttcggccagaatggcctcttgattcagcgggcctagaaggccggatcactgactagctaatttaaatcctgaggatatcgctatcaactttgtatagaaaagttgaagcttcgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctgctagcgaagttcctattccgaagttcctattctctagaaagtataggaacttcagatccaccggctagaggatccaccatggttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggatccaccatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgggtaccgagctcgaattcggcccaagtttgtacaaaaaagcaggctccggccagaatggcccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaccggtcgccaccatggccctgtccaacaagttcatcggcgacgacatgaagatgacctaccacatggacggctgcgtgaacggccactacttcaccgtgaagggcgagggcagcggcaagccctacgagggcacccagacctccaccttcaaggtgaccatggccaacggcggccccctggccttctccttcgacatcctgtccaccgtgttcatgtacggcaaccgctgcttcaccgcctaccccaccagcatgcccgactacttcaagcaggccttccccgacggcatgtcctacgagagaaccttcacctacgaggacggcggcgtggccaccgccagctgggagatcagcctgaagggcaactgcttcgagcacaagtccaccttccacggcgtgaacttccccgccgacggccccgtgatggccaagaagaccaccggctgggacccctccttcgagaagatgaccgtgtgcgacggcatcttgaagggcgacgtgaccgccttcctgatgctgcagggcggcggcaactacagatgccagttccacacctcctacaagaccaagaagcccgtgaccatgccccccaaccacgtggtggagcaccgcatcgccagaaccgacctggacaagggcggcaacagcgtgcagctgaccgagcacgccgtggcccacatcacctccgtggtgcccttctgaagcggccgcaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgggcctagaaggccgatctcccgggcacccagctttcttgtacaaagtggccgttaacggatcccggtgaagttcctattccgaagttcctattctccagaaagtataggaacttcactagagcttgcggccgccccggagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaacaatgccccagttcgacatcctctgcaagaccccccccaaggtgctcgtgaggcagttcgtggagaggttcgagaggccctccggcgagaagatcgccctctgcgccgccgagctcacctacctctgctggatgatcacccacaacggcaccgccattaagagggccaccttcatgtcatacaacaccatcatctccaactccctctccttcgacatcgtgaacaagtccctccagttcaaatacaagacccagaaggccaccatcctcgaggcctccctcaagaagctcatccccgcctgggagttcaccatcatcccctactacggccagaagcaccagtccgacatcaccgacatcgtgtcatccctccagcttcagttcgagtcctccgaggaggctgacaagggcaactcccactccaagaagatgctgaaggccctcctctccgagggcgagtccatctgggagatcaccgagaagatcctcaactccttcgagtacacctccaggttcactaagaccaagaccctctaccagttcctcttcctcgccaccttcatcaactgcggcaggttctcagacatcaagaacgtggaccccaagtccttcaagctcgtgcagaacaagtacctaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtgtcatcatccagtgcctcgtgaccgagaccaagacctccgtgtccaggcacatctacttcttctccgctcgcggcaggatcgaccccctcgtgtacctcgacgagttcctcaggaactcagagcccgtgctcaagagggtgaacaggaccggcaactcctcctccaacaagcaggagtaccagctcctcaaggacaacctcgtgaggtcctacaacaaggccctcaagaagaacgccccctactccatcttcgccatcaagaacggccccaagtcccacatcggtaggcacctcatgacctccttcctctcaatgaagggcctcaccgagctcaccaacgtggtgggcaactggtccgacaagagggcctccgccgtggccaggaccacctacacccaccagatcaccgccatccccgaccactacttcgccctcgtgtcaaggtactacgcctacgaccccatctccaaggagatgatcgccctcaaggacgagactaaccccatcgaggagtggcagcacatcgagcagctcaagggctccgccgagggctccatcaggtaccccgcctggaacggcatcatctcccaggaggtgctcgactacctctcctcctacatcaacaggaggatctgagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccccgggcaactttattatacaaagttgatagatatctggtctaactaactagtcctaaggacccggcggaccgattaaactgattcggtccgaagcttaagccatggcccgggaatcttagcggccgcctgcagagttaacggcgcgccgactagctagctaaggtaccgagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccac

SEQ ID NO: 20 - DNA, 16939 bp, Artificial Sequence
  Synthetic construct
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttaccagagctggtcacctttgtccaccaagatggaactgcggccgctcattaattaagtcaggcgcgcctctagttgaagacacgttcatgtcttcatcgtaagaagacactcagtagtcttcggccagaatggcccggaccgggttacccggtccggaattcgagctccaccgcggtggcggccgctctagattatataatttataagctaaacaacccggccctaaagcactatcgtatcacctatctaaataagtcacgggagtttcgaacgtccacttcgtcgcacggaattgcatgtttcttgttggaagcatattcacgcaatctccacacataaaggtttatgtataaacttacatttagctcagtttaattacagtcttatttggatgcatatgtatggttctcaatccatataagttagagtaaaaaataagtttaaattttatcttaattcactccaacatatatggatctacaatactcatgtgcatccaaacaaactacttatattgaggtgaatttggtagaaattaaactaacttacacactaagccaatctttactatattaaagcaccagtttcaacgatcgtcccgcgtcaatattattaaaaaactcctacatttctttataatcaacccgcactcttataatctcttctctactactataataagagagtttatgtacaaaataaggtgaaattatctataagtgttctggatattggttgttggctcccatattcacacaacctaatcaatagaaaacatatgttttattaaaacaaaatttatcatatatcatatatatatatatatcatatatatatataaaccgtagcaatgcacgggcatataactagtgcaacttaatacatgtgtgtattaagatgaataagagggtatccaaataaaaaacttgttgcttacgtatggatcgaaaggggttggaaacgattaaacgattaaatctcttcctagtcaaaattgaatagaaggagatttaatatatcccaatccccttcgatcatccaggtgcaaccgtataagtcctaaagtggtgaggaacacgaaagaaccatgcattggcatgtaaagctccaagaatttgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtatcggtggcataagaacacaagaaattgtgttaattaatcaaagctataaataacgctcgcatgcctgtgcacttctccatcaccaccactgggtcttcagaccattagctttatctactccagagcgcagaagaacccgatcgacagatatcggatccaccggtcgccaccatggccctgtccaacaagttcatcggcgacgacatgaagatgacctaccacatggacggctgcgtgaacggccactacttcaccgtgaagggcgagggcagcggcaagccctacgagggcacccagacctccaccttcaaggtgaccatggccaacggcggccccctggccttctccttcgacatcctgtccaccgtgttcatgtacggcaaccgctgcttcaccgcctaccccaccagcatgcccgactacttcaagcaggccttccccgacggcatgtcctacgagagaaccttcacctacgaggacggcggcgtggccaccgccagctgggagatcagcctgaagggcaactgcttcgagcacaagtccaccttccacggcgtgaacttccccgccgacggccccgtgatggccaagaagaccaccggctgggacccctccttcgagaagatgaccgtgtgcgacggcatcttgaagggcgacgtgaccgccttcctgatgctgcagggcggcggcaactacagatgccagttccacacctcctacaagaccaagaagcccgtgaccatgccccccaaccacgtggtggagcaccgcatcgccagaaccgacctggacaagggcggcaacagcgtgcagctgaccgagcacgccgtggcccacatcacctccgtggtgcccttctgaagcggccaacgatccccggcggtgtcccccactgaagaaactatgtgctgtagtatagccgctgcccgctggctagctagctagttgagtcatttagcggcgatgattgagtaataatgtgtcacgcatcaccatgcatgggtggcagtctcagtgtgagcaatgacctgaatgaacaattgaaatgaaaagaaaaaagtattgttccaaattaaacgttttaaccttttaataggtttatacaataattgatatatgttttctgtatatgtctaatttgttatcatccatttagatatagacgaaaaaaaatctaagaactaaaacaaatgctaatttgaaatgaagggagtatatattgggataatgtcgatgagatccctcgtaatatcaccgacatcacacgtgtccagttaatgtatcagtgatacgtgtattcacatttgttgcgcgtaggcgtacccaacaattttgatcgactatcagaaagtcaacggaagcgagtcgacctcgagggggggccccggccgaagcttcggaccgggtcacccggtccgggcctagaaggccatttaaatcctgaggatctggtcttcctaaggacccgggatatcgctatcaactttgtatagaaaagttgggccgaattcgagctcggtacggccagaatggcccggaccgaagcttcggccgcacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattactctatcagtgatagagtgggggatctcgactctagaggatcgctcaggaaggccgctgagatagaggcatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgccatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccagcagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcactgatgtctctggaatggaaggaggaggaagtgagcatagcgttggtgcgttgctgtcaagggcgaattcacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgccaccgcggtggagctcgaattccggtccgggcctagaaggccagcttcaagtttgtacaaaaaagcaggctccggccagaatggcccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccactctatcagtgatagagtgtaataaatagactctatcagtgatagagtgaactctatcagtgatagagtacaccccctccacaccctctttccccaacctcgtgttgttcggagcgcacacacacacaaccagatctcccccaaatccacccgtcggcacctccgcttcaagaggtacggcgatcgatcatcccccctccttctctctaccttcttttctctagactacatcggatggcgatccatggttagggcctgctagtttcccttcctgttttgtcgatggctgcgaggcacaatagatctgatggcgttatgacggctaacttgtcatgttgttgcgatttatagtccctttaggagatcagtttaatttctcggatggttcgagatcggtggtccatggttagtaccctaagatccgcgctgttagggttcgtagatggaggcgacctgttctgattgttaacttgtcagtacctgggaaatcctgggatggttctagctcgtccgcagatgagatcgatttcatgatcctctgtatcttgtttcgttgcctaggttccgtctaatctatccgtggtatgatgtagatgttttgatcgtgctaactacgtcttgtaaagttaattgtcaggtcataatttttagcatgcctttttttttgtttggttttgtctaattgggctgtcgttctagatcagagtagaagactgttccaaactacctgctggatttattgaacttggatctgtatgtgtgtcacatatcttcataaattcatgattaagatggattgaaatatcttttatctttttggtatggatagttctatatgttggtgtggctttgttagatgtatacatgcttagatacatgaagcaacgtgctgctactgtttagtaattgctgttcatttgtctaataaacagataaggatatgtatttatgttgctgttggttttgctggtactttgttggatacaaatgcttcaatacagaaaacagcatgctgctacgatttaccatttatctaatcttatcatatgtctaatctaataaacaaacatgcttttaaattatcttcatatgcttggatgatggcatacacagcggctatgtgtggttttttaaatacccagcatcatgggcatgcatgacactgctttaatatgctttttatttgcttgagactgtttcttttgtttatactgaccctttagttcggtgactcttctgcagatccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcctcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcggccatggcggcgcgcagctcttcagtgtctggaacgacacttaagcgtacgtgccggcctggctctccgaaagggcgaattccagcacactggcggccgttactagacccaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgggcctagaaggccgatctcccgggcacccagctttcttgtacaaagtggccgttaacggatcggccagaatggcccggaccgggttaccgaattcgagctcggtaccctgggatcggccgctctagaactagtggatcccggccgtgatccccggcggtgtcccccactgaagaaactatgtgctgtagtatagccgctgcccgctggctagctagctagttgagtcatttagcggcgatgattgagtaataatgtgtcacgcatcaccatgcatgggtggcagtctcagtgtgagcaatgacctgaatgaacaattgaaatgaaaagaaaaaagtattgttccaaattaaacgttttaaccttttaataggtttatacaataattgatatatgttttctgtatatgtctaatttgttatcatccatttagatatagacgaaaaaaaatctaagaactaaaacaaatgctaatttgaaatgaagggagtatatattgggataatgtcgatgagatccctcgtaatatcaccgacatcacacgtgtccagttaatgtatcagtgatacgtgtattcacatttgttgcgcgtaggcgtacccaacaattttgatcgactatcagaaagtccggtccgctcgaggcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatggccagactcgacaagagcaaggtgatcaacagcgcactggagctgctgaacgaggtcggaatcgaaggcctcacaacccgtaagctcgcccagaagctcggggttgagcagcctacattgtactggcacgtcaagaacaagcgagctctgctagacgctatggccatagagatgctcgatccgcacaagattcactacttacccttggaaggggaaagctggcaagatttcttgaggaacagggctaagtccatgagaaatgctttgctcagtcaccgtgatggagccaaggtctgtctaggtacgggcttcacggagcgacaatatgaaactgctgagaacacgcttgccttcctgacacaacaaggtttctcccttgagaacgccctctacgcattccaagcagtggggatctacactctgggttgtgtcttgctggatcaagagctgcaagtcgctaaggaggagagggaaacacctactactgatagtatgccgccactggttcgacaagcttacgaactcgcggatcaccaaggtgcagagccagccttcctgttcggccttgaactgatcatatcaggattggagaagcagctgaaggccgaaagtgggtcttaatgatagctgcagaaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattcctgcagcccaacggatcggccagaatggcccggaccgggttaccgaattcgagctcggtaccctgggatctgcccttactttaacgcctctaaccaacacccctttatctttataaggaacaataaacagaatttgccccactgttctaaatcacctaataatatccccagctaaaaacaataaaggtttcctagaattaagacaagcatgactgttcctccaggagggtttggaacattgttgcagtcttgcagatacgggcgaagggtgagaaacagagcggagggctggaggtgacctcggtagtcgacgccggagttgagcttgacaacgacggggcggcccctgatggacttgaggaagtccgatggcgtcttcaccgtcccgccggcgcccgaggcgggcctgtcgctgccgccgccgccgctgctcatcttgcgcgctgtgcccccggcggtgtccctgtgttgcggatcgcgggtgggccaggtggatgcgagggcgacccgtttggactccggccggagccgccggatccctggtcggtgtcagtgccgtttactctgggccccacgtgtcagtaccgtctgtagatgacaacaacccgtcgtccacagtcatgtccaaaatatcctttcttcttttttttcgattcggatatctatcttccttttttttttccaaaaatcttcttgacgcaccagcgcgcacgtttgtggtaaacgccgacacgtcggtcccacgtcgatagaccccacccaccagtgagtagcgtgtacgtattcgggggtgacggacgtgtcgccgtcgtcttgctagtcccattcccatctgagccacacatctctgaacaaaaaaaaggagggaggcctccacgcacatccccctccgtgccacccgccccaaaccctcgcgccgcctccgagacagccgccgcaaccatggccaccgccgccgccgcgtctaccgcgctcactggcgccactaccgctgcgcccaaggcgaggcgccgggcgcacctcctggccacccgccgcgccctcgccgcgcccatcaggtgctcagcggcgtcacccgccatgccgatggctcccccggccaccccgctccggccgtggggccccaccgagccccgcaagggtgctgacatcctcgtcgagtccctcgagcgctgcggcgtccgcgacgtcttcgcctaccccggcggcgcgtccatggagatccaccaggcactcacccgctcccccgtcatcgccaaccacctcttccgccacgagcaaggggaggcctttgccgcctccggctacgcgcgctcctcgggccgcgtcggcgtctgcatcgccacctccggccccggcgccaccaacctagtctccgcgctcgccgacgcgctgctcgattccgtccccatggtcgccatcacgggacaggtggcgcgacgcatgattggcaccgacgccttccaggagacgcccatcgtcgaggtcacccgctccatcaccaagcacaactacctggtcctcgacgtcgacgacatcccccgcgtcgtgcaggaggctttcttcctcgcctcctctggtcgaccagggccggtgcttgtcgacatccccaaggacatccagcagcagatggcggtgcctgtctgggacaagcccatgagtctgcctgggtacattgcgcgccttcccaagccccctgcgactgagttgcttgagcaggtgctgcgtcttgttggtgaatcgcggcgccctgttctttatgtgggcggtggctgcgcagcatctggtgaggagttgcgacgctttgtggagctgactggaatcccggtcacaactactcttatgggcctcggcaacttccccagcgacgacccactgtctctgcgcatgctaggtatgcatgggacggtgtatgcaaattatgcagtggataaggccgatctgttgcttgcacttggtgtgcggtttgatgatcgcgtgacagggaagattgaggcttttgcaagcagggctaagattgtgcacgttgatattgatccggctgagattggcaagaacaagcagccacatgtgtccatctgtgcagatgttaagcttgctttgcagggcatgaatgctcttcttgaaggaagcacatcaaagaagagctttgactttggctcatggaacgatgagttggatcagcagaagagggaattcccccttgggtataaaacatctaatgaggagatccagccacaatatgctattcaggttcttgatgagctgacgaaaggcgaggccatcatcggcacaggtgttgggcagcaccagatgtgggcggcacagtactacacttacaagcggccaaggcagtggttgtcttcagctggtcttggggctatgggatttggtttgccggctgctgctggtgcttctgtggcaaacccaggtgtcactgttgttgacatcgatggagatggtagctttctcatgaacgttcaggagctagctatgatccgaattgagaacctcccagtgaaggtctttgtgctaaacaaccagcacctggggatggtggtgcagttggaggacaggttctataaggccaacagagcgcacacatacttgggaaacccagagaatgaaagtgagatatatccagatttcgtgacgatcgccaaagggttcaacattccagcggtccgtgtgacaaagaagaacgaagtccgcgcagcgataaagaagatgctcgagactccagggccgtacctcttggatataatcgtcccacaccaggagcatgtgttgcctatgatccctagtggtggggctttcaaggatatgatcctggatggtgatggcaggactgtgtactgactagctagtcagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattcctgcagcccgggcaactttattatacaaagttgatagatctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccac

SEQ ID NO: 21 - DNA, 20480 bp, Artificial Sequence
  Synthetic construct
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttactaccggctggatggcggggccttgatcgtgcaccgccggcgtccggactaactaactagtcgagctagttaccctatgaggtgacatgaagcgctcacggttactatgacggttagcttcacgactgttggtggcagtagcgtacgacttagctatagttccggacttaccgggcccaccggtggtaccgagctcgtttaaacgctcttcaactggaagagcggttaccagagctggtcacctttgtccaccaagatggaactggcgcggctaatttaaatcctgaggatcaactttgtatagaaaagttgggccgaattcgagctcggtacggccagaatggcccggaccgggttaccgaattcgagctcggtaccctgggatccggtgcgggcctcttcgctattacgccagctggcgaaagggggatgtgctgcaaggcgattaagttgggtaacgccagggttttcccagtcacgacgttgtaaaacgacggccagtgccaagctcagatcagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaacaatgccccagttcgacatcctctgcaagaccccccccaaggtgctcgtgaggcagttcgtggagaggttcgagaggccctccggcgagaagatcgccctctgcgccgccgagctcacctacctctgctggatgatcacccacaacggcaccgccattaagagggccaccttcatgtcatacaacaccatcatctccaactccctctccttcgacatcgtgaacaagtccctccagttcaaatacaagacccagaaggccaccatcctcgaggcctccctcaagaagctcatccccgcctgggagttcaccatcatcccctactacggccagaagcaccagtccgacatcaccgacatcgtgtcatccctccagcttcagttcgagtcctccgaggaggctgacaagggcaactcccactccaagaagatgctgaaggccctcctctccgagggcgagtccatctgggagatcaccgagaagatcctcaactccttcgagtacacctccaggttcactaagaccaagaccctctaccagttcctcttcctcgccaccttcatcaactgcggcaggttctcagacatcaagaacgtggaccccaagtccttcaagctcgtgcagaacaagtacctaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtgtcatcatccagtgcctcgtgaccgagaccaagacctccgtgtccaggcacatctacttcttctccgctcgcggcaggatcgaccccctcgtgtacctcgacgagttcctcaggaactcagagcccgtgctcaagagggtgaacaggaccggcaactcctcctccaacaagcaggagtaccagctcctcaaggacaacctcgtgaggtcctacaacaaggccctcaagaagaacgccccctactccatcttcgccatcaagaacggccccaagtcccacatcggtaggcacctcatgacctccttcctctcaatgaagggcctcaccgagctcaccaacgtggtgggcaactggtccgacaagagggcctccgccgtggccaggaccacctacacccaccagatcaccgccatccccgaccactacttcgccctcgtgtcaaggtactacgcctacgaccccatctccaaggagatgatcgccctcaaggacgagactaaccccatcgaggagtggcagcacatcgagcagctcaagggctccgccgagggctccatcaggtaccccgcctggaacggcatcatctcccaggaggtgctcgactacctctcctcctacatcaacaggaggatctgagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgggtaccgagctcgaattcggcccaagtttgtacaaaaaagcaggctccggccagaatggcccggaccgggttaccgaattcgcctagaaggccatttaaatcctgaggatctggtcttcctaaggacccgggatatcggaccgattaaactttaattcggtccgaagcttcgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctgctagcgaagttcctattccgaagttcctattctctagaaagtataggaacttcagatcctagaggatcgatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaaatccatcatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccagcttggtcacccggtccgggcctagaaggccgatctcccgggcacccagctttcttgtacaaagtggccgttaacggatcggccagaatggcccggaccgggttacccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgacgaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggcccatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgaagctgggcaactttattatacaaagttgatagataaatcctgaggatctggtcttcctaaggacccgggatatcggaccgattaaactttaattcggtccgacctggtggcgccgctagcgtatacgaagttcctattccgaagttcctattctccagaaagtataggaacttcatcggaccgattaaactttaattcggtccgacctggtggcgccgctagcataacttcgtatagcatacattatacgaagttatccatatctgaccggcttaccgaatgcgtgctcgctaccctgggatgcgatttcgatggcccctggccgaagctggcatatcgctatgaccgggttagccaattcgacctcgctaccctggggatgctacgtaacgcgtctatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcaccaggcttgggcacccgctccgggcttagaaggccagcttcaagtttgtacaaaaaagcaggcttcgaaggagatagaaccgatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgagctagacccagctttcttgtacaaagtggccgttaacggatgcagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggcaaggttgcgcccgccccagcttggtcagccgctccgggcttagaaggccgatctcgcgggcacccagctttcttctacaaagtggccgtttacggatcggctagaatgtcccgcaccggcttaccgatttgcacctcgctaccctgggttcgaccgaagctgaccgatgcttgcgcccgctcgcgaggccggccaagcttgatatcgaattcctgcagcccatccctcagccgcctttcactatcttttttgcccgagtcattgtcatgtgaaccttggcatgtataatcggtgaattgcgtcgattttcctcttataggtgggccaatgaatccgtgtgatcgcgtctgattggctagagatatgtttcttccttgttggatgtattttcatacataatcatatgcatacaaatatttcattacactttatagaaatggtcagtaataaaccctatcactatgtctggtgtttcattttatttgcttttaaacgaaaattgacttcctgattcaatatttaaggatcgtcaacggtgtgcagttactaaattctggtttgtaggaactatagtaaactattcaagtcttcacttattgtgcactcacctctcgccacatcaccacagatgttattcacgtcttaaatttgaactacacatcatattgacacaatattttttttaaataagcgattaaaacctagcctctatgtcaacaatggtgtacataaccagcgaagtttagggagtaaaaaacatcgccttacacaaagttcgctttaaaaaataaagagtaaattttactttggaccacccttcaaccaatgtttcactttagaacgagtaattttattattgtcactttggaccaccctcaaatcttttttccatctacatccaatttatcatgtcaaagaaatggtctacatacagctaaggagatttatcgacgaatagtagctagcatactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtcacggtctcgatctttggccttggtagtttgggtgggcgagaggcggcttcgtgcgcgcccagatcggtgcgcgggaggggcgggatctcgcggctggggctctcgccggcgtggatcaggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaaggatccatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgctatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccaacagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcggcgatcgcggtaccatttaaattgcgcccgccacggccgtggaggtcgtattccggtcagcttgcatccctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggcatggcggcgcgcagctcttcagtgtctggaacgacacttaagcgtacgtgccggcctggctctccgaaagggcgtattccagcacactggcggccgttactagacccaacctagacttgtccatcttctggattggccaacttaattaatgtatgaaataaaaggatgcacacatagtgacatgctaatcact

SEQ ID NO: 22 - DNA, 19477 bp, Artificial sequence
  Synthetic construct
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttactaccggctggatggcggggccttgatcgtgcaccgccggcgtccggactaactaactagtcgagctagttaccctatgaggtgacatgaagcgctcacggttactatgacggttagcttcacgactgttggtggcagtagcgtacgacttagctatagttccggacttaccgggcccaccggtggtaccgagctcgtttaaacgctcttcaactggaagagcggttaccagagctggtcacctttgtccaccaagatggaactggcgcggctaatttaaatcctgaggatcaactttgtatagaaaagttgggccgaattcgagctcggtacggccagaatggcccggaccgggttaccgaattcgagctcggtaccctgggatccggtgcgggcctcttcgctattacgccagctggcgaaagggggatgtgctgcaaggcgattaagttgggtaacgccagggttttcccagtcacgacgttgtaaaacgacggccagtgccaagctcagatcagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaacaatgccccagttcgacatcctctgcaagaccccccccaaggtgctcgtgaggcagttcgtggagaggttcgagaggccctccggcgagaagatcgccctctgcgccgccgagctcacctacctctgctggatgatcacccacaacggcaccgccattaagagggccaccttcatgtcatacaacaccatcatctccaactccctctccttcgacatcgtgaacaagtccctccagttcaaatacaagacccagaaggccaccatcctcgaggcctccctcaagaagctcatccccgcctgggagttcaccatcatcccctactacggccagaagcaccagtccgacatcaccgacatcgtgtcatccctccagcttcagttcgagtcctccgaggaggctgacaagggcaactcccactccaagaagatgctgaaggccctcctctccgagggcgagtccatctgggagatcaccgagaagatcctcaactccttcgagtacacctccaggttcactaagaccaagaccctctaccagttcctcttcctcgccaccttcatcaactgcggcaggttctcagacatcaagaacgtggaccccaagtccttcaagctcgtgcagaacaagtacctaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtgtcatcatccagtgcctcgtgaccgagaccaagacctccgtgtccaggcacatctacttcttctccgctcgcggcaggatcgaccccctcgtgtacctcgacgagttcctcaggaactcagagcccgtgctcaagagggtgaacaggaccggcaactcctcctccaacaagcaggagtaccagctcctcaaggacaacctcgtgaggtcctacaacaaggccctcaagaagaacgccccctactccatcttcgccatcaagaacggccccaagtcccacatcggtaggcacctcatgacctccttcctctcaatgaagggcctcaccgagctcaccaacgtggtgggcaactggtccgacaagagggcctccgccgtggccaggaccacctacacccaccagatcaccgccatccccgaccactacttcgccctcgtgtcaaggtactacgcctacgaccccatctccaaggagatgatcgccctcaaggacgagactaaccccatcgaggagtggcagcacatcgagcagctcaagggctccgccgagggctccatcaggtaccccgcctggaacggcatcatctcccaggaggtgctcgactacctctcctcctacatcaacaggaggatctgagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgggtaccgagctcgaattcggcccaagtttgtacaaaaaagcaggctccggccagaatggcccggaccgggttaccgaattcgcctagaaggccatttaaatcctgaggatctggtcttcctaaggacccgggatatcggaccgattaaactttaattcggtccgaagcttcgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctgctagcgaagttcctattccgaagttcctattctctagaaagtataggaacttcagatcctagaggatcgatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaaatccatcatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccagcttggtcacccggtccgggcctagaaggccgatctcccgggcacccagctttcttgtacaaagtggccgttaacggatcggccagaatggcccggaccgggttacccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgacgaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggcccatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgaagctgggcaactttattatacaaagttgatagataaatcctgaggatctggtcttcctaaggacccgggatatcggaccgattaaactttaattcggtccgacctggtggcgccgctagcgtatacgaagttcctattccgaagttcctattctccagaaagtataggaacttcatcggaccgattaaactttaattcggtccgacctggtggcgccgctagcataacttcgtatagcatacattatacgaagttatccatatctgaccggcttaccgaatgcgtgctcgctaccctgggatgcgatttcgatggcccctggccgaagctggcatatcgctatgaccgggttagccaattcgacctcgctaccctggggatgctacgtaacgcgtctatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcaccaggcttgggcacccgctccgggcttagaaggccagcttcaagtttgtacaaaaaagcaggcttcgaaggagatagaaccgatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgagctagacccagctttcttgtacaaagtggccgttaacggatgcagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggcaaggttgcgcccgccccagcttggtcagccgctccgggcttagaaggccgatctcgcgggcacccagctttcttctacaaagtggccgtttacggatcggctagaatgtcccgcaccggcttaccgatttgcacctcgctaccctgggttcgaccgaagctgaccgatgcttgcgcccgctcgcgaggccggccaagcttccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacaggatccatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgctatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccaacagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcggcgatcgcggtaccatttaaattgcgcccgccacggccgtggaggtcgtattccggtcagcttgcatccctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggcatggcggcgcgcagctcttcagtgtctggaacgacacttaagcgtacgtgccggcctggctctccgaaagggcgtattccagcacactggcggccgttactagacccaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattagcttggtcacccggtccgggcctagaaggccagcttcggccgccccgggcaactttattatacaaagttgatagatcgaataacttcgtatagcatacattatacgaagttatcctgagctgattccgatgacttcgtaggttcctagctcaagccgctcgtgtccaagcgtcacttacgattagctaatgattacggcatctaggaccgactagctaactaactagtacgtagaattgtagaattaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccac

SEQ ID NO: 23 - DNA, 21610 bp, Artificial sequence
  Synthetic construct
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttactaccggctggatggcggggccttgatcgtgcaccgccggcgtccggactaactaactagtcgagctagttaccctatgaggtgacatgaagcgctcacggttactatgacggttagcttcacgactgttggtggcagtagcgtacgacttagctatagttccggacttaccgggcccaccggtggtaccgagctcgtttaaacgctcttcaactggaagagcggttaccagagctggtcacctttgtccaccaagatggaactggcgcggctaatttaaatcctgaggatcaactttgtatagaaaagttgggccgaattcgagctcggtacggccagaatggcccggaccgggttaccgaattcgagctcggtaccctgggatccggtgcgggcctcttcgctattacgccagctggcgaaagggggatgtgctgcaaggcgattaagttgggtaacgccagggttttcccagtcacgacgttgtaaaacgacggccagtgccaagctcagatcagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaacaatgccccagttcgacatcctctgcaagaccccccccaaggtgctcgtgaggcagttcgtggagaggttcgagaggccctccggcgagaagatcgccctctgcgccgccgagctcacctacctctgctggatgatcacccacaacggcaccgccattaagagggccaccttcatgtcatacaacaccatcatctccaactccctctccttcgacatcgtgaacaagtccctccagttcaaatacaagacccagaaggccaccatcctcgaggcctccctcaagaagctcatccccgcctgggagttcaccatcatcccctactacggccagaagcaccagtccgacatcaccgacatcgtgtcatccctccagcttcagttcgagtcctccgaggaggctgacaagggcaactcccactccaagaagatgctgaaggccctcctctccgagggcgagtccatctgggagatcaccgagaagatcctcaactccttcgagtacacctccaggttcactaagaccaagaccctctaccagttcctcttcctcgccaccttcatcaactgcggcaggttctcagacatcaagaacgtggaccccaagtccttcaagctcgtgcagaacaagtacctaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtgtcatcatccagtgcctcgtgaccgagaccaagacctccgtgtccaggcacatctacttcttctccgctcgcggcaggatcgaccccctcgtgtacctcgacgagttcctcaggaactcagagcccgtgctcaagagggtgaacaggaccggcaactcctcctccaacaagcaggagtaccagctcctcaaggacaacctcgtgaggtcctacaacaaggccctcaagaagaacgccccctactccatcttcgccatcaagaacggccccaagtcccacatcggtaggcacctcatgacctccttcctctcaatgaagggcctcaccgagctcaccaacgtggtgggcaactggtccgacaagagggcctccgccgtggccaggaccacctacacccaccagatcaccgccatccccgaccactacttcgccctcgtgtcaaggtactacgcctacgaccccatctccaaggagatgatcgccctcaaggacgagactaaccccatcgaggagtggcagcacatcgagcagctcaagggctccgccgagggctccatcaggtaccccgcctggaacggcatcatctcccaggaggtgctcgactacctctcctcctacatcaacaggaggatctgagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgggtaccgagctcgaattcggcccaagtttgtacaaaaaagcaggctccggccagaatggcccggaccgggttaccgaattcgcctagaaggccatttaaatcctgaggatctggtcttcctaaggacccgggatatcggaccgattaaactttaattcggtccgaagcttcgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctgctagcgaagttcctattccgaagttcctattctctagaaagtataggaacttcagatcctagaggatcgatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagatccatgcatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccagcttggtcacccggtccgggcctagaaggccgatctcccgggcacccagctttcttgtacaaagtggccgttaacggatcggccagaatggcccggaccgggttacccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgacgaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggcccatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgaagctgggcaactttattatacaaagttgatagataaatcctgaggatctggtcttcctaaggacccgggatatcggaccgattaaactttaattcggtccgacctggtggcgccgctagcgtatacgaagttcctattccgaagttcctattctccagaaagtataggaacttcatcggaccgattaaactttaattcggtccgacctggtggcgccgctagcataacttcgtatagcatacattatacgaagttatccatatctgaccggcttaccgaatgcgtgctcgctaccctgggatgcgatttcgatggcccctggccgaagctggcatatcgctatgaccgggttagccaattcgacctcgctaccctggggatgctacgtaacgcgtctatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcaccaggcttgggcacccgctccgggcttagaaggccagcttcaagtttgtacaaaaaagcaggcttcgaaggagatagaaccgatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgagctagacccagctttcttgtacaaagtggccgttaacggatgcagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggcaaggttgcgcccgccccagcttggtcagccgctccgggcttagaaggccgatctcgcgggcacccagctttcttctacaaagtggccgtttacggatcggctagaatgtcccgcaccggcttaccgatttgcacctcgctaccctgggttcgaccgaagctgaccgatgcttgcgcccgctcgcgaggccggccaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgctatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccaacagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcggcgatcgcggtaccatttaaattgcgcccgccacggccgtggaggtcgtattccggtcagcttgcatccctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggcatggcggcgcgcagctcttcagtgtctggaacgacacttaagcgtacgtgccggcctggctctccgaaagggcgtattccagcacactggcggccgttactagacccaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattagcttggtcacccggtccgggcctagaaggccagcttcggccgccccgggcaactttattatacaaagttgatagatcgaataacttcgtatagcatacattatacgaagttatcctgagctgattccgatgacttcgtaggttcctagctcaagccgctcgtgtccaagcgtcacttacgattagctaatgattacggcatctaggaccgactagctaactaactagtacgtagaattgtagaattaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccacataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattagcttggtcacccggtccgggcctagaaggccagcttcggccgccccgggcaactttattatacaaagttgatagatcgaataacttcgtatagcatacattatacgaagttatcctgagctgattccgatgacttcgtaggttcctagctcaagccgctcgtgtccaagcgtcacttacgattagctaatgattacggcatctaggaccgactagctaactaactagtacgtagaattgtagaattaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccac

SEQ ID NO: 24 - DNA, 19866 bp, Artificial sequence
  Synthetic construct
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttactaccggctggatggcggggccttgatcgtgcaccgccggcgtccggactaactaactagtcgagctagttaccctatgaggtgacatgaagcgctcacggttactatgacggttagcttcacgactgttggtggcagtagcgtacgacttagctatagttccggacttaccgggcccaccggtggtaccgagctcgtttaaacgctcttcaactggaagagcggttaccagagctggtcacctttgtccaccaagatggaactggcgcggctaatttaaatcctgaggatcaactttgtatagaaaagttgggccgaattcgagctcggtacggccagaatggcccggaccgggttaccgaattcgagctcggtaccctgggatccggtgcgggcctcttcgctattacgccagctggcgaaagggggatgtgctgcaaggcgattaagttgggtaacgccagggttttcccagtcacgacgttgtaaaacgacggccagtgccaagctcagatcagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaacaatgccccagttcgacatcctctgcaagaccccccccaaggtgctcgtgaggcagttcgtggagaggttcgagaggccctccggcgagaagatcgccctctgcgccgccgagctcacctacctctgctggatgatcacccacaacggcaccgccattaagagggccaccttcatgtcatacaacaccatcatctccaactccctctccttcgacatcgtgaacaagtccctccagttcaaatacaagacccagaaggccaccatcctcgaggcctccctcaagaagctcatccccgcctgggagttcaccatcatcccctactacggccagaagcaccagtccgacatcaccgacatcgtgtcatccctccagcttcagttcgagtcctccgaggaggctgacaagggcaactcccactccaagaagatgctgaaggccctcctctccgagggcgagtccatctgggagatcaccgagaagatcctcaactccttcgagtacacctccaggttcactaagaccaagaccctctaccagttcctcttcctcgccaccttcatcaactgcggcaggttctcagacatcaagaacgtggaccccaagtccttcaagctcgtgcagaacaagtacctaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtgtcatcatccagtgcctcgtgaccgagaccaagacctccgtgtccaggcacatctacttcttctccgctcgcggcaggatcgaccccctcgtgtacctcgacgagttcctcaggaactcagagcccgtgctcaagagggtgaacaggaccggcaactcctcctccaacaagcaggagtaccagctcctcaaggacaacctcgtgaggtcctacaacaaggccctcaagaagaacgccccctactccatcttcgccatcaagaacggccccaagtcccacatcggtaggcacctcatgacctccttcctctcaatgaagggcctcaccgagctcaccaacgtggtgggcaactggtccgacaagagggcctccgccgtggccaggaccacctacacccaccagatcaccgccatccccgaccactacttcgccctcgtgtcaaggtactacgcctacgaccccatctccaaggagatgatcgccctcaaggacgagactaaccccatcgaggagtggcagcacatcgagcagctcaagggctccgccgagggctccatcaggtaccccgcctggaacggcatcatctcccaggaggtgctcgactacctctcctcctacatcaacaggaggatctgagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgggtaccgagctcgaattcggcccaagtttgtacaaaaaagcaggctccggccagaatggcccggaccgggttaccgaattcgcctagaaggccatttaaatcctgaggatctggtcttcctaaggacccgggatatcggaccgattaaactttaattcggtccgaagcttcgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctgctagcgaagttcctattccgaagttcctattctctagaaagtataggaacttcagatcctagaggatcgatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagatccatgcatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccagcttggtcacccggtccgggcctagaaggccgatctcccgggcacccagctttcttgtacaaagtggccgttaacggatcggccagaatggcccggaccgggttacccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgacgaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggcccatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgaagctgggcaactttattatacaaagttgatagataaatcctgaggatctggtcttcctaaggacccgggatatcggaccgattaaactttaattcggtccgacctggtggcgccgctagcgtatacgaagttcctattccgaagttcctattctccagaaagtataggaacttcatcggaccgattaaactttaattcggtccgacctggtggcgccgctagcataacttcgtatagcatacattatacgaagttatccatatctgaccggcttaccgaatgcgtgctcgctaccctgggatgcgatttcgatggcccctggccgaagctggcatatcgctatgaccgggttagccaattcgacctcgctaccctggggatgctacgtaacgcgtctatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcaccaggcttgggcacccgctccgggcttagaaggccagcttcaagtttgtacaaaaaagcaggcttcgaaggagatagaaccgatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgagctagacccagctttcttgtacaaagtggccgttaacggatgcagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggcaaggttgcgcccgccccagcttggtcagccgctccgggcttagaaggccgatctcgcgggcacccagctttcttctacaaagtggccgtttacggatcggctagaatgtcccgcaccggcttaccgatttgcacctcgctaccctgggttcgaccgaagctgaccgatgcttgcgcccgctcgcgaggccggccacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcccggcggatctcgactctagaggatcgctcaggaaggccgctgagatagagccatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgctatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccaacagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcactgatgtctctggaatggaaggaggaggaagtgagcatagcgttggtgcgttgctgtcaagggcgaattgtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgggatccacgatatcctgcaggaattcccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggcatggcggcgcgcagctcttcagtgtctggaacgacacttaagcgtacgtgccggcctggctctccgaaagggcgtattccagcacactggcggccgttactagacccaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattagcttggtcacccggtccgggcctagaaggccagcttcggccgccccgggcaactttattatacaaagttgatagatcgaataacttcgtatagcatacattatacgaagttatcctgagctgattccgatgacttcgtaggttcctagctcaagccgctcgtgtccaagcgtcacttacgattagctaatgattacggcatctaggaccgactagctaactaactagtacgtagaattgtagaattaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccac

SEQ ID NO: 25 - DNA, 21415 bp, Artificial sequence
  Synthetic construct
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttactaccggctggatggcggggccttgatcgtgcaccgccggcgtccggactaactaactagtcgagctagttaccctatgaggtgacatgaagcgctcacggttactatgacggttagcttcacgactgttggtggcagtagcgtacgacttagctatagttccggacttaccgggcccaccggtggtaccgagctcgtttaaacgctcttcaactggaagagcggttaccagagctggtcacctttgtccaccaagatggaactggcgcggctaatttaaatcctgaggatcaactttgtatagaaaagttgggccgaattcgagctcggtacggccagaatggcccggaccgggttaccgaattcgagctcggtaccctgggatccggtgcgggcctcttcgctattacgccagctggcgaaagggggatgtgctgcaaggcgattaagttgggtaacgccagggttttcccagtcacgacgttgtaaaacgacggccagtgccaagctcagatcagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaacaatgccccagttcgacatcctctgcaagaccccccccaaggtgctcgtgaggcagttcgtggagaggttcgagaggccctccggcgagaagatcgccctctgcgccgccgagctcacctacctctgctggatgatcacccacaacggcaccgccattaagagggccaccttcatgtcatacaacaccatcatctccaactccctctccttcgacatcgtgaacaagtccctccagttcaaatacaagacccagaaggccaccatcctcgaggcctccctcaagaagctcatccccgcctgggagttcaccatcatcccctactacggccagaagcaccagtccgacatcaccgacatcgtgtcatccctccagcttcagttcgagtcctccgaggaggctgacaagggcaactcccactccaagaagatgctgaaggccctcctctccgagggcgagtccatctgggagatcaccgagaagatcctcaactccttcgagtacacctccaggttcactaagaccaagaccctctaccagttcctcttcctcgccaccttcatcaactgcggcaggttctcagacatcaagaacgtggaccccaagtccttcaagctcgtgcagaacaagtacctaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtgtcatcatccagtgcctcgtgaccgagaccaagacctccgtgtccaggcacatctacttcttctccgctcgcggcaggatcgaccccctcgtgtacctcgacgagttcctcaggaactcagagcccgtgctcaagagggtgaacaggaccggcaactcctcctccaacaagcaggagtaccagctcctcaaggacaacctcgtgaggtcctacaacaaggccctcaagaagaacgccccctactccatcttcgccatcaagaacggccccaagtcccacatcggtaggcacctcatgacctccttcctctcaatgaagggcctcaccgagctcaccaacgtggtgggcaactggtccgacaagagggcctccgccgtggccaggaccacctacacccaccagatcaccgccatccccgaccactacttcgccctcgtgtcaaggtactacgcctacgaccccatctccaaggagatgatcgccctcaaggacgagactaaccccatcgaggagtggcagcacatcgagcagctcaagggctccgccgagggctccatcaggtaccccgcctggaacggcatcatctcccaggaggtgctcgactacctctcctcctacatcaacaggaggatctgagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgggtaccgagctcgaattcggcccaagtttgtacaaaaaagcaggctccggccagaatggcccggaccgggttaccgaattcgcctagaaggccatttaaatcctgaggatctggtcttcctaaggacccgggatatcggaccgattaaactttaattcggtccgaagcttcgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctgctagcgaagttcctattccgaagttcctattctctagaaagtataggaacttcagatcctagaggatcgatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagatccatgcatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccagcttggtcacccggtccgggcctagaaggccgatctcccgggcacccagctttcttgtacaaagtggccgttaacggatcggccagaatggcccggaccgggttacccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgacgaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggcccatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgaagctgggcaactttattatacaaagttgatagataaatcctgaggatctggtcttcctaaggacccgggatatcggaccgattaaactttaattcggtccgacctggtggcgccgctagcgtatacgaagttcctattccgaagttcctattctccagaaagtataggaacttcatcggaccgattaaactttaattcggtccgacctggtggcgccgctagcataacttcgtatagcatacattatacgaagttatccatatctgaccggcttaccgaatgcgtgctcgctaccctgggatgcgatttcgatggcccctggccgaagctggcatatcgctatgaccgggttagccaattcgacctcgctaccctggggatgctacgtaacgcgtctatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcaccaggcttgggcacccgctccgggcttagaaggccagcttcaagtttgtacaaaaaagcaggcttcgaaggagatagaaccgatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgagctagacccagctttcttgtacaaagtggccgttaacggatgcagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggcaaggttgcgcccgccccagcttggtcagccgctccgggcttagaaggccgatctcgcgggcacccagctttcttctacaaagtggccgtttacggatcggctagaatgtcccgcaccggcttaccgatttgcacctcgctaccctgggttcgaccgaagctgaccgatgcttgcgcccgctcgcgaggccggccaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgctatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccaacagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgggatccacgatatcctgcaggaattcccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggcatggcggcgcgcagctcttcagtgtctggaacgacacttaagcgtacgtgccggcctggctctccgaaagggcgtattccagcacactggcggccgttactagacccaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattagcttggtcacccggtccgggcctagaaggccagcttcggccgccccgggcaactttattatacaaagttgatagatcgaataacttcgtatagcatacattatacgaagttatcctgagctgattccgatgacttcgtaggttcctagctcaagccgctcgtgtccaagcgtcacttacgattagctaatgattacggcatctaggaccgactagctaactaactagtacgtagaattgtagaattaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccac

SEQ ID NO: 26 - DNA, 19946 bp, Artificial Sequence
  Synthetic construct
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttactaccggctggatggcggggccttgatcgtgcaccgccggcgtccggactaactaactagtcgagctagttaccctatgaggtgacatgaagcgctcacggttactatgacggttagcttcacgactgttggtggcagtagcgtacgacttagctatagttccggacttaccgggcccaccggtggtaccgagctcgtttaaacgctcttcaactggaagagcggttaccagagctggtcacctttgtccaccaagatggaactggcgcggctaatttaaatcctgaggatcaactttgtatagaaaagttgggccgaattcgagctcggtacggccagaatggcccggaccgggttaccgaattcgagctcggtaccctgggatccggtgcgggcctcttcgctattacgccagctggcgaaagggggatgtgctgcaaggcgattaagttgggtaacgccagggttttcccagtcacgacgttgtaaaacgacggccagtgccaagctcagatcagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaacaatgccccagttcgacatcctctgcaagaccccccccaaggtgctcgtgaggcagttcgtggagaggttcgagaggccctccggcgagaagatcgccctctgcgccgccgagctcacctacctctgctggatgatcacccacaacggcaccgccattaagagggccaccttcatgtcatacaacaccatcatctccaactccctctccttcgacatcgtgaacaagtccctccagttcaaatacaagacccagaaggccaccatcctcgaggcctccctcaagaagctcatccccgcctgggagttcaccatcatcccctactacggccagaagcaccagtccgacatcaccgacatcgtgtcatccctccagcttcagttcgagtcctccgaggaggctgacaagggcaactcccactccaagaagatgctgaaggccctcctctccgagggcgagtccatctgggagatcaccgagaagatcctcaactccttcgagtacacctccaggttcactaagaccaagaccctctaccagttcctcttcctcgccaccttcatcaactgcggcaggttctcagacatcaagaacgtggaccccaagtccttcaagctcgtgcagaacaagtacctaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtgtcatcatccagtgcctcgtgaccgagaccaagacctccgtgtccaggcacatctacttcttctccgctcgcggcaggatcgaccccctcgtgtacctcgacgagttcctcaggaactcagagcccgtgctcaagagggtgaacaggaccggcaactcctcctccaacaagcaggagtaccagctcctcaaggacaacctcgtgaggtcctacaacaaggccctcaagaagaacgccccctactccatcttcgccatcaagaacggccccaagtcccacatcggtaggcacctcatgacctccttcctctcaatgaagggcctcaccgagctcaccaacgtggtgggcaactggtccgacaagagggcctccgccgtggccaggaccacctacacccaccagatcaccgccatccccgaccactacttcgccctcgtgtcaaggtactacgcctacgaccccatctccaaggagatgatcgccctcaaggacgagactaaccccatcgaggagtggcagcacatcgagcagctcaagggctccgccgagggctccatcaggtaccccgcctggaacggcatcatctcccaggaggtgctcgactacctctcctcctacatcaacaggaggatctgagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgggtaccgagctcgaattcggcccaagtttgtacaaaaaagcaggctccggccagaatggcccggaccgggttaccgaattcgcctagaaggccatttaaatcctgaggatctggtcttcctaaggacccgggatatcggaccgattaaactttaattcggtccgaagcttcgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctgctagcgaagttcctattccgaagttcctattctctagaaagtataggaacttcagatcctagaggatcgatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagatccatgcatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccagcttggtcacccggtccgggcctagaaggccgatctcccgggcacccagctttcttgtacaaagtggccgttaacggatcggccagaatggcccggaccgggttacccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgacgaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggcccatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgaagctgggcaactttattatacaaagttgatagataaatcctgaggatctggtcttcctaaggacccgggatatcggaccgattaaactttaattcggtccgacctggtggcgccgctagcgtatacgaagttcctattccgaagttcctattctccagaaagtataggaacttcatcggaccgattaaactttaattcggtccgacctggtggcgccgctagcataacttcgtatagcatacattatacgaagttatccatatctgaccggcttaccgaatgcgtgctcgctaccctgggatgcgatttcgatggcccctggccgaagctggcatatcgctatgaccgggttagccaattcgacctcgctaccctggggatgctacgtaacgcgtctatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcaccaggcttgggcacccgctccgggcttagaaggccagcttcaagtttgtacaaaaaagcaggcttcgaaggagatagaaccgatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgagctagacccagctttcttgtacaaagtggccgttaacggatgcagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggcaaggttgcgcccgccccagcttggtcagccgctccgggcttagaaggccgatctcgcgggcacccagctttcttctacaaagtggccgtttacggatcggctagaatgtcccgcaccggcttaccgatttgcacctcgctaccctgggttcgaccgaagctgaccgatgcttgcgcccgctcgcgaggccggcctgcagctctagagaaacttcgaaacgcgtggaccgaagcttggttaccgccagaagatagaagatatcctggacctgcaagatgtcagcaatgacgattgaaagattcccaggatagccggcggacgtggtggacccagtctaggtgcgatgcttagtcacgcacgatgactctgtcggaaggcatctttactttcggcaaactttaataatactttaggaaaagtattgtacaagttaggtgcagaatcaataatgcacccagctttagtcttgtctactgaattattgtgtcggttgcattattggatgcctgcgtgcaccctaagcaatccccggctctcatctctataagaggagcctttgtattcagttgcaagcatgcaagtcacacactgcaagcttacttctgagcaaaaagagttttgagtgaaataaatttgaagttcccccttacatcttggatctaggtccgccttgtttctcctctgtctcttgatctgactaatcttggtttatgattcgttgagtaattttggggaaagcttcgtccacagttttttttcgatgaacagtgccgcagtggcgctgatcttgtatgctatcctgcaatcgtggtgaacttatttcttttatatcctttactcccatgaaaaggctagtaatctttctcgatgtaacatcgtccagcactgctattaccgtgtggtccatccgacagtctggctgaacacatcatacgatctatggagcaaaaatctatcttccctgttctttaatgaaggacgtcattttcattagtatgatctaggaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctgaacccgtgcagggatccatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgctatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccaacagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcggcgatcgcggtaccatttaaattgcgcccgccacggccgtggaggtcgtattccggtcagcttgcatccctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggcatggcggcgcgcagctcttcagtgtctggaacgacacttaagcgtacgtgccggcctggctctccgaaagggcgtattccagcacactggcggccgttactagacccaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattagcttggtcacccggtccgggcctagaaggccagcttcggccgccccgggcaactttattatacaaagttgatagatcgaataacttcgtatagcatacattatacgaagttatcctgagctgattccgatgacttcgtaggttcctagctcaagccgctcgtgtccaagcgtcacttacgattagctaatgattacggcatctaggaccgactagctaactaactagtacgtagaattgtagaattaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccac

SEQ ID NO: 27 - DNA, 16348 bp, Artificial sequence
  Synthetic construct
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttactaccggctggatggcggggccttgatcgtgcaccgccggcgtccggactaactaactagtcgagctagttaccctatgaggtgacatgaagcgctcacggttactatgacggttagcttcacgactgttggtggcagtagcgtacgacttagctatagttccggacttaccgggcccaccggtggtaccgagctcgtttaaacgctcttcaactggaagagcggttaccagagctggtcacctttgtccaccaagatggaactggcgcggctaatttaaatcctgaggatcaactttgtatagaaaagttgggccgaattcgagctcggtacggccagaatggcccggaccgggttaccgaattcgagctcggtaccctgggatccggtgcgggcctcttcgctattacgccagctggcgaaagggggatgtgctgcaaggcgattaagttgggtaacgccagggttttcccagtcacgacgttgtaaaacgacggccagtgccaagctcagatcagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaacaatgccccagttcgacatcctctgcaagaccccccccaaggtgctcgtgaggcagttcgtggagaggttcgagaggccctccggcgagaagatcgccctctgcgccgccgagctcacctacctctgctggatgatcacccacaacggcaccgccattaagagggccaccttcatgtcatacaacaccatcatctccaactccctctccttcgacatcgtgaacaagtccctccagttcaaatacaagacccagaaggccaccatcctcgaggcctccctcaagaagctcatccccgcctgggagttcaccatcatcccctactacggccagaagcaccagtccgacatcaccgacatcgtgtcatccctccagcttcagttcgagtcctccgaggaggctgacaagggcaactcccactccaagaagatgctgaaggccctcctctccgagggcgagtccatctgggagatcaccgagaagatcctcaactccttcgagtacacctccaggttcactaagaccaagaccctctaccagttcctcttcctcgccaccttcatcaactgcggcaggttctcagacatcaagaacgtggaccccaagtccttcaagctcgtgcagaacaagtacctaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtgtcatcatccagtgcctcgtgaccgagaccaagacctccgtgtccaggcacatctacttcttctccgctcgcggcaggatcgaccccctcgtgtacctcgacgagttcctcaggaactcagagcccgtgctcaagagggtgaacaggaccggcaactcctcctccaacaagcaggagtaccagctcctcaaggacaacctcgtgaggtcctacaacaaggccctcaagaagaacgccccctactccatcttcgccatcaagaacggccccaagtcccacatcggtaggcacctcatgacctccttcctctcaatgaagggcctcaccgagctcaccaacgtggtgggcaactggtccgacaagagggcctccgccgtggccaggaccacctacacccaccagatcaccgccatccccgaccactacttcgccctcgtgtcaaggtactacgcctacgaccccatctccaaggagatgatcgccctcaaggacgagactaaccccatcgaggagtggcagcacatcgagcagctcaagggctccgccgagggctccatcaggtaccccgcctggaacggcatcatctcccaggaggtgctcgactacctctcctcctacatcaacaggaggatctgagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgggtaccgagctcgaattcggcccaagtttgtacaaaaaagcaggctccggccagaatggcccggaccgggttaccgaattcgcctagaaggccatttaaatcctgaggatctggtcttcctaaggacccgggatatcggaccgattaaactttaattcggtccgaagcttcgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctgctagcgaagttcctattccgaagttcctattctctagaaagtataggaacttcagatcctagaggatcgatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagatccatgcatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccagcttggtcacccggtccgggcctagaaggccgatctcccgggcacccagctttcttgtacaaagtggccgttaacggatcggccagaatggcccggaccgggttacccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgacgaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggcccatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgaagctgggcaactttattatacaaagttgatagataaatcctgaggatctggtcttcctaaggacccgggatatcggaccgattaaactttaattcggtccgacctggtggcgccgctagcgtatacgaagttcctattccgaagttcctattctccagaaagtataggaacttcatcggaccgattaaactttaattcggtccgacctggtggcgccgctagcataacttcgtatagcatacattatacgaagttatccatatctgaccggcttaccgaatgcgtgctcgctaccctgggatgcgatttcgatggcccctggccgaagctggcatatcgctatgaccgggttagccaattcgacctcgctaccctggggatgctacgtaacgcgtctatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcaccaggcttgggcacccgctccgggcttagaaggccagcttcaagtttgtacaaaaaagcaggcttcgaaggagatagaaccgatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgagctagacccagctttcttgtacaaagtggccgttaacggatgcagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggcaaggttgcgcccgccccagcttggtcagccgctccgggcttagaaggccgatctcgcgggcacccagctttcttctacaaagtggccgtttacggatcggctagaatgtcccgcaccggcttaccgatttgcacctcgctaccctgggttcgaccgaagctgaccgatgcttgcgcccgctcgcgaggccggccaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgctatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccaacagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcggcgaggccagcttcggccgccccgggcaactttattatacaaagttgatagatcgaataacttcgtatagcatacattatacgaagttatcctgagctgattccgatgacttcgtaggttcctagctcaagccgctcgtgtccaagcgtcacttacgattagctaatgattacggcatctaggaccgactagctaactaactagtacgtagaattgtagaattaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccac

SEQ ID NO: 28 - DNA, 15324 bp, Artificial sequence
  Synthetic construct
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttactaccggctggatggcggggccttgatcgtgcaccgccggcgtccggactaactaactagtcgagctagttaccctatgaggtgacatgaagcgctcacggttactatgacggttagcttcacgactgttggtggcagtagcgtacgacttagctatagttccggacttaccgggcccaccggtggtaccgagctcgtttaaacgctcttcaactggaagagcggttaccagagctggtcacctttgtccaccaagatggaactggcgcggctaatttaaatcctgaggatcaactttgtatagaaaagttgggccgaattcgagctcggtacggccagaatggcccggaccgggttaccgaattcgagctcggtaccctgggatccggtgcgggcctcttcgctattacgccagctggcgaaagggggatgtgctgcaaggcgattaagttgggtaacgccagggttttcccagtcacgacgttgtaaaacgacggccagtgccaagctcagatcagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaacaatgccccagttcgacatcctctgcaagaccccccccaaggtgctcgtgaggcagttcgtggagaggttcgagaggccctccggcgagaagatcgccctctgcgccgccgagctcacctacctctgctggatgatcacccacaacggcaccgccattaagagggccaccttcatgtcatacaacaccatcatctccaactccctctccttcgacatcgtgaacaagtccctccagttcaaatacaagacccagaaggccaccatcctcgaggcctccctcaagaagctcatccccgcctgggagttcaccatcatcccctactacggccagaagcaccagtccgacatcaccgacatcgtgtcatccctccagcttcagttcgagtcctccgaggaggctgacaagggcaactcccactccaagaagatgctgaaggccctcctctccgagggcgagtccatctgggagatcaccgagaagatcctcaactccttcgagtacacctccaggttcactaagaccaagaccctctaccagttcctcttcctcgccaccttcatcaactgcggcaggttctcagacatcaagaacgtggaccccaagtccttcaagctcgtgcagaacaagtacctaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtgtcatcatccagtgcctcgtgaccgagaccaagacctccgtgtccaggcacatctacttcttctccgctcgcggcaggatcgaccccctcgtgtacctcgacgagttcctcaggaactcagagcccgtgctcaagagggtgaacaggaccggcaactcctcctccaacaagcaggagtaccagctcctcaaggacaacctcgtgaggtcctacaacaaggccctcaagaagaacgccccctactccatcttcgccatcaagaacggccccaagtcccacatcggtaggcacctcatgacctccttcctctcaatgaagggcctcaccgagctcaccaacgtggtgggcaactggtccgacaagagggcctccgccgtggccaggaccacctacacccaccagatcaccgccatccccgaccactacttcgccctcgtgtcaaggtactacgcctacgaccccatctccaaggagatgatcgccctcaaggacgagactaaccccatcgaggagtggcagcacatcgagcagctcaagggctccgccgagggctccatcaggtaccccgcctggaacggcatcatctcccaggaggtgctcgactacctctcctcctacatcaacaggaggatctgagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgggtaccgagctcgaattcggcccaagtttgtacaaaaaagcaggctccggccagaatggcccggaccgggttaccgaattcgcctagaaggccatttaaatcctgaggatctggtcttcctaaggacccgggatatcggaccgattaaactttaattcggtccgaagcttcgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctgctagcgaagttcctattccgaagttcctattctctagaaagtataggaacttcagatcctagaggatcgatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagatccatgcatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccagcttggtcacccggtccgggcctagaaggccgatctcccgggcacccagctttcttgtacaaagtggccgttaacggatcggccagaatggcccggaccgggttacccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgacgaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggcccatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgaagctgggcaactttattatacaaagttgatagataaatcctgaggatctggtcttcctaaggacccgggatatcggaccgattaaactttaattcggtccgacctggtggcgccgctagcgtatacgaagttcctattccgaagttcctattctccagaaagtataggaacttcatcggaccgattaaactttaattcggtccgacctggtggcgccgctagcataacttcgtatagcatacattatacgaagttatccatatctgaccggcttaccgaatgcgtgctcgctaccctgggatgcgatttcgatggcccctggccgaagctggcatatcgctatgaccgggttagccaattcgacctcgctaccctggggatgctacgtaacgcgtctatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcaccaggcttgggcacccgctccgggcttagaaggccagcttcaagtttgtacaaaaaagcaggcttcgaaggagatagaaccgatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgagctagacccagctttcttgtacaaagtggccgttaacggatgcagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggcaaggttgcgcccgccccagcttggtcagccgctccgggcttagaaggccgatctcgcgggcacccagctttcttctacaaagtggccgtttacggatcggctagaatgtcccgcaccggcttaccgatttgcacctcgctaccctgggttcgaccgaagctgaccgatgcttgcgcccgctcgcgaggccggcctgcagctctagagaaacttcgaaacgcgtggaccgaagcttggttaccgccagaagatagaagatatcctggacctgcaagatgtcagcaatgacgattgaaagattcccaggatagccggcggacgtggtggacccagtctaggtgcgatgcttagtcacgcacgatgactctgtcggaaggcatctttactttcggcaaactttaataatactttaggaaaagtattgtacaagttaggtgcagaatcaataatgcacccagctttagtcttgtctactgaattattgtgtcggttgcattattggatgcctgcgtgcaccctaagcaatccccggctctcatctctataagaggagcctttgtattcagttgcaagcatgcaagtcacacactgcaagcttacttctgagcaaaaagagttttgagtgaaataaatttgaagttcccccttacatcttggatctaggtccgccttgtttctcctctgtctcttgatctgactaatcttggtttatgattcgttgagtaattttggggaaagcttcgtccacagttttttttcgatgaacagtgccgcagtggcgctgatcttgtatgctatcctgcaatcgtggtgaacttatttcttttatatcctttactcccatgaaaaggctagtaatctttctcgatgtaacatcgtccagcactgctattaccgtgtggtccatccgacagtctggctgaacacatcatacgatctatggagcaaaaatctatcttccctgttctttaatgaaggacgtcattttcattagtatgatctaggaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctgaacccgtgcagggatccatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgctatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccaacagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcggcgaggccagcttcggccgccccgggcaactttattatacaaagttgatagatcgaataacttcgtatagcatacattatacgaagttatcctgagctgattccgatgacttcgtaggttcctagctcaagccgctcgtgtccaagcgtcacttacgattagctaatgattacggcatctaggaccgactagctaactaactagtacgtagaattgtagaattaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccac

SEQ ID NO: 29 - DNA, 244 bp, Phage P1
atgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgag

SEQ ID NO: 30 - DNA, 785 bp, Phage P1
gtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgac

SEQ ID NO: 31 - DNA, 34 bp, Phage P1
ataacttcgtatagcatacattatacgaagttat

SEQ ID NO: 32 - DNA, 1708 bp, Artificial sequence
  Synthetic construct
acactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcccggcggatctcgactctagaggatcgctcaggaaggccgctgagatagagccatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgctatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccaacagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcactgatgtctctggaatggaaggaggaggaagtgagcatagcgttggtgcgttgctgtcaagggcgaattgtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcg

SEQ ID NO: 33 - DNA, 4531 bp, Artificial sequence
  Synthetic construct
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggcatggcggcgcgcagctcttcagtgtctggaacgacacttaagcgtacgtgccggcctggctctccgaaagggcgtattccagcacactggcggccgttactagacccaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattagctt

SEQ ID NO: 34 - DNA, 48 bp, Saccharomyces cerevisiae
gaagttcctattccgaagttcctattcttcaaaaagtataggaacttc

SEQ ID NO: 35 - DNA, 2616 bp, Artificial sequence
  Synthetic construct
aggcgacccatcgctgctttgtctacatcatgttcttcatcatcctccccaggcgacgcgtgctgctgttcttattcagactaccgttcgagtgactgcatggcgtacatctttctgcatcgactttgtacggctacatcgaacatatacacgagatgtctcgtgtgaatagagtcactaatgccttaagcatcggttactccgtagggtacattctgttcttcttatttgtgcatatttttattgttgtttactgattatacgagtagttatacatacatgcacatacatatcatcacatatatcacaatatttttctaaattaaattaaaactaaaaatgactaaatttctaacaccaacgacattgtaatgttttctccaacaactttacctattctacattgttctatttcgaatttcactctataaacaacatagtctacaatggaaaacagtgctttgtacgactatatacgcgatgtgtggctacaacataagacaatatagtcgtttgaagattgaacctatatatcggtacggttaatccgtctatgtacgtgggcatgacgaacacccgtgataacgaaggattaacgtgcacaatcataaatccaaagtaggagcggtgcatgatgagaatcgctctcagtactcgacataatgaaccttacgaggtacaacaggcaggcaggcagggaccaggggccgcctttatttcaggctcgctggccccacgggcgtgctgcgtgcacgaagggcactaccccaacctctcaccgaaaaccgcgctggatcggcaaatcaaacgaggtggtgccccgtgcccactctccacgtccacggcaccatccctctgcagccgctcaccagccatgccgtgtcgcggaacggcacaaccacccccaacccactcacgaaaccccgtcccggccgtgcccgtgtcggtccgcgctcggcaacgaggcggcccgcgctgctgagtcccctggacacccgacaccctgtcggccctttgtttattcatcccgaaatctcatctgcccccacggccgactgcgctgcgccgcccggatatatatacccatcgttatcgatcgactggggactctatcagtgatagagtctagaggatcgctcaggaaggccgctgagatagaggcatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgccatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccagcagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcactggtaccgttaacagatcattcgacaaagcagcattagtccgttgatcggtggaagaccactcgtcagtgttgagttgaatgtttgatcaataaaatacggcaatgctgtaagggttgttttttatgccattgataatacactgtactgttcagttgttgaactctatttcttagccatgccaagtgcttttcttattttgaataacattacagcaaaaagttgaaagacaaaaaaaaaaacccccgaacagagtgctttgggtcccaagcttctttagactgtgttcggcgttccccctaaatttctccccctatatctcactcacttgtcacatcagcgttctctttccccctatatctccacgctctacagcagttccacctatatcaaacctctataccccaccacaacaatattatatactttcatcttcaactaactcatgtaccttccaatttttttctactaataattatttacgtgcacagaaacttagcaaggagagagagagcgg

SEQ ID NO: 36 - DNA, 4095 bp, Artificial sequence
  Synthetic construct
ccctagctccctgcggctgttacgcggtcccccatcaatcttctgttcttgcggttgtagcctgtgtaacagtgctagagtatgtatgataaataggttttaagtctgcttacatgacattttttattgtggaagagacatataaaaattagagagagtggttctcatgcaacggcggacggcccggtgctaaaagagcttcaagacaaaataatgaaacaggaagagagtagatttatctaagagccaactttattatatgaatgtgtttattgttggctttagatgatatggtaaggagttagagctaataatagataggctctattattattattattaattaaactcgctctaaggaggaaagtgggaggaagggacgaggacgaagactactggaagcatcgtgcatggatgatggatgtggtgtctcttaatgtaggtggccggaggatgtacgtgttaattgcgcgataagcactcagatccaaccgcaaactacctccacactgacacactgatagagagaaagagagacctccgacgactgccgccgcagatgagccacgtacgtatacgacgtctgccggccggctcaggctgccgccatcaccctgctcgaaagtcgcgttaggcggcgccagctacataggagtatctagtctagccagttagtatactactactgcgctgatgatgaattaactctgcatagatactgtacttgcctccctccaacacccaaccacctcctgctcggctcttaataacttggacacggatcgatgccatccaaggaagaacacgacgacgacgacggaacatccaccatgcaagcttgcatccatacgccgatacgcgtgcatccatccatccaccattatttccattttccaccgatcacacgtacacaggcctatttaaggagcgacatcccactgcaactctcctcaccactcatcaccagctagctctagcaaagcacttgccatctaccgaccgccgcattccaaacagcccgacgagctagcagagcggcaggcacctccctcctcaaggaacccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggcatggcggcgcgcagctcttcagtgtctggaacgacacttaagcgtacctagtggtacctgacatcttatagtctgcaacctctcgtgtctgaattcctatctttatcaagtgttattgcttccacgactataggacagctttcgtcgaaagcttttgctcatgtgatctcgaaggattcatctagtctgatttttcgtgacttgtatcggttttattggattcatccaacatatatcaataaaaaatgagttgtgtttcctttcttcctagttcagttaaaattatttccctcctgcgcttgtgctgtaattgtctgtgtacctgttgtttgtgactgtgttagttcccttggatatgatttcgtatttgatatgtacatggagatagcttagcttcattattggagtatgaagttagtatgacatagtcactctcctggaaaattgacactgcaaaccatatttttattctgaaccacaaatcctagtcagtccgctggcatatgccgtccgtttgctgaatccagaacgtgggtttggagatgtacggctgagatgcctctatgcgaaggggatttcgtggtgaaacgagatgggagtagagcaacgcccgtggaagatgcttcaaacttccacacttttgagcaacgatcggcagtagtaaggtagacgatttcaagatcaaagcatatgaagataaacaacatcaacaacaaaatttgttggggttctatagagagaaacagagctacatacatacactgttttgtatctaccatctgagatgatgaaaagatgaaaaactaaagaatgccccggcgccaacgccaggacacgccgcgcgcgcgtcacccgagccatctcttgacccagccggcgctgtatatttacacacgttgcagcatcgatcaccacctgttcgatcgcgtcgccgtcacc

SEQ ID NO: 37 - DNA, 17326 bp, Artificial sequence
  Synthetic construct
gtttacccgccaatatatcctgtcaaacactgatagtttaaactgaaggcgggaaacgacaatctgatcatgagcctagttagttaggggcagctaacgtcccctagctaactagttgctattaacctttagcctagtttagtgcggtcgcgctagtgaactagtctaatgggtgcctagcttagattagatccccgttcggcttagactaggttagtcggatagggctagtctagtggcttagcttggaactgttaagctaggctgttagtgcgggacttctaggctaacctattgacccctaacgcctagaacctaagaaacgctaaggacacctagctaactttggcgacgtccagaggtccgattccggctaactaactagcttaagcttgatatcatgataagaaaacatactagacattaataatctgctagaaatgggaagaatcacgcgtaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccaacaatgccccagttcgacatcctctgcaagaccccccccaaggtgctcgtgaggcagttcgtggagaggttcgagaggccctccggcgagaagatcgccctctgcgccgccgagctcacctacctctgctggatgatcacccacaacggcaccgccattaagagggccaccttcatgtcatacaacaccatcatctccaactccctctccttcgacatcgtgaacaagtccctccagttcaaatacaagacccagaaggccaccatcctcgaggcctccctcaagaagctcatccccgcctgggagttcaccatcatcccctactacggccagaagcaccagtccgacatcaccgacatcgtgtcatccctccagcttcagttcgagtcctccgaggaggctgacaagggcaactcccactccaagaagatgctgaaggccctcctctccgagggcgagtccatctgggagatcaccgagaagatcctcaactccttcgagtacacctccaggttcactaagaccaagaccctctaccagttcctcttcctcgccaccttcatcaactgcggcaggttctcagacatcaagaacgtggaccccaagtccttcaagctcgtgcagaacaagtacctaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtgtcatcatccagtgcctcgtgaccgagaccaagacctccgtgtccaggcacatctacttcttctccgctcgcggcaggatcgaccccctcgtgtacctcgacgagttcctcaggaactcagagcccgtgctcaagagggtgaacaggaccggcaactcctcctccaacaagcaggagtaccagctcctcaaggacaacctcgtgaggtcctacaacaaggccctcaagaagaacgccccctactccatcttcgccatcaagaacggccccaagtcccacatcggtaggcacctcatgacctccttcctctcaatgaagggcctcaccgagctcaccaacgtggtgggcaactggtccgacaagagggcctccgccgtggccaggaccacctacacccaccagatcaccgccatccccgaccactacttcgccctcgtgtcaaggtactacgcctacgaccccatctccaaggagatgatcgccctcaaggacgagactaaccccatcgaggagtggcagcacatcgagcagctcaagggctccgccgagggctccatcaggtaccccgcctggaacggcatcatctcccaggaggtgctcgactacctctcctcctacatcaacaggaggatctgagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcggtcacaccggttaaaaccaaaatccagtggcgagctctcgagtcgatcgctatcaactttgtatagaaaagttgggccgaattcgagctcggtacggccagaatggcccggaccgggttaccgaattcgagctcggtaccctgggatccgatatcgatgggccctggccgaagcttggtcacccggtccgggcctagaaggccagcttcaagtttgtacaaaaaagcaggctccggccagaatggcccggaccgggttaccgaattcgcctagaaggccatttaaatcctgaggatctggtcttcctaaggacccgggatatcggaccgattaaactttaattcggtccgaagcttcgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctgctagcgaagttcctattccgaagttcctattctctagaaagtataggaacttcagatcctagaggatcgatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaaatccatcatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccagcttggtcacaattcaggcgacccatcgctgctttgtctacatcatgttcttcatcatcctccccaggcgacgcgtgctgctgttcttattcagactaccgttcgagtgactgcatggcgtacatctttctgcatcgactttgtacggctacatcgaacatatacacgagatgtctcgtgtgaatagagtcactaatgccttaagcatcggttactccgtagggtacattctgttcttcttatttgtgcatatttttattgttgtttactgattatacgagtagttatacatacatgcacatacatatcatcacatatatcacaatatttttctaaattaaattaaaactaaaaatgactaaatttctaacaccaacgacattgtaatgttttctccaacaactttacctattctacattgttctatttcgaatttcactctataaacaacatagtctacaatggaaaacagtgctttgtacgactatatacgcgatgtgtggctacaacataagacaatatagtcgtttgaagattgaacctatatatcggtacggttaatccgtctatgtacgtgggcatgacgaacacccgtgataacgaaggattaacgtgcacaatcataaatccaaagtaggagcggtgcatgatgagaatcgctctcagtactcgacataatgaaccttacgaggtacaacaggcaggcaggcagggaccaggggccgcctttatttcaggctcgctggccccacgggcgtgctgcgtgcacgaagggcactaccccaacctctcaccgaaaaccgcgctggatcggcaaatcaaacgaggtggtgccccgtgcccactctccacgtccacggcaccatccctctgcagccgctcaccagccatgccgtgtcgcggaacggcacaaccacccccaacccactcacgaaaccccgtcccggccgtgcccgtgtcggtccgcgctcggcaacgaggcggcccgcgctgctgagtcccctggacacccgacaccctgtcggccctttgtttattcatcccgaaatctcatctgcccccacggccgactgcgctgcgccgcccggatatatatacccatcgttatcgatcgactggggactctatcagtgatagagtctagaggatcgctcaggaaggccgctgagatagaggcatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgccatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccagcagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcactggtaccgttaacagatcattcgacaaagcagcattagtccgttgatcggtggaagaccactcgtcagtgttgagttgaatgtttgatcaataaaatacggcaatgctgtaagggttgttttttatgccattgataatacactgtactgttcagttgttgaactctatttcttagccatgccaagtgcttttcttattttgaataacattacagcaaaaagttgaaagacaaaaaaaaaaacccccgaacagagtgctttgggtcccaagcttctttagactgtgttcggcgttccccctaaatttctccccctatatctcactcacttgtcacatcagcgttctctttccccctatatctccacgctctacagcagttccacctatatcaaacctctataccccaccacaacaatattatatactttcatcttcaactaactcatgtaccttccaatttttttctactaataattatttacgtgcacagaaacttagcaaggagagagagagcggggtgacccaccttgctagttggatattacctcttctcttcaaagtatccttgaacgctcaccggttatcaaatctctacactatagctctgtagtcttgctagatagttagttctttagctctcggtgaccaagcttggcgcgatcaagcttatcgataccgtcgacctcgaagcttggtcacccggtccgggcctagaaggccagcttcaagtttgtacaaaaaagcaggctccggccagaatggcccggaccgggttaccgaattcttaccctagctccctgcggctgttacgcggtcccccatcaatcttctgttcttgcggttgtagcctgtgtaacagtgctagagtatgtatgataaataggttttaagtctgcttacatgacattttttattgtggaagagacatataaaaattagagagagtggttctcatgcaacggcggacggcccggtgctaaaagagcttcaagacaaaataatgaaacaggaagagagtagatttatctaagagccaactttattatatgaatgtgtttattgttggctttagatgatatggtaaggagttagagctaataatagataggctctattattattattattaattaaactcgctctaaggaggaaagtgggaggaagggacgaggacgaagactactggaagcatcgtgcatggatgatggatgtggtgtctcttaatgtaggtggccggaggatgtacgtgttaattgcgcgataagcactcagatccaaccgcaaactacctccacactgacacactgatagagagaaagagagacctccgacgactgccgccgcagatgagccacgtacgtatacgacgtctgccggccggctcaggctgccgccatcaccctgctcgaaagtcgcgttaggcggcgccagctacataggagtatctagtctagccagttagtatactactactgcgctgatgatgaattaactctgcatagatactgtacttgcctccctccaacacccaaccacctcctgctcggctcttaataacttggacacggatcgatgccatccaaggaagaacacgacgacgacgacggaacatccaccatgcaagcttgcatccatacgccgatacgcgtgcatccatccatccaccattatttccattttccaccgatcacacgtacacaggcctatttaaggagcgacatcccactgcaactctcctcaccactcatcaccagctagctctagcaaagcacttgccatctaccgaccgccgcattccaaacagcccgacgagctagcagagcggcaggcacctccctcctcaaggaacccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggcatggcggcgcgcagctcttcagtgtctggaacgacacttaagcgtacctagtggtacctgacatcttatagtctgcaacctctcgtgtctgaattcctatctttatcaagtgttattgcttccacgactataggacagctttcgtcgaaagcttttgctcatgtgatctcgaaggattcatctagtctgatttttcgtgacttgtatcggttttattggattcatccaacatatatcaataaaaaatgagttgtgtttcctttcttcctagttcagttaaaattatttccctcctgcgcttgtgctgtaattgtctgtgtacctgttgtttgtgactgtgttagttcccttggatatgatttcgtatttgatatgtacatggagatagcttagcttcattattggagtatgaagttagtatgacatagtcactctcctggaaaattgacactgcaaaccatatttttattctgaaccacaaatcctagtcagtccgctggcatatgccgtccgtttgctgaatccagaacgtgggtttggagatgtacggctgagatgcctctatgcgaaggggatttcgtggtgaaacgagatgggagtagagcaacgcccgtggaagatgcttcaaacttccacacttttgagcaacgatcggcagtagtaaggtagacgatttcaagatcaaagcatatgaagataaacaacatcaacaacaaaatttgttggggttctatagagagaaacagagctacatacatacactgttttgtatctaccatctgagatgatgaaaagatgaaaaactaaagaatgccccggcgccaacgccaggacacgccgcgcgcgcgtcacccgagccatctcttgacccagccggcgctgtatatttacacacgttgcagcatcgatcaccacctgttcgatcgcgtcgccgtcaccggtaccgagctcgaattccggtccgggcctagaaggccgatctcccgggcacccagctttcttgtacaaagtggccgttaacggatcggccagaatggcccggaccgggttacccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgacgaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggcccatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgaagctgggcaactttattatacaaagttgatagatcgagcgactaattaactagctgtcccacggcctaactagcacttaatcccctagcctaacctaagagcgctaatctaggctagtggtcacttagggctttaaggctagcgtatacgaagttcctattccgaagttcctattctccagaaagtataggaacttctgtacacctgagcctaactaactaggacgtcccgaggtccgattccgggctaattaacacctagctcgccactcgactaattagggactaagcctagcgcttagccgttaaaacctagcacaccctaagcacccttagttaggttcccctcttaattaagccctagtgagcccctaagttaaggggacgctaagagccccctaacctagtattcggctagaggcgaactaggctaaacacctaagcgcacctctttaagctagatcgctagggggctaggctagagcttcgctagattagtctaagggcagctaactaactaggtttaaactacattaaaaacgtccgcaatgtgttattaagttgtctaagcgtcaatttgtttacaccacaatatatcctgcca

SEQ ID NO: 38 - DNA, 18003 bp, Artificial sequence
  Synthetic construct
gtttacccgccaatatatcctgtcaaacactgatagtttaaactgaaggcgggaaacgacaatctgatcatgagcctagttagttaggggcagctaacgtcccctagctaactagttgctattaacctttagcctagtttagtgcggtcgcgctagtgaactagtctaatgggtgcctagcttagattagatccccgttcggcttagactaggttagtcggatagggctagtctagtggcttagcttggaactgttaagctaggctgttagtgcgggacttctaggctaacctattgacccctaacgcctagaacctaagaaacgctaaggacacctagctaactttggcgacgtcccgaggtccgattccggctaactaactagcttaagcttgatatcaggcgacccatcgctgctttgtctacatcatgttcttcatcatcctccccaggcgacgcgtgctgctgttcttattcagactaccgttcgagtgactgcatggcgtacatctttctgcatcgactttgtacggctacatcgaacatatacacgagatgtctcgtgtgaatagagtcactaatgccttaagcatcggttactccgtagggtacattctgttcttcttatttgtgcatatttttattgttgtttactgattatacgagtagttatacatacatgcacatacatatcatcacatatatcacaatatttttctaaattaaattaaaactaaaaatgactaaatttctaacaccaacgacattgtaatgttttctccaacaactttacctattctacattgttctatttcgaatttcactctataaacaacatagtctacaatggaaaacagtgctttgtacgactatatacgcgatgtgtggctacaacataagacaatatagtcgtttgaagattgaacctatatatcggtacggttaatccgtctatgtacgtgggcatgacgaacacccgtgataacgaaggattaacgtgcacaatcataaatccaaagtaggagcggtgcatgatgagaatcgctctcagtactcgacataatgaaccttacgaggtacaacaggcaggcaggcagggaccaggggccgcctttatttcaggctcgctggccccacgggcgtgctgcgtgcacgaagggcactaccccaacctctcaccgaaaaccgcgctggatcggcaaatcaaacgaggtggtgccccgtgcccactctccacgtccacggcaccatccctctgcagccgctcaccagccatgccgtgtcgcggaacggcacaaccacccccaacccactcacgaaaccccgtcccggccgtgcccgtgtcggtccgcgctcggcaacgaggcggcccgcgctgctgagtcccctggacacccgacaccctgtcggccctttgtttattcatcccgaaatctcatctgcccccacggccgactgcgctgcgccgcccggatatatatacccatcgttatcgactggggactctatcagtgatagagtctagaggatcgctcaggaaggccgctgagatagaggcatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgctatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccaacagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcactggtaccgttaacagatcaattcgacaaagcagcattagtccgttgatcggtggaagaccactcgtcagtgttgagttgaatgtttgatcaataaaatacggcaatgctgtaagggttgttttttatgccattgataatacactgtactgttcagttgttgaactctatttcttagccatgccaagtgcttttcttattttgaataacattacagcaaaaagttgaaagacaaaaaaaaaaacccccgaacagagtgctttgggtcccaagcttctttagactgtgttcggcgttccccctaaatttctccccctatatctcactcacttgtcacatcagcgttctctttccccctatatctccacgctctacagcagttccacctatatcaaacctctataccccaccacaacaatattatatactttcatcttcaactaactcatgtaccttccaatttttttctactaataattatttacgtgcacagaaacttagcaaggagagagagagcggggtgacccaccttgctagttggatattacctcttctcttcaaagtatccttgaacgctcaccggttatcaaatctctacactatagctctgtagtcttgctagatagttagttctttagctctcggtgaccaagcttggcgcgatcaagcttatcgataccgtcgacctcgaagcttggtcacccggtccgggcctagaaggccagcttcaagtttgtacaaaaaagcaggctccggccagaatggcccggaccgggttaccgaattcttaccctagctccctgcggctgttacgcggtcccccatcaatcttctgttcttgcggttgtagcctgtgtaacagtgctagagtatgtatgataaataggttttaagtctgcttacatgacattttttattgtggaagagacatataaaaattagagagagtggttctcatgcaacggcggacggcccggtgctaaaagagcttcaagacaaaataatgaaacaggaagagagtagatttatctaagagccaactttattatatgaatgtgtttattgttggctttagatgatatggtaaggagttagagctaataatagataggctctattattattattattaattaaactcgctctaaggaggaaagtgggaggaagggacgaggacgaagactactggaagcatcgtgcatggatgatggatgtggtgtctcttaatgtaggtggccggaggatgtacgtgttaattgcgcgataagcactcagatccaaccgcaaactacctccacactgacacactgatagagagaaagagagacctccgacgactgccgccgcagatgagccacgtacgtatacgacgtctgccggccggctcaggctgccgccatcaccctgctcgaaagtcgcgttaggcggcgccagctacataggagtatctagtctagccagttagtatactactactgcgctgatgatgaattaactctgcatagatactgtacttgcctccctccaacacccaaccacctcctgctcggctcttaataacttggacacggatcgatgccatccaaggaagaacacgacgacgacgacggaacatccaccatgcaagcttgcatccatacgccgatacgcgtgcatccatccatccaccattatttccattttccaccgatcacacgtacacaggcctatttaaggagcgacatcccactgcaactctcctcaccactcatcaccagctagctctagcaaagcacttgccatctaccgaccgccgcattccaaacagcccgacgagctagcagagcggcaggcacctccctcctcaaggaacccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggcatggcggcgcgcagctcttcagtgtctggaacgacacttaagcgtacctagtggtacctgacatcttatagtctgcaacctctcgtgtctgaattcctatctttatcaagtgttattgcttccacgactataggacagctttcgtcgaaaggttttgctcatgtgatctcgaaggattcatctagtctgatttttcgtgacttgtatcggttttattggattcatccaacatatatcaataaaaaatgagttgtgtttcctttcttcctagttcagttaaaattatttccctcctgcgcttgtgctgtaattgtctgtgtacctgttgtttgtgactgtgttagttcccttggatatgatttcgtatttgatatgtacatggagatagcttagcttcattattggagtatgaagttagtatgacatagtcactctcctggaaaattgacactgcaaaccatatttttattctgaaccacaaatcctagtcagtccgctggcatatgccgtccgtttgctgaatccagaacgtgggtttggagatgtacggctgagatgcctctatgcgaaggggatttcgtggtgaaacgagatgggagtagagcaacgcccgtggaagatgcttcaaacttccacacttttgagcaacgatcggcagtagtaaggtagacgatttcaagatcaaagcatatgaagataaacaacatcaacaacaaaatttgttggggttctatagagagaaacagagctacatacatacactgttttgtatctaccatctgagatgatgaaaagatgaaaaactaaagaatgccccggcgccaacgccaggacacgccgcgcgcgcgtcacccgagccatctcttgacccagccggcgctgtatatttacacacgttgcagcatcgatcaccacctgttcgatcgcgtcgccgtcacctccggattcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatatcatgataagaaaacatactagacattaataatctgctagaaatgggaagaatcacgcgtaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccaacaatgccccagttcgacatcctctgcaagaccccccccaaggtgctcgtgaggcagttcgtggagaggttcgagaggccctccggcgagaagatcgccctctgcgccgccgagctcacctacctctgctggatgatcacccacaacggcaccgccattaagagggccaccttcatgtcatacaacaccatcatctccaactccctctccttcgacatcgtgaacaagtccctccagttcaaatacaagacccagaaggccaccatcctcgaggcctccctcaagaagctcatccccgcctgggagttcaccatcatcccctactacggccagaagcaccagtccgacatcaccgacatcgtgtcatccctccagcttcagttcgagtcctccgaggaggctgacaagggcaactcccactccaagaagatgctgaaggccctcctctccgagggcgagtccatctgggagatcaccgagaagatcctcaactccttcgagtacacctccaggttcactaagaccaagaccctctaccagttcctcttcctcgccaccttcatcaactgcggcaggttctcagacatcaagaacgtggaccccaagtccttcaagctcgtgcagaacaagtacctaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtgtcatcatccagtgcctcgtgaccgagaccaagacctccgtgtccaggcacatctacttcttctccgctcgcggcaggatcgaccccctcgtgtacctcgacgagttcctcaggaactcagagcccgtgctcaagagggtgaacaggaccggcaactcctcctccaacaagcaggagtaccagctcctcaaggacaacctcgtgaggtcctacaacaaggccctcaagaagaacgccccctactccatcttcgccatcaagaacggccccaagtcccacatcggtaggcacctcatgacctccttcctctcaatgaagggcctcaccgagctcaccaacgtggtgggcaactggtccgacaagagggcctccgccgtggccaggaccacctacacccaccagatcaccgccatccccgaccactacttcgccctcgtgtcaaggtactacgcctacgaccccatctccaaggagatgatcgccctcaaggacgagactaaccccatcgaggagtggcagcacatcgagcagctcaagggctccgccgagggctccatcaggtaccccgcctggaacggcatcatctcccaggaggtgctcgactacctctcctcctacatcaacaggaggatctgagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcggtcacaccggttaaaaccaaaatccagtggcgagctctcgagtcgatcgctatcaactttgtatagaaaagttgggccgaattcgagctcggtacggccagaatggcccggaccgggttaccgaattcgagctcggtaccctgggatccgatatcgatgggccctggccgaagcttggtcacccggtccgggcctagaaggccagcttcaagtttgtacaaaaaagcaggctccggccagaatggcccggaccgggttaccgaattcgcctagaaggccatttaaatcctgaggatctggtcttcctaaggacccgggatatcggaccgattaaactttaattcggtccgaagcttcgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctgctagcgaagttcctattccgaagttcctattctctagaaagtataggaacttcagatcctagaggatcgatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagatccatgcatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccagcttggtcacccggtccgggcctagaaggccgatctcccgggcacccagctttcttgtacaaagtggccgttaacggatcggccagaatggcccggaccgggttacccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgacgaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggcccatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgaagctgggcaactttattatacaaagttgatagatcgagcgactaattaactagctgtcccacggcctaactagcacttaatcccctagcctaacctaagagcgctaatctaggctagtggtcacttagggctttaaggctagcgtatacgaagttcctattccgaagttcctattctccagaaagtataggaacttctgtacacctgagcctaactaactaggacgtcccgaggtccgattccgggctaattaacacctagctcgccactcgactaattagggactaagcctagcgcttagccgttaaaacctagcacaccctaagcacccttagttaggttcccctcttaattaagccctagtgagcccctaagttaaggggacgctaagagccccctaacctagtattcggctagaggcgaactaggctaaacacctaagcgcacctctttaagctagatcgctagggggctaggctagagcttcgctagattagtctaagggcagctaactaactaggtttaaactacattaaaaacgtccgcaatgtgttattaagttgtctaagcgtcaatttgtttacaccacaatatatcctgcca

SEQ ID NO: 39 - DNA, 795 bp, Escherichia coli
atggttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 40 - DNA, 310 bp, Solanum tuberosum
ctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgc

SEQ ID NO: 41 - DNA, 689 bp, Anemonia majano
atggccctgtccaacaagttcatcggcgacgacatgaagatgacctaccacatggacggctgcgtgaacggccactacttcaccgtgaagggcgagggcagcggcaagccctacgagggcacccagacctccaccttcaaggtgaccatggccaacggcggccccctggccttctccttcgacatcctgtccaccgtgttcatgtacggcaaccgctgcttcaccgcctaccccaccagcatgcccgactacttcaagcaggccttccccgacggcatgtcctacgagagaaccttcacctacgaggacggcggcgtggccaccgccagctgggagatcagcctgaagggcaactgcttcgagcacaagtccaccttccacggcgtgaacttccccgccgacggccccgtgatggccaagaagaccaccggctgggacccctccttcgagaagatgaccgtgtgcgacggcatcttgaagggcgacgtgaccgccttcctgatgctgcagggcggcggcaactacagatgccagttccacacctcctacaagaccaagaagcccgtgaccatgccccccaaccacgtggtggagcaccgcatcgccagaaccgacctggacaagggcggcaacagcgtgcagctgaccgagcacgccgtggcccacatcacctccgtggtgcccttctg

SEQ ID NO: 42 - DNA, 1176 bp, Escherichia coli
atgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaa

SEQ ID NO: 43 - DNA, 603 bp, Zea mays
ctatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcacc

(19) United States
(12) Patent Application Publication
(10) Pub. No.: US 2017/0240911 A1
(43) Pub. Date: Aug. 24, 2017

(54) AGROBACTERIUM-MEDIATED SITE SPECIFIC INTEGRATION

(71) Applicant: PIONEER HI-BRED INTERNATIONAL, INC., JOHNSTON, IA (US)

(72) Inventors: AJITH ANAND, WEST DES MOINES, IA (US); WILLIAM JAMES GORDON-KAMM, URBANDALE, IA (US); XINLI EMILY WU, JOHNSTON, IA (US)

(73) Assignee: PIONEER HI-BRED INTERNATIONAL, INC., JOHNSTON, IA (US)

(21) Appl. No.: 15/249,252

(22) Filed: Aug. 26, 2016

Related U.S. Application Data
(60) Provisional application No. 62/296,639, filed on Feb. 18, 2016.

(51) Int. Cl. C12N 15/82

(52) U.S. Cl. CPC C12N 15/8205

(57) ABSTRACT

Methods and compositions for targeting the insertion of a polynucleotide of interest into a specific chromosomal site in the genome of a plant cell using bacteria-mediated transformation methods are provided. The methods include providing a plant cell with at least one Transfer Cassette comprising in operable linkage a least one polynucleotide construct and at least one recombinase construct, and optionally at least one cell proliferation factor construct. Compositions provided include plant cells, plants or seeds having a polynucleotide of interest inserted at a specific chromosomal site.

### CROSS-REFERENCE TO RELATED APPLICATIONS

This application claims priority to U.S. Provisional Application No. 62/296639, filed Feb. 18, 2016, which is hereby incorporated herein in its entirety by reference.

### FIELD

The disclosure relates to the field of plant molecular biology. In particular, methods and compositions are provided for altering the genome of a plant.

### REFERENCE TO A SEQUENCE LISTING SUBMITTED AS A TEXT FILE VIA EFS-WEB

The official copy of the sequence listing is submitted electronically via EFS-Web as an ASCII formatted sequence listing with a file named 20160826_7111USNP_SeqList.txt, created on Aug.t 23, 2016, and having a size of 422 KB and is filed concurrently with the specification. The sequence listing contained in this ASCII formatted document is part of the specification and is herein incorporated by reference in its entirety.

### BACKGROUND

Recombinant DNA technology has made it possible to insert foreign DNA sequences into the genome of an organism, thus, altering the organism's phenotype. The most commonly used plant transformation methods are Agrobacterium infection and biolistic particle bombardment in which genes of interest are introduced into a random location in a plant genome.

Typically, site-specific integration (SSI) technologies rely on these same particle bombardment and/or Agrobacterium-mediated plant transformation methodologies. In some examples a particle bombardment approach may use co-bombardment of a donor gene and a recombinase gene. Particle bombardment SSI can result in damage to the target tissue, transformed cell and/or at the insertion site, which can impact the recovery of clean recombination-mediated gene exchange (RMCE) events or precise SSI events. These physical stresses, including exposure to high osmoticum, vacuum, and/or particle damage can have an adverse impact on the successful recovery or frequency of the desired target transgenic events. Agrobacterium-mediated plant transformation can deliver a donor construct and a recombinase gene using either a single T-DNA vector or a two T-DNA vector approach. Agrobacterium-mediated plant transformation may be less physically stressful to plant cells resulting in minimal damage to the target tissue, transformed cell and/or stably integrated events. However, Agrobacterium-mediated SSI technology is limited by several factors, including low T-DNA delivery to plant cells (low copies of plasmid DNA can be maintained in an Agrobacterium cell), delivery of single-stranded DNA, and overall lower transformation frequencies resulting in low recovery of precise SSI events. Even though, Agrobacterium-mediated SSI could produce a higher quality of SSI events, due to lower frequencies of transformation and recovery of precise SSI event, the method is considered highly inefficient and impractical, and therefore gene gun delivery has remained the method of choice for SSI in plants. Moreover, there have been no reports demonstrating successful Agrobacterium-mediated SSI in monocot species.

Therefore, methods and compositions to improve Agrobacterium transformation and gene targeting into the genome of plants are needed.

### SUMMARY

Methods and compositions for targeting the insertion of a polynucleotide of interest into a specific chromosomal site in the genome of a plant cell using bacteria-mediated transformation methods are provided. The methods include providing a plant cell with at least one Transfer Cassette comprising in operable linkage a least one polynucleotide construct and at least one recombinase construct, and optionally at least one cell proliferation factor construct. Compositions provided include plant cells, plants or seeds having a polynucleotide of interest inserted at a specific chromosomal site.

In some embodiments an agrobacterium-mediated transformation method is used for targeting the insertion of a polynucleotide of interest to a genomic target locus in a monocot plant cell, the method comprising: introducing into the monocot plant cell a transfer cassette, the transfer cassette flanked by a right border and a left border and comprising between the right border and the left border, in operable linkage: a) a polynucleotide construct comprising the polynucleotide of interest, the polynucleotide construct flanked by a first recombination site and a second non-identical recombination site; and b) a recombinase construct encoding a recombinase; wherein the monocot plant cell comprises the genomic target locus comprising the first recombination site and the second non-identical recombination site, whereby the recombinase recognizes and implements recombination at the first and the second non-identical recombination sites such that the polynucleotide construct is inserted at the target locus. In some embodiments the methods further comprise selecting for integration events with or without the cell proliferation factor and the recombinase. In some embodiments the polynucleotide of interest confers a trait selected from the group consisting of pest resistance, herbicide resistance, stress tolerance, drought tolerance, disease resistance, the ability to produce a chemical, and combinations thereof. In some embodiments the first, the second, the third, the fourth, the fifth, and the sixth recombination site is selected from the group consisting of RS, gix, lox, FRT, rox, an integrase, an invertase, a resolvase, and a chimeric recombinase site. In some embodiments the one or more of the first, the second, the third, the fourth, the fifth, and the sixth recombination sites are FRT sites. In some embodiments the first recombination site and the second non-identical sites are FRT sites. In some embodiments the non-identical first recombination and second sites are selected from the group consisting of FRT1/FRT6, FRT1/FRT5, FRT1/FRT12, and FRT1/FRT87. In some embodiments the the non-identical sites are FRT1/FRT87. In some embodiments the one or more of the first, the second, the third, the fourth, the fifth, and the sixth recombination sites are lox sites. In some embodiments the lox sites are selected from the group consisting of loxP, lox 511, lox 5171, lox 2272, lox 71, lox 66, M2, M3, M7, and M11. In some embodiments the lox site is loxP. In some embodiments the recombinase is selected from the group consisting of FLP, Cre, S-CRE, V-CRE, Dre, SSV1, lambda Int, phi C31 Int, HK022, R, Gin, Tn1721, CinH, ParA, Tn5053, Bxb1, TP907-1, and U153. In some embodiments the recombinase is FLP. In some embodiments the cell proliferation factor is selected from the group consisting of a Lec1 polypeptide, a Knotted1 (Kn1) polypeptide, a WUS/WOX homeobox polypeptide, a Zwille polypeptide, a babyboom (ODP2/BBM) polypeptide, an Aintegumenta polypeptide (ANT), a FUS3 polypeptide, a STM polypeptide, an OSH1 polypeptide, a SbH1 polypeptide, or any combination thereof. In some embodiments the cell proliferation factor is selected from the group consisting of a WUS/WOX homeobox polypeptide, a babyboom polypeptide, and combinations thereof. In some embodiments one or more construct further comprises a regulatory element. In some embodiments the regulatory element is selected from the group consisting of a promoter, an operator, an activator, an enhancer, a ribosomal binding site, an initiation codon, a terminator, and any combinations thereof. In some embodiments the the promoter is selected from the group consisting of a non-inducible promoter, an inducible promoter, a constitutive promoter, a tissue-specific promoter, an organ-specific promoter, a developmentally-regulated promoter, a wound-inducible promoter, a pathogen-inducible promoter, a chemical-regulated promoter, a tissue-preferred promoter, and any combinations thereof. In some embodiments one or more construct further comprises an ancillary sequence. In some embodiments the ancillary sequence is selected from the group consisting of a linker, an adapter, an intron, a restriction site, an insulator, a selectable marker, and any combinations thereof. In some embodiments the plant cell is from a monocot. In some embodiments the monocot is selected from the group consisting of maize, rice, sorghum, wheat, sugarcane and millet. In some embodiments the plant cell is from a dicot. In some embodiments the dicot is selected from the group consisting of canola, Brassica, soybean, sunflower, and cotton. In some embodiments the recombinase-mediated recombination results in about 2 or fewer integration events.

In some embodiments an agrobacterium-mediated transformation method is used for targeting the insertion of a polynucleotide of interest to a genomic target locus in a plant cell, the method comprising: introducing into the plant cell a transfer cassette, the transfer cassette flanked by a right border and a left border and comprising between the right border and the left border, in operable linkage: a) a polynucleotide construct comprising the polynucleotide of interest, the polynucleotide construct flanked by a first recombination site and a second non-identical recombination site; b) a cell proliferation factor construct encoding a cell proliferation factor; and c) a recombinase construct encoding a recombinase; wherein the plant cell comprises the genomic target locus comprising the first recombination site and the second non-identical recombination site; whereby the recombinase recognizes and implements recombination at the first recombination site and the second non-identical recombination site such that the polynucleotide construct is inserted at the target locus. In some embodiments the methods further comprise selecting for integration events with or without the cell proliferation factor and the recombinase. In some embodiments the polynucleotide of interest confers a trait selected from the group consisting of pest resistance, herbicide resistance, stress tolerance, drought tolerance, disease resistance, the ability to produce a chemical, and combinations thereof. In some embodiments the first, the second, the third, the fourth, the fifth, and the sixth recombination site is selected from the group consisting of RS, gix, lox, FRT, rox, an integrase, an invertase, a resolvase, and a chimeric recombinase site. In some embodiments the one or more of the first, the second, the third, the fourth, the fifth, and the sixth recombination sites are FRT sites. In some embodiments the first recombination site and the second non-identical sites are FRT sites. In some embodiments the non-identical first recombination and second sites are selected from the group consisting of FRT1/FRT6, FRT1/FRT5, FRT1/FRT12, and FRT1/FRT87. In some embodiments the the non-identical sites are FRT1/FRT87. In some embodiments the one or more of the first, the second, the third, the fourth, the fifth, and the sixth recombination sites are lox sites. In some embodiments the the lox sites are selected from the group consisting of loxP, lox 511, lox 5171, lox 2272, lox 71, lox 66, M2, M3, M7, and M11. In some embodiments the lox site is loxP. In some embodiments the recombinase is selected from the group consisting of FLP, Cre, S-CRE, V-CRE, Dre, SSV1, lambda Int, phi C31 Int, HK022, R, Gin, Tn1721, CinH, ParA, Tn5053, Bxb1, TP907-1, and U153. In some embodiments the recombinase is FLP. In some embodiments the cell proliferation factor is selected from the group consisting of a Lec1 polypeptide, a Knotted1 (Kn1) polypeptide, a WUS/WOX homeobox polypeptide, a Zwille polypeptide, a babyboom (ODP2/BBM) polypeptide, an Aintegumenta polypeptide (ANT), a FUS3 polypeptide, a STM polypeptide, an OSH1 polypeptide, a SbH1 polypeptide, or any combination thereof. In some embodiments the cell proliferation factor is selected from the group consisting of a WUS/WOX homeobox polypeptide, a babyboom polypeptide, and combinations thereof. In some embodiments one or more construct further comprises a regulatory element. In some embodiments the regulatory element is selected from the group consisting of a promoter, an operator, an activator, an enhancer, a ribosomal binding site, an initiation codon, a terminator, and any combinations thereof. In some embodiments the the promoter is selected from the group consisting of a non-inducible promoter, an inducible promoter, a constitutive promoter, a tissue-specific promoter, an organ-specific promoter, a developmentally-regulated promoter, a wound-inducible promoter, a pathogen-inducible promoter, a chemical-regulated promoter, a tissue-preferred promoter, and any combinations thereof. In some embodiments one or more construct further comprises an ancillary sequence. In some embodiments the ancillary sequence is selected from the group consisting of a linker, an adapter, an intron, a restriction site, an insulator, a selectable marker, and any combinations thereof. In some embodiments the plant cell is from a monocot. In some embodiments the monocot is selected from the group consisting of maize, rice, sorghum, wheat, sugarcane and millet. In some embodiments the plant cell is from a dicot. In some embodiments the dicot is selected from the group consisting of canola, Brassica, soybean, sunflower, and cotton. In some embodiments the recombinase-mediated recombination results in about 2 or fewer integration events.

In some embodiments an agrobacterium-mediated transformation method is used for targeting the insertion of a polynucleotide of interest to a genomic target locus in a monocot plant cell, the method comprising: introducing into the monocot plant cell a transfer cassette, the transfer cassette flanked by a right border and a left border and comprising between the right border and the left border, in operable linkage: a) a polynucleotide construct comprising the polynucleotide of interest, the polynucleotide construct flanked by a first recombination site and a second non-identical recombination site; and b) a recombinase construct encoding a recombinase, wherein the recombinase construct is flanked by a third recombination site and a fourth identical recombination site, wherein the third recombination site and the fourth identical recombination site are non-identical to the first recombination site and to the second non-identical recombination site; wherein the plant cell comprises the genomic target locus comprising the first recombination site and the second non-identical recombination site; whereby the recombinase recognizes and implements recombination at the first recombination site and the second non-identical recombination site such that the polynucleotide construct is inserted at the target locus. In some embodiments the methods further comprise selecting for integration events with or without the cell proliferation factor and the recombinase. In some embodiments the polynucleotide of interest confers a trait selected from the group consisting of pest resistance, herbicide resistance, stress tolerance, drought tolerance, disease resistance, the ability to produce a chemical, and combinations thereof. In some embodiments the first, the second, the third, the fourth, the fifth, and the sixth recombination site is selected from the group consisting of RS, gix, lox, FRT, rox, an integrase, an invertase, a resolvase, and a chimeric recombinase site. In some embodiments the one or more of the first, the second, the third, the fourth, the fifth, and the sixth recombination sites are FRT sites. In some embodiments the first recombination site and the second non-identical sites are FRT sites. In some embodiments the non-identical first recombination and second sites are selected from the group consisting of FRT1/FRT6, FRT1/FRT5, FRT1/FRT12, and FRT1/FRT87. In some embodiments the the non-identical sites are FRT1/FRT87. In some embodiments the one or more of the first, the second, the third, the fourth, the fifth, and the sixth recombination sites are lox sites. In some embodiments the the lox sites are selected from the group consisting of loxP, lox 511, lox 5171, lox 2272, lox 71, lox 66, M2, M3, M7, and M11. In some embodiments the lox site is loxP. In some embodiments the recombinase is selected from the group consisting of FLP, Cre, S-CRE, V-CRE, Dre, SSV1, lambda Int, phi C31 Int, HK022, R, Gin, Tn1721, CinH, ParA, Tn5053, Bxb1, TP907-1, and U153. In some embodiments the recombinase is FLP. In some embodiments the cell proliferation factor is selected from the group consisting of a Lec1 polypeptide, a Knotted1 (Kn1) polypeptide, a WUS/WOX homeobox polypeptide, a Zwille polypeptide, a babyboom (ODP2/BBM) polypeptide, an Aintegumenta polypeptide (ANT), a FUS3 polypeptide, a STM polypeptide, an OSH1 polypeptide, a SbH1 polypeptide, or any combination thereof. In some embodiments the cell proliferation factor is selected from the group consisting of a WUS/WOX homeobox polypeptide, a babyboom polypeptide, and combinations thereof. In some embodiments one or more construct further comprises a regulatory element. In some embodiments the regulatory element is selected from the group consisting of a promoter, an operator, an activator, an enhancer, a ribosomal binding site, an initiation codon, a terminator, and any combinations thereof. In some embodiments the the promoter is selected from the group consisting of a non-inducible promoter, an inducible promoter, a constitutive promoter, a tissue-specific promoter, an organ-specific promoter, a developmentally-regulated promoter, a wound-inducible promoter, a pathogen-inducible promoter, a chemical-regulated promoter, a tissue-preferred promoter, and any combinations thereof. In some embodiments one or more construct further comprises an ancillary sequence. In some embodiments the ancillary sequence is selected from the group consisting of a linker, an adapter, an intron, a restriction site, an insulator, a selectable marker, and any combinations thereof. In some embodiments the plant cell is from a monocot. In some embodiments the monocot is selected from the group consisting of maize, rice, sorghum, wheat, sugarcane and millet. In some embodiments the plant cell is from a dicot. In some embodiments the dicot is selected from the group consisting of canola, Brassica, soybean, sunflower, and cotton. In some embodiments the recombinase-mediated recombination results in about 2 or fewer integration events.

In some embodiments an agrobacterium-mediated transformation method is used for targeting the insertion of a polynucleotide of interest to a genomic target locus in a plant cell, the method comprising: introducing into the plant cell a transfer cassette, the transfer cassette flanked by a right border and a left border and comprising between the right border and the left border, in operable linkage: a) a polynucleotide construct comprising the polynucleotide of interest, the polynucleotide construct flanked by a first recombination site and a second non-identical recombination site; and, b) a cell proliferation factor construct encoding a cell proliferation factor and a recombinase construct encoding a recombinase, the cell proliferation factor construct and the recombinase construct are flanked by a third recombination site and a fourth identical recombination site, wherein the third recombination site and the fourth identical recombination site are non-identical to the first recombination site and the second non-identical recombination site; wherein the plant cell comprises the genomic target locus comprising the first recombination site and the second non-identical recombination site; whereby the recombinase recognizes and implements recombination at the first recombination site and the second non-identical recombination site such that the polynucleotide construct is inserted at the target locus. In some embodiments the methods further comprise selecting for integration events with or without the cell proliferation factor and the recombinase. In some embodiments the polynucleotide of interest confers a trait selected from the group consisting of pest resistance, herbicide resistance, stress tolerance, drought tolerance, disease resistance, the ability to produce a chemical, and combinations thereof. In some embodiments the first, the second, the third, the fourth, the fifth, and the sixth recombination site is selected from the group consisting of RS, gix, lox, FRT, rox, an integrase, an invertase, a resolvase, and a chimeric recombinase site. In some embodiments the one or more of the first, the second, the third, the fourth, the fifth, and the sixth recombination sites are FRT sites. In some embodiments the first recombination site and the second non-identical sites are FRT sites. In some embodiments the non-identical first recombination and second sites are selected from the group consisting of FRT1/FRT6, FRT1/FRT5, FRT1/FRT12, and FRT1/FRT87. In some embodiments the the non-identical sites are FRT1/FRT87. In some embodiments the one or more of the first, the second, the third, the fourth, the fifth, and the sixth recombination sites are lox sites. In some embodiments the the lox sites are selected from the group consisting of loxP, lox 511, lox 5171, lox 2272, lox 71, lox 66, M2, M3, M7, and M11. In some embodiments the lox site is loxP. In some embodiments the recombinase is selected from the group consisting of FLP, Cre, S-CRE, V-CRE, Dre, SSV1, lambda Int, phi C31 Int, HK022, R, Gin, Tn1721, CinH, ParA, Tn5053, Bxb1, TP907-1, and U153. In some embodiments the recombinase is FLP. In some embodiments the cell proliferation factor is selected from the group consisting of a Lec1 polypeptide, a Knotted1 (Kn1) polypeptide, a WUS/WOX homeobox polypeptide, a Zwille polypeptide, a babyboom (ODP2/BBM) polypeptide, an Aintegumenta polypeptide (ANT), a FUS3 polypeptide, a STM polypeptide, an OSH1 polypeptide, a SbH1 polypeptide, or any combination thereof. In some embodiments the cell proliferation factor is selected from the group consisting of a WUS/WOX homeobox polypeptide, a babyboom polypeptide, and combinations thereof. In some embodiments one or more construct further comprises a regulatory element. In some embodiments the regulatory element is selected from the group consisting of a promoter, an operator, an activator, an enhancer, a ribosomal binding site, an initiation codon, a terminator, and any combinations thereof. In some embodiments the the promoter is selected from the group consisting of a non-inducible promoter, an inducible promoter, a constitutive promoter, a tissue-specific promoter, an organ-specific promoter, a developmentally-regulated promoter, a wound-inducible promoter, a pathogen-inducible promoter, a chemical-regulated promoter, a tissue-preferred promoter, and any combinations thereof. In some embodiments one or more construct further comprises an ancillary sequence. In some embodiments the ancillary sequence is selected from the group consisting of a linker, an adapter, an intron, a restriction site, an insulator, a selectable marker, and any combinations thereof. In some embodiments the plant cell is from a monocot. In some embodiments the monocot is selected from the group consisting of maize, rice, sorghum, wheat, sugarcane and millet. In some embodiments the plant cell is from a dicot. In some embodiments the dicot is selected from the group consisting of canola, Brassica, soybean, sunflower, and cotton. In some embodiments the recombinase-mediated recombination results in about 2 or fewer integration events.

In some embodiments an agrobacterium-mediated transformation method is used for targeting the insertion of a polynucleotide of interest to a genomic target locus in a plant cell, the method comprising: introducing into the plant cell a transfer cassette, the transfer cassette flanked by a right border and a left border and comprising between the right border and the left border, in operable linkage: a) a polynucleotide construct comprising the polynucleotide of interest, the polynucleotide construct flanked by a first recombination site and a second non-identical recombination site; b) a first recombinase construct encoding a first recombinase; and, c) a cell proliferation factor construct encoding a cell proliferation factor, and a second recombinase construct encoding a second recombinase, the cell proliferation factor construct and the second recombinase construct flanked by a third recombination site and a fourth identical recombination site, wherein the third recombination site and the fourth identical recombination site are non-identical to the first recombination site and the second non-identical recombination site; wherein the plant cell comprises the genomic target locus comprising the first recombination site and the second non-identical recombination site; whereby the first recombinase recognizes and implements recombination at the first recombination site and the second non-identical recombination site such the polynucleotide construct is inserted at the target locus, and whereby the second recombinase recognizes and implements recombination at the third recombination site and the fourth identical recombination site thereby excising the cell proliferation factor construct and the second recombinase construct. In some embodiments the methods further comprise selecting for integration events with or without the cell proliferation factor and the recombinase. In some embodiments the polynucleotide of interest confers a trait selected from the group consisting of pest resistance, herbicide resistance, stress tolerance, drought tolerance, disease resistance, the ability to produce a chemical, and combinations thereof. In some embodiments the first, the second, the third, the fourth, the fifth, and the sixth recombination site is selected from the group consisting of RS, gix, lox, FRT, rox, an integrase, an invertase, a resolvase, and a chimeric recombinase site. In some embodiments the one or more of the first, the second, the third, the fourth, the fifth, and the sixth recombination sites are FRT sites. In some embodiments the first recombination site and the second non-identical sites are FRT sites. In some embodiments the non-identical first recombination and second sites are selected from the group consisting of FRT1/FRT6, FRT1/FRT5, FRT1/FRT12, and FRT1/FRT87. In some embodiments the the non-identical sites are FRT1/FRT87. In some embodiments the one or more of the first, the second, the third, the fourth, the fifth, and the sixth recombination sites are lox sites. In some embodiments the the lox sites are selected from the group consisting of loxP, lox 511, lox 5171, lox 2272, lox 71, lox 66, M2, M3, M7, and M11. In some embodiments the lox site is loxP. In some embodiments the recombinase is selected from the group consisting of FLP, Cre, S-CRE, V-CRE, Dre, SSV1, lambda Int, phi C31 Int, HK022, R, Gin, Tn1721, CinH, ParA, Tn5053, Bxb1, TP907-1, and U153. In some embodiments the recombinase is FLP. In some embodiments the cell proliferation factor is selected from the group consisting of a Lec1 polypeptide, a Knotted1 (Kn1) polypeptide, a WUS/WOX homeobox polypeptide, a Zwille polypeptide, a babyboom (ODP2/BBM) polypeptide, an Aintegumenta polypeptide (ANT), a FUS3 polypeptide, a STM polypeptide, an OSH1 polypeptide, a SbH1 polypeptide, or any combination thereof. In some embodiments the cell proliferation factor is selected from the group consisting of a WUS/WOX homeobox, a babyboom polypeptide, and combinations thereof. In some embodiments one or more construct further comprises a regulatory element. In some embodiments the regulatory element is selected from the group consisting of a promoter, an operator, an activator, an enhancer, a ribosomal binding site, an initiation codon, a terminator, and any combinations thereof. In some embodiments the the promoter is selected from the group consisting of a non-inducible promoter, an inducible promoter, a constitutive promoter, a tissue-specific promoter, an organ-specific promoter, a developmentally-regulated promoter, a wound-inducible promoter, a pathogen-inducible promoter, a chemical-regulated promoter, a tissue-preferred promoter, and any combinations thereof. In some embodiments one or more construct further comprises an ancillary sequence. In some embodiments the ancillary sequence is selected from the group consisting of a linker, an adapter, an intron, a restriction site, an insulator, a selectable marker, and any combinations thereof. In some embodiments the plant cell is from a monocot. In some embodiments the monocot is selected from the group consisting of maize, rice, sorghum, wheat, sugarcane and millet. In some embodiments the plant cell is from a dicot. In some embodiments the dicot is selected from the group consisting of canola, Brassica, soybean, sunflower, and cotton. In some embodiments the recombinase-mediated recombination results in about 2 or fewer integration events.

In some embodiments an agrobacterium-mediated transformation method is used for targeting the insertion of a polynucleotide of interest to a genomic target locus in a plant cell, the method comprising: introducing into the plant cell a transfer cassette, the transfer cassette flanked by a right border and a left border and comprising between the right border and the left border, in operable linkage: a) a first recombination site adjacent to the right border and a second recombination site adjacent to the left border, wherein the first recombination site is non-identical to the second recombination site; b) a polynucleotide construct comprising the polynucleotide of interest, the polynucleotide construct flanked by the first recombination site and a third recombination site, wherein the third recombination site is non-identical to the first recombination site and the second non-identical recombination site; and, c) a cell proliferation factor construct encoding a cell proliferation factor and a recombinase construct encoding a recombinase, wherein the cell proliferation factor construct and the recombinase construct are flanked by the third recombination site and a fourth recombination site, wherein the fourth recombination site is identical to the third recombination site: wherein the plant cell comprises the genomic target locus comprising the first recombination site and the second non-identical recombination site; whereby the recombinase recognizes and implements recombination at the first recombination site and the second non-identical recombination site such that the polynucleotide construct is inserted at the target locus, and whereby the recombinase recognizes and implements recombination at the third recombination site and the fourth identical recombination site thereby excising the cell proliferation factor construct and the recombinase construct. In some embodiments the methods further comprise selecting for integration events with or without the cell proliferation factor and the recombinase. In some embodiments the polynucleotide of interest confers a trait selected from the group consisting of pest resistance, herbicide resistance, stress tolerance, drought tolerance, disease resistance, the ability to produce a chemical, and combinations thereof. In some embodiments the first, the second, the third, the fourth, the fifth, and the sixth recombination site is selected from the group consisting of RS, gix, lox, FRT, rox, an integrase, an invertase, a resolvase, and a chimeric recombinase site. In some embodiments the one or more of the first, the second, the third, the fourth, the fifth, and the sixth recombination sites are FRT sites. In some embodiments the first recombination site and the second non-identical sites are FRT sites. In some embodiments the non-identical first recombination and second sites are selected from the group consisting of FRT1/FRT6, FRT1/FRT5, FRT1/FRT12, and FRT1/FRT87. In some embodiments the the non-identical sites are FRT1/FRT87. In some embodiments the one or more of the first, the second, the third, the fourth, the fifth, and the sixth recombination sites are lox sites. In some embodiments the the lox sites are selected from the group consisting of loxP, lox 511, lox 5171, lox 2272, lox 71, lox 66, M2, M3, M7, and M11. In some embodiments the lox site is loxP. In some embodiments the recombinase is selected from the group consisting of FLP, Cre, S-CRE, V-CRE, Dre, SSV1, lambda Int, phi C31 Int, HK022, R, Gin, Tn1721, CinH, ParA, Tn5053, Bxb1, TP907-1, and U153. In some embodiments the recombinase is FLP. In some embodiments the cell proliferation factor is selected from the group consisting of a Lec1 polypeptide, a Knotted1 (Kn1) polypeptide, a WUS/WOX homeobox) polypeptide, a Zwille polypeptide, a babyboom (ODP2/BBM) polypeptide, an Aintegumenta polypeptide (ANT), a FUS3 polypeptide, a STM polypeptide, an OSH1 polypeptide, a SbH1 polypeptide, or any combination thereof. In some embodiments the cell proliferation factor is selected from the group consisting of a WUS/WOX homeobox polypeptide, a babyboom polypeptide, and combinations thereof. In some embodiments one or more construct further comprises a regulatory element. In some embodiments the regulatory element is selected from the group consisting of a promoter, an operator, an activator, an enhancer, a ribosomal binding site, an initiation codon, a terminator, and any combinations thereof. In some embodiments the the promoter is selected from the group consisting of a non-inducible promoter, an inducible promoter, a constitutive promoter, a tissue-specific promoter, an organ-specific promoter, a developmentally-regulated promoter, a wound-inducible promoter, a pathogen-inducible promoter, a chemical-regulated promoter, a tissue-preferred promoter, and any combinations thereof. In some embodiments one or more construct further comprises an ancillary sequence. In some embodiments the ancillary sequence is selected from the group consisting of a linker, an adapter, an intron, a restriction site, an insulator, a selectable marker, and any combinations thereof. In some embodiments the plant cell is from a monocot. In some embodiments the monocot is selected from the group consisting of maize, rice, sorghum, wheat, sugarcane and millet. In some embodiments the plant cell is from a dicot. In some embodiments the dicot is selected from the group consisting of canola, Brassica, soybean, sunflower, and cotton. In some embodiments the recombinase-mediated recombination results in about 2 or fewer integration events.

In some embodiments an agrobacterium-mediated transformation method is used for targeting the insertion of a polynucleotide of interest to a genomic target locus in a plant cell, the method comprising: introducing into the plant cell a transfer cassette, the transfer cassette flanked by a right border and a left border and comprising between the right border and the left border, in operable linkage: a) a first recombination site adjacent to the right border and a second recombination site adjacent to the left border, wherein the first recombination site is non-identical to the second recombination site; b) a polynucleotide construct comprising the polynucleotide of interest, the polynucleotide construct flanked by the first recombination site and a third recombination site, wherein the third recombination site is non-identical to the first recombination site and the second non-identical recombination site; c) a cell proliferation factor construct encoding a cell proliferation factor, a first recombinase construct encoding a first recombinase, and a second recombinase construct encoding a second recombinase, wherein the cell proliferation factor construct, the first recombinase construct, and the second recombinase construct are flanked by the third recombination site and a fourth recombination site, wherein the fourth recombination site is identical to the third recombination site; wherein the plant cell comprises the genomic target locus comprising the first recombination site and the second non-identical recombination site; whereby the first recombinase recognizes and implements recombination at the first recombination site and the second non-identical recombination site and the second recombinase recognizes and implements recombination at the third recombination site and the fourth identical recombination site such that the polynucleotide construct comprising the third recombination site is inserted at the target locus. In some embodiments the methods further comprise selecting for integration events with or without the cell proliferation factor and the recombinase. In some embodiments the polynucleotide of interest confers a trait selected from the group consisting of pest resistance, herbicide resistance, stress tolerance, drought tolerance, disease resistance, the ability to produce a chemical, and combinations thereof. In some embodiments the first, the second, the third, the fourth, the fifth, and the sixth recombination site is selected from the group consisting of RS, gix, lox, FRT, rox, an integrase, an invertase, a resolvase, and a chimeric recombinase site. In some embodiments the one or more of the first, the second, the third, the fourth, the fifth, and the sixth recombination sites are FRT sites. In some embodiments the first recombination site and the second non-identical sites are FRT sites. In some embodiments the non-identical first recombination and second sites are selected from the group consisting of FRT1/FRT6, FRT1/FRT5, FRT1/FRT12, and FRT1/FRT87. In some embodiments the the non-identical sites are FRT1/FRT87. In some embodiments the one or more of the first, the second, the third, the fourth, the fifth, and the sixth recombination sites are lox sites. In some embodiments the the lox sites are selected from the group consisting of loxP, lox 511, lox 5171, lox 2272, lox 71, lox 66, M2, M3, M7, and M11. In some embodiments the lox site is loxP. In some embodiments the recombinase is selected from the group consisting of FLP, Cre, S-CRE, V-CRE, Dre, SSV1, lambda Int, phi C31 Int, HK022, R, Gin, Tn1721, CinH, ParA, Tn5053, Bxb1, TP907-1, and U153. In some embodiments the recombinase is FLP. In some embodiments the cell proliferation factor is selected from the group consisting of a Lec1 polypeptide, a Knotted1 (Kn1) polypeptide, a WUS/WOX homeobox polypeptide, a Zwille polypeptide, a babyboom (ODP2/BBM) polypeptide, an Aintegumenta polypeptide (ANT), a FUS3 polypeptide, a STM polypeptide, an OSH1 polypeptide, a SbH1 polypeptide, or any combination thereof. In some embodiments the cell proliferation factor is selected from the group consisting of a WUS/WOX homeobox, a babyboom polypeptide, and combinations thereof. In some embodiments one or more construct further comprises a regulatory element. In some embodiments the regulatory element is selected from the group consisting of a promoter, an operator, an activator, an enhancer, a ribosomal binding site, an initiation codon, a terminator, and any combinations thereof. In some embodiments the the promoter is selected from the group consisting of a non-inducible promoter, an inducible promoter, a constitutive promoter, a tissue-specific promoter, an organ-specific promoter, a developmentally-regulated promoter, a wound-inducible promoter, a pathogen-inducible promoter, a chemical-regulated promoter, a tissue-preferred promoter, and any combinations thereof. In some embodiments one or more construct further comprises an ancillary sequence. In some embodiments the ancillary sequence is selected from the group consisting of a linker, an adapter, an intron, a restriction site, an insulator, a selectable marker, and any combinations thereof. In some embodiments the plant cell is from a monocot. In some embodiments the monocot is selected from the group consisting of maize, rice, sorghum, wheat, sugarcane and millet. In some embodiments the plant cell is from a dicot. In some embodiments the dicot is selected from the group consisting of canola, Brassica, soybean, sunflower, and cotton. In some embodiments the recombinase-mediated recombination results in about 2 or fewer integration events.

In some embodiments an agrobacterium-mediated transformation method is used for targeting the insertion of a polynucleotide of interest to a genomic target locus in a plant cell, the method comprising: introducing into the plant cell a transfer cassette, the transfer cassette flanked by a right border and a left border and comprising between the right border and the left border, in operable linkage: a) a first recombination site adjacent to the right border and a second recombination site adjacent to the left border, wherein the first recombination site is non-identical to the second recombination site; b) a polynucleotide construct comprising the polynucleotide of interest, the polynucleotide construct flanked by a third recombination site and a fourth non-identical recombination site, wherein the third recombination site and the fourth non-identical recombination sites are non-identical to the first recombination site, the second non-identical recombination site, and to each other; c) a cell proliferation factor construct encoding a cell proliferation factor, wherein the cell proliferation factor construct is flanked by the first recombination site and a fifth recombination site, wherein the fifth recombination site is identical to the first recombination site; d) a first recombinase construct encoding a first recombinase, wherein the first recombinase construct is flanked by the third non-identical recombination site and the fifth recombination site; and e) a second recombinase construct encoding a second recombinase, wherein the second recombinase is different from the first recombinase, the second recombinase construct is flanked by the fourth recombination site and a sixth recombination site, wherein the sixth recombination site is identical to the fourth recombination site; wherein the plant cell comprises the genomic target locus comprising the second non-identical recombination site and the third non-identical recombination site; whereby the second recombinase recognizes and implements recombination at the second non-identical recombination site, the third non-identical recombination site, the fourth non-identical recombination site, and the sixth recombination site, and the first recombinase recognizes and implements recombination at the first recombination site and the fifth identical recombination site such that after recombination the target locus comprises the third non-identical recombination site, the polynucleotide construct, the fourth non-identical recombination site, and the second non-identical recombination site all in operable linkage. In some embodiments the methods further comprise selecting for integration events with or without the cell proliferation factor and the recombinase. In some embodiments the polynucleotide of interest confers a trait selected from the group consisting of pest resistance, herbicide resistance, stress tolerance, drought tolerance, disease resistance, the ability to produce a chemical, and combinations thereof. In some embodiments the first, the second, the third, the fourth, the fifth, and the sixth recombination site is selected from the group consisting of RS, gix, lox, FRT, rox, an integrase, an invertase, a resolvase, and a chimeric recombinase site. In some embodiments the one or more of the first, the second, the third, the fourth, the fifth, and the sixth recombination sites are FRT sites. In some embodiments the first recombination site and the second non-identical sites are FRT sites. In some embodiments the non-identical first recombination and second sites are selected from the group consisting of FRT1/FRT6, FRT1/FRT5, FRT1/FRT12, and FRT1/FRT87. In some embodiments the the non-identical sites are FRT1/FRT87. In some embodiments the one or more of the first, the second, the third, the fourth, the fifth, and the sixth recombination sites are lox sites. In some embodiments the the lox sites are selected from the group consisting of loxP, lox 511, lox 5171, lox 2272, lox 71, lox 66, M2, M3, M7, and M11. In some embodiments the lox site is loxP. In some embodiments the recombinase is selected from the group consisting of FLP, Cre, S-CRE, V-CRE, Dre, SSV1, lambda Int, phi C31 Int, HK022, R, Gin, Tn1721, CinH, ParA, Tn5053, Bxb1, TP907-1, and U153. In some embodiments the recombinase is FLP. In some embodiments the cell proliferation factor is selected from the group consisting of a Lec1 polypeptide, a Knotted1 (Kn1) polypeptide, a WUS/WOX homeobox polypeptide, a Zwille polypeptide, a babyboom (ODP2/BBM) polypeptide, an Aintegumenta polypeptide (ANT), a FUS3 polypeptide, a STM polypeptide, an OSH1 polypeptide, a SbH1 polypeptide, or any combination thereof. In some embodiments the cell proliferation factor is selected from the group consisting of a WUS/WOX homeobox polypeptide, a babyboom polypeptide, and combinations thereof. In some embodiments one or more construct further comprises a regulatory element. In some embodiments the regulatory element is selected from the group consisting of a promoter, an operator, an activator, an enhancer, a ribosomal binding site, an initiation codon, a terminator, and any combinations thereof. In some embodiments the the promoter is selected from the group consisting of a non-inducible promoter, an inducible promoter, a constitutive promoter, a tissue-specific promoter, an organ-specific promoter, a developmentally-regulated promoter, a wound-inducible promoter, a pathogen-inducible promoter, a chemical-regulated promoter, a tissue-preferred promoter, and any combinations thereof. In some embodiments one or more construct further comprises an ancillary sequence. In some embodiments the ancillary sequence is selected from the group consisting of a linker, an adapter, an intron, a restriction site, an insulator, a selectable marker, and any combinations thereof. In some embodiments the plant cell is from a monocot. In some embodiments the monocot is selected from the group consisting of maize, rice, sorghum, wheat, sugarcane and millet. In some embodiments the plant cell is from a dicot. In some embodiments the dicot is selected from the group consisting of canola, Brassica, soybean, sunflower, and cotton. In some embodiments the recombinase-mediated recombination results in about 2 or fewer integration events.

In some embodiments an agrobacterium-mediated transformation method is used for targeting the insertion of a polynucleotide of interest to a genomic target locus in a plant cell, the method comprising: introducing into the plant cell a transfer cassette, the transfer cassette flanked by a right border and a left border and comprising between the right border and the left border, in operable linkage: a) a first recombination site adjacent to the right border and a second recombination site adjacent to the left border, wherein the first recombination site is non-identical to the second recombination site; b) a polynucleotide construct comprising the polynucleotide of interest, the polynucleotide construct flanked by a third recombination site and a fourth non-identical recombination site; c) a cell proliferation factor construct encoding a cell proliferation factor and a first recombinase construct encoding a first recombinase, wherein the cell proliferation factor construct and the first recombinase construct are flanked by the first recombination site and a fifth recombination site, wherein the fifth recombination site is identical to the first recombination site; and d) a second recombinase construct encoding a second recombinase, wherein the second recombinase is different than the first recombinase, wherein the second recombinase construct is flanked by the fourth recombination site and a sixth recombination site, wherein the sixth recombination site is identical to the fourth recombination site; wherein the plant cell comprises the genomic target locus comprising the third recombination site and the second non-identical recombination site; whereby the second recombinase recognizes and implements recombination at the second non-identical recombination site and the third recombination site, the first recombinase recognizes and implements recombination at the first recombination site and the fifth identical recombination site, and either the first recombinase or the second recombinase recognizes and implements recombination at the fourth recombination site and the sixth recombination site such that after recombination the target locus comprises the third recombination site, the polynucleotide construct, the fourth non-identical recombination site, and the second non-identical recombination site all in operable linkage. In some embodiments the methods further comprise selecting for integration events with or without the cell proliferation factor and the recombinase. In some embodiments the polynucleotide of interest confers a trait selected from the group consisting of pest resistance, herbicide resistance, stress tolerance, drought tolerance, disease resistance, the ability to produce a chemical, and combinations thereof. In some embodiments the first, the second, the third, the fourth, the fifth, and the sixth recombination site is selected from the group consisting of RS, gix, lox, FRT, rox, an integrase, an invertase, a resolvase, and a chimeric recombinase site. In some embodiments the one or more of the first, the second, the third, the fourth, the fifth, and the sixth recombination sites are FRT sites. In some embodiments the first recombination site and the second non-identical sites are FRT sites. In some embodiments the non-identical first recombination and second sites are selected from the group consisting of FRT1/FRT6, FRT1/FRT5, FRT1/FRT12, and FRT1/FRT87. In some embodiments the the non-identical sites are FRT1/FRT87. In some embodiments the one or more of the first, the second, the third, the fourth, the fifth, and the sixth recombination sites are lox sites. In some embodiments the the lox sites are selected from the group consisting of loxP, lox 511, lox 5171, lox 2272, lox 71, lox 66, M2, M3, M7, and M11. In some embodiments the lox site is loxP. In some embodiments the recombinase is selected from the group consisting of FLP, Cre, S-CRE, V-CRE, Dre, SSV1, lambda Int, phi C31 Int, HK022, R, Gin, Tn1721, CinH, ParA, Tn5053, Bxb1, TP907-1, and U153. In some embodiments the recombinase is FLP. In some embodiments the cell proliferation factor is selected from the group consisting of a Lec1 polypeptide, a Knotted1 (Kn1) polypeptide, a WUS/WOX homeobox polypeptide, a Zwille polypeptide, a babyboom (ODP2/BBM) polypeptide, an Aintegumenta polypeptide (ANT), a FUS3 polypeptide, a STM polypeptide, an OSH1 polypeptide, a SbH1 polypeptide, or any combination thereof. In some embodiments the cell proliferation factor is selected from the group consisting of a WUS/WOX homeobox polypeptide, a babyboom polypeptide, and combinations thereof. In some embodiments one or more construct further comprises a regulatory element. In some embodiments the regulatory element is selected from the group consisting of a promoter, an operator, an activator, an enhancer, a ribosomal binding site, an initiation codon, a terminator, and any combinations thereof. In some embodiments the the promoter is selected from the group consisting of a non-inducible promoter, an inducible promoter, a constitutive promoter, a tissue-specific promoter, an organ-specific promoter, a developmentally-regulated promoter, a wound-inducible promoter, a pathogen-inducible promoter, a chemical-regulated promoter, a tissue-preferred promoter, and any combinations thereof. In some embodiments one or more construct further comprises an ancillary sequence. In some embodiments the ancillary sequence is selected from the group consisting of a linker, an adapter, an intron, a restriction site, an insulator, a selectable marker, and any combinations thereof. In some embodiments the plant cell is from a monocot. In some embodiments the monocot is selected from the group consisting of maize, rice, sorghum, wheat, sugarcane and millet. In some embodiments the plant cell is from a dicot. In some embodiments the dicot is selected from the group consisting of canola, Brassica, soybean, sunflower, and cotton. In some embodiments the recombinase-mediated recombination results in about 2 or fewer integration events.

In some embodiments an agrobacterium-mediated transformation method is used for targeting the insertion of a polynucleotide of interest to a genomic target locus in a monocot plant cell, said method comprising: introducing into the monocot plant cell a first transfer cassette and a second transfer cassette, each of the first transfer cassette and the second transfer cassette flanked by a right border and a left border and comprising between each right border and each left border, in operable linkage: a) the first transfer cassette comprising a polynucleotide construct comprising the polynucleotide of interest, wherein the polynucleotide construct is flanked by a first recombination site and a second non-identical recombination site; and, b) the second transfer cassette comprising a recombinase construct, the recombinase construct encoding a recombinase; wherein the monocot plant cell comprises the genomic target locus comprising the first recombination site and the second non-identical recombination site, whereby the recombinase recognizes and implements recombination at the first recombination site and the second non-identical recombination site such that the polynucleotide construct is inserted at the target locus. In some embodiments the methods further comprise selecting for integration events with or without the cell proliferation factor and the recombinase. In some embodiments the polynucleotide of interest confers a trait selected from the group consisting of pest resistance, herbicide resistance, stress tolerance, drought tolerance, disease resistance, the ability to produce a chemical, and combinations thereof. In some embodiments the first, the second, the third, the fourth, the fifth, and the sixth recombination site is selected from the group consisting of RS, gix, lox, FRT, rox, an integrase, an invertase, a resolvase, and a chimeric recombinase site. In some embodiments the one or more of the first, the second, the third, the fourth, the fifth, and the sixth recombination sites are FRT sites. In some embodiments the first recombination site and the second non-identical sites are FRT sites. In some embodiments the non-identical first recombination and second sites are selected from the group consisting of FRT1/FRT6, FRT1/FRT5, FRT1/FRT12, and FRT1/FRT87. In some embodiments the the non-identical sites are FRT1/FRT87. In some embodiments the one or more of the first, the second, the third, the fourth, the fifth, and the sixth recombination sites are lox sites. In some embodiments the the lox sites are selected from the group consisting of loxP, lox 511, lox 5171, lox 2272, lox 71, lox 66, M2, M3, M7, and M11. In some embodiments the lox site is loxP. In some embodiments the recombinase is selected from the group consisting of FLP, Cre, S-CRE, V-CRE, Dre, SSV1, lambda Int, phi C31 Int, HK022, R, Gin, Tn1721, CinH, ParA, Tn5053, Bxb1, TP907-1, and U153. In some embodiments the recombinase is FLP. In some embodiments the cell proliferation factor is selected from the group consisting of a Lec1 polypeptide, a Knotted1 (Kn1) polypeptide, a WUS/WOX homeobox polypeptide, a Zwille polypeptide, a babyboom (ODP2/BBM) polypeptide, an Aintegumenta polypeptide (ANT), a FUS3 polypeptide, a STM polypeptide, an OSH1 polypeptide, a SbH1 polypeptide, or any combination thereof. In some embodiments the cell proliferation factor is selected from the group consisting of a WUS/WOX homeobox polypeptide, a babyboom polypeptide, and combinations thereof. In some embodiments one or more construct further comprises a regulatory element. In some embodiments the regulatory element is selected from the group consisting of a promoter, an operator, an activator, an enhancer, a ribosomal binding site, an initiation codon, a terminator, and any combinations thereof. In some embodiments the the promoter is selected from the group consisting of a non-inducible promoter, an inducible promoter, a constitutive promoter, a tissue-specific promoter, an organ-specific promoter, a developmentally-regulated promoter, a wound-inducible promoter, a pathogen-inducible promoter, a chemical-regulated promoter, a tissue-preferred promoter, and any combinations thereof. In some embodiments one or more construct further comprises an ancillary sequence. In some embodiments the ancillary sequence is selected from the group consisting of a linker, an adapter, an intron, a restriction site, an insulator, a selectable marker, and any combinations thereof. In some embodiments the plant cell is from a monocot. In some embodiments the monocot is selected from the group consisting of maize, rice, sorghum, wheat, sugarcane and millet. In some embodiments the plant cell is from a dicot. In some embodiments the dicot is selected from the group consisting of canola, Brassica, soybean, sunflower, and cotton. In some embodiments the recombinase-mediated recombination results in about 2 or fewer integration events.

In some embodiments an agrobacterium-mediated transformation method is used for targeting the insertion of a polynucleotide of interest to a genomic target locus in a plant cell, said method comprising: introducing into the plant cell a first transfer cassette and a second transfer cassette, each of the first transfer cassette and the second transfer cassette flanked by a right border and a left border and comprising between each right border and each left border, in operable linkage: a) the first transfer cassette comprising a polynucleotide construct comprising the polynucleotide of interest, wherein the polynucleotide construct is flanked by a first recombination site and a second non-identical recombination site; and, b) the second transfer cassette comprising a cell proliferation factor construct and a recombinase construct, the cell proliferation factor construct encoding a cell proliferation factor, and the recombinase construct encoding a recombinase; wherein the plant cell comprises the genomic target locus comprising the first recombination site and the second non-identical recombination site; whereby the recombinase recognizes and implements recombination at the first recombination site and the second non-identical recombination site such that the polynucleotide construct is inserted at the target locus. In some embodiments the methods further comprise selecting for integration events with or without the cell proliferation factor and the recombinase. In some embodiments the polynucleotide of interest confers a trait selected from the group consisting of pest resistance, herbicide resistance, stress tolerance, drought tolerance, disease resistance, the ability to produce a chemical, and combinations thereof. In some embodiments the first, the second, the third, the fourth, the fifth, and the sixth recombination site is selected from the group consisting of RS, gix, lox, FRT, rox, an integrase, an invertase, a resolvase, and a chimeric recombinase site. In some embodiments the one or more of the first, the second, the third, the fourth, the fifth, and the sixth recombination sites are FRT sites. In some embodiments the first recombination site and the second non-identical sites are FRT sites. In some embodiments the non-identical first recombination and second sites are selected from the group consisting of FRT1/FRT6, FRT1/FRT5, FRT1/FRT12, and FRT1/FRT87. In some embodiments the the non-identical sites are FRT1/FRT87. In some embodiments the one or more of the first, the second, the third, the fourth, the fifth, and the sixth recombination sites are lox sites. In some embodiments the the lox sites are selected from the group consisting of loxP, lox 511, lox 5171, lox 2272, lox 71, lox 66, M2, M3, M7, and M11. In some embodiments the lox site is loxP. In some embodiments the recombinase is selected from the group consisting of FLP, Cre, S-CRE, V-CRE, Dre, SSV1, lambda Int, phi C31 Int, HK022, R, Gin, Tn1721, CinH, ParA, Tn5053, Bxb1, TP907-1, and U153. In some embodiments the recombinase is FLP. In some embodiments the cell proliferation factor is selected from the group consisting of a Lec1 polypeptide, a Knotted1 (Kn1) polypeptide, a WUS/WOX homeobox polypeptide, a Zwille polypeptide, a babyboom (ODP2/BBM) polypeptide, an Aintegumenta polypeptide (ANT), a FUS3 polypeptide, a STM polypeptide, an OSH1 polypeptide, a SbH1 polypeptide, or any combination thereof. In some embodiments the cell proliferation factor is selected from the group consisting of a WUS/WOX homeobox polypeptide, a babyboom polypeptide, and combinations thereof. In some embodiments one or more construct further comprises a regulatory element. In some embodiments the regulatory element is selected from the group consisting of a promoter, an operator, an activator, an enhancer, a ribosomal binding site, an initiation codon, a terminator, and any combinations thereof. In some embodiments the the promoter is selected from the group consisting of a non-inducible promoter, an inducible promoter, a constitutive promoter, a tissue-specific promoter, an organ-specific promoter, a developmentally-regulated promoter, a wound-inducible promoter, a pathogen-inducible promoter, a chemical-regulated promoter, a tissue-preferred promoter, and any combinations thereof. In some embodiments one or more construct further comprises an ancillary sequence. In some embodiments the ancillary sequence is selected from the group consisting of a linker, an adapter, an intron, a restriction site, an insulator, a selectable marker, and any combinations thereof. In some embodiments the plant cell is from a monocot. In some embodiments the monocot is selected from the group consisting of maize, rice, sorghum, wheat, sugarcane and millet. In some embodiments the plant cell is from a dicot. In some embodiments the dicot is selected from the group consisting of canola, Brassica, soybean, sunflower, and cotton. In some embodiments the recombinase-mediated recombination results in about 2 or fewer integration events.

In some embodiments an agrobacterium-mediated transformation method is used for targeting the insertion of a polynucleotide of interest to a genomic target locus in a plant cell, said method comprising: introducing into the plant cell a first transfer cassette and a second transfer cassette, each of the first transfer cassette and the second transfer cassette flanked by a right border and a left border and comprising between each right border and each left border, in operable linkage: a) the first transfer cassette comprising a polynucleotide construct and a recombinase construct, wherein the polynucleotide construct comprises the polynucleotide of interest and is flanked by a first recombination site and a second non-identical recombination site, and wherein the recombinase construct encodes a recombinase; and, b) the second transfer cassette comprising a cell proliferation factor construct, the cell proliferation factor construct encoding a cell proliferation factor; wherein the plant cell comprises the genomic target locus comprising the first recombination site and the second non-identical recombination site, whereby the recombinase recognizes and implements recombination at the first recombination site and the second non-identical recombination site such that the polynucleotide construct is inserted at the target locus. In some embodiments the methods further comprise selecting for integration events with or without the cell proliferation factor and the recombinase. In some embodiments the polynucleotide of interest confers a trait selected from the group consisting of pest resistance, herbicide resistance, stress tolerance, drought tolerance, disease resistance, the ability to produce a chemical, and combinations thereof. In some embodiments the first, the second, the third, the fourth, the fifth, and the sixth recombination site is selected from the group consisting of RS, gix, lox, FRT, rox, an integrase, an invertase, a resolvase, and a chimeric recombinase site. In some embodiments the one or more of the first, the second, the third, the fourth, the fifth, and the sixth recombination sites are FRT sites. In some embodiments the first recombination site and the second non-identical sites are FRT sites. In some embodiments the non-identical first recombination and second sites are selected from the group consisting of FRT1/FRT6, FRT1/FRT5, FRT1/FRT12, and FRT1/FRT87. In some embodiments the the non-identical sites are FRT1/FRT87. In some embodiments the one or more of the first, the second, the third, the fourth, the fifth, and the sixth recombination sites are lox sites. In some embodiments the the lox sites are selected from the group consisting of loxP, lox 511, lox 5171, lox 2272, lox 71, lox 66, M2, M3, M7, and M11. In some embodiments the lox site is loxP. In some embodiments the recombinase is selected from the group consisting of FLP, Cre, S-CRE, V-CRE, Dre, SSV1, lambda Int, phi C31 Int, HK022, R, Gin, Tn1721, CinH, ParA, Tn5053, Bxb1, TP907-1, and U153. In some embodiments the recombinase is FLP. In some embodiments the cell proliferation factor is selected from the group consisting of a Lec1 polypeptide, a Knotted1 (Kn1) polypeptide, a WUS/WOX homeobox polypeptide, a Zwille polypeptide, a babyboom (ODP2/BBM) polypeptide, an Aintegumenta polypeptide (ANT), a FUS3 polypeptide, a STM polypeptide, an OSH1 polypeptide, a SbH1 polypeptide, or any combination thereof. In some embodiments the cell proliferation factor is selected from the group consisting of a WUS/WOX homeobox polypeptide, a babyboom polypeptide, and combinations thereof. In some embodiments one or more construct further comprises a regulatory element. In some embodiments the regulatory element is selected from the group consisting of a promoter, an operator, an activator, an enhancer, a ribosomal binding site, an initiation codon, a terminator, and any combinations thereof. In some embodiments the the promoter is selected from the group consisting of a non-inducible promoter, an inducible promoter, a constitutive promoter, a tissue-specific promoter, an organ-specific promoter, a developmentally-regulated promoter, a wound-inducible promoter, a pathogen-inducible promoter, a chemical-regulated promoter, a tissue-preferred promoter, and any combinations thereof. In some embodiments one or more construct further comprises an ancillary sequence. In some embodiments the ancillary sequence is selected from the group consisting of a linker, an adapter, an intron, a restriction site, an insulator, a selectable marker, and any combinations thereof. In some embodiments the plant cell is from a monocot. In some embodiments the monocot is selected from the group consisting of maize, rice, sorghum, wheat, sugarcane and millet. In some embodiments the plant cell is from a dicot. In some embodiments the dicot is selected from the group consisting of canola, Brassica, soybean, sunflower, and cotton. In some embodiments the recombinase-mediated recombination results in about 2 or fewer integration events.

In some embodiments an agrobacterium mediated transformation method is used for targeting the insertion of a polynucleotide of interest to a genomic target locus in a plant cell, said method comprising: introducing into the plant cell a first transfer cassette and a second transfer cassette, each of the first transfer cassette and the second transfer cassette flanked by a right border and a left border and comprising between each right border and each left border, in operable linkage: a) the first transfer cassette comprising a polynucleotide construct comprising the polynucleotide of interest and flanked by a first recombination site and a second non-identical recombination site; and, b) the second transfer cassette comprising a cell proliferation factor construct encoding a cell proliferation factor and a recombinase construct encoding a recombinase, wherein the cell proliferation factor construct is flanked by a third recombination site and a fourth identical recombination site, wherein the third recombination site and the fourth identical recombination site are non-identical to the first recombination site and the second non-identical recombination site; wherein the plant cell comprises the genomic target locus comprising the first recombination site and the second non-identical recombination site; whereby the recombinase recognizes and implements recombination at the first recombination site and the second non-identical recombination site such that the polynucleotide construct is inserted at the target locus. In some embodiments the methods further comprise selecting for integration events with or without the cell proliferation factor and the recombinase. In some embodiments the polynucleotide of interest confers a trait selected from the group consisting of pest resistance, herbicide resistance, stress tolerance, drought tolerance, disease resistance, the ability to produce a chemical, and combinations thereof. In some embodiments the first, the second, the third, the fourth, the fifth, and the sixth recombination site is selected from the group consisting of RS, gix, lox, FRT, rox, an integrase, an invertase, a resolvase, and a chimeric recombinase site. In some embodiments the one or more of the first, the second, the third, the fourth, the fifth, and the sixth recombination sites are FRT sites. In some embodiments the first recombination site and the second non-identical sites are FRT sites. In some embodiments the non-identical first recombination and second sites are selected from the group consisting of FRT1/FRT6, FRT1/FRT5, FRT1/FRT12, and FRT1/FRT87. In some embodiments the the non-identical sites are FRT1/FRT87. In some embodiments the one or more of the first, the second, the third, the fourth, the fifth, and the sixth recombination sites are lox sites. In some embodiments the the lox sites are selected from the group consisting of loxP, lox 511, lox 5171, lox 2272, lox 71, lox 66, M2, M3, M7, and M11. In some embodiments the lox site is loxP. In some embodiments the recombinase is selected from the group consisting of FLP, Cre, S-CRE, V-CRE, Dre, SSV1, lambda Int, phi C31 Int, HK022, R, Gin, Tn1721, CinH, ParA, Tn5053, Bxb1, TP907-1, and U153. In some embodiments the recombinase is FLP. In some embodiments the cell proliferation factor is selected from the group consisting of a Lec1 polypeptide, a Knotted1 (Kn1) polypeptide, a WUS/WOX homeobox polypeptide, a Zwille polypeptide, a babyboom (ODP2/BBM) polypeptide, an Aintegumenta polypeptide (ANT), a FUS3 polypeptide, a STM polypeptide, an OSH1 polypeptide, a SbH1 polypeptide, or any combination thereof. In some embodiments the cell proliferation factor is selected from the group consisting of a WUS/WOX homeobox polypeptide, a babyboom polypeptide, and combinations thereof. In some embodiments one or more construct further comprises a regulatory element. In some embodiments the regulatory element is selected from the group consisting of a promoter, an operator, an activator, an enhancer, a ribosomal binding site, an initiation codon, a terminator, and any combinations thereof. In some embodiments the the promoter is selected from the group consisting of a non-inducible promoter, an inducible promoter, a constitutive promoter, a tissue-specific promoter, an organ-specific promoter, a developmentally-regulated promoter, a wound-inducible promoter, a pathogen-inducible promoter, a chemical-regulated promoter, a tissue-preferred promoter, and any combinations thereof. In some embodiments one or more construct further comprises an ancillary sequence. In some embodiments the ancillary sequence is selected from the group consisting of a linker, an adapter, an intron, a restriction site, an insulator, a selectable marker, and any combinations thereof. In some embodiments the plant cell is from a monocot. In some embodiments the monocot is selected from the group consisting of maize, rice, sorghum, wheat, sugarcane and millet. In some embodiments the plant cell is from a dicot. In some embodiments the dicot is selected from the group consisting of canola, Brassica, soybean, sunflower, and cotton. In some embodiments the recombinase-mediated recombination results in about 2 or fewer integration events.

In some embodiments an agrobacterium mediated transformation method is used for targeting the insertion of a polynucleotide of interest to a genomic target locus in a plant cell, said method comprising: introducing into the plant cell a first transfer cassette and a second transfer cassette, each of the first transfer cassette and the second transfer cassette flanked by a right border and a left border and comprising between each right border and each left border, in operable linkage: a) the first transfer cassette comprising a polynucleotide construct comprising the polynucleotide of interest flanked by a first recombination site and a second non-identical recombination site; and b) the second transfer cassette comprising a cell proliferation factor construct encoding a cell proliferation factor and a recombinase construct encoding a recombinase, wherein the cell proliferation factor construct and the recombinase construct are flanked by a third recombination site and a fourth identical recombination site, wherein the third recombination site and the fourth identical recombination site are non-identical to the first recombination site and the second non-identical recombination site; wherein the plant cell comprises the genomic target locus comprising the first recombination site and the second non-identical recombination site, whereby the recombinase recognizes and implements recombination at the first recombination site and the second non-identical recombination site such that the polynucleotide construct is inserted at the target locus. In some embodiments the methods further comprise selecting for integration events with or without the cell proliferation factor and the recombinase. In some embodiments the polynucleotide of interest confers a trait selected from the group consisting of pest resistance, herbicide resistance, stress tolerance, drought tolerance, disease resistance, the ability to produce a chemical, and combinations thereof. In some embodiments the first, the second, the third, the fourth, the fifth, and the sixth recombination site is selected from the group consisting of RS, gix, lox, FRT, rox, an integrase, an invertase, a resolvase, and a chimeric recombinase site. In some embodiments the one or more of the first, the second, the third, the fourth, the fifth, and the sixth recombination sites are FRT sites. In some embodiments the first recombination site and the second non-identical sites are FRT sites. In some embodiments the non-identical first recombination and second sites are selected from the group consisting of FRT1/FRT6, FRT1/FRT5, FRT1/FRT12, and FRT1/FRT87. In some embodiments the the non-identical sites are FRT1/FRT87. In some embodiments the one or more of the first, the second, the third, the fourth, the fifth, and the sixth recombination sites are lox sites. In some embodiments the the lox sites are selected from the group consisting of loxP, lox 511, lox 5171, lox 2272, lox 71, lox 66, M2, M3, M7, and M11. In some embodiments the lox site is loxP. In some embodiments the recombinase is selected from the group consisting of FLP, Cre, S-CRE, V-CRE, Dre, SSV1, lambda Int, phi C31 Int, HK022, R, Gin, Tn1721, CinH, ParA, Tn5053, Bxb1, TP907-1, and U153. In some embodiments the recombinase is FLP. In some embodiments the cell proliferation factor is selected from the group consisting of a Lec1 polypeptide, a Knotted1 (Kn1) polypeptide, a WUS/WOX homeobox polypeptide, a Zwille polypeptide, a babyboom (ODP2/BBM) polypeptide, an Aintegumenta polypeptide (ANT), a FUS3 polypeptide, a STM polypeptide, an OSH1 polypeptide, a SbH1 polypeptide, or any combination thereof. In some embodiments the cell proliferation factor is selected from the group consisting of a WUS/WOX homeobox polypeptide, a babyboom polypeptide, and combinations thereof. In some embodiments one or more construct further comprises a regulatory element. In some embodiments the regulatory element is selected from the group consisting of a promoter, an operator, an activator, an enhancer, a ribosomal binding site, an initiation codon, a terminator, and any combinations thereof. In some embodiments the the promoter is selected from the group consisting of a non-inducible promoter, an inducible promoter, a constitutive promoter, a tissue-specific promoter, an organ-specific promoter, a developmentally-regulated promoter, a wound-inducible promoter, a pathogen-inducible promoter, a chemical-regulated promoter, a tissue-preferred promoter, and any combinations thereof. In some embodiments one or more construct further comprises an ancillary sequence. In some embodiments the ancillary sequence is selected from the group consisting of a linker, an adapter, an intron, a restriction site, an insulator, a selectable marker, and any combinations thereof. In some embodiments the plant cell is from a monocot. In some embodiments the monocot is selected from the group consisting of maize, rice, sorghum, wheat, sugarcane and millet. In some embodiments the plant cell is from a dicot. In some embodiments the dicot is selected from the group consisting of canola, Brassica, soybean, sunflower, and cotton. In some embodiments the recombinase-mediated recombination results in about 2 or fewer integration events.

In some embodiments an agrobacterium mediated transformation method is used for targeting the insertion of a polynucleotide of interest to a genomic target locus in a plant cell, said method comprising: a) introducing into the plant cell a first transfer cassette comprising, in operable linkage, i) a polynucleotide construct comprising the polynucleotide of interest, wherein the polynucleotide construct is flanked by a first recombination site and a second non-identical recombination site; and ii) a first recombinase construct encoding a first recombinase; and, b) introducing into the plant cell a second transfer cassette comprising, in operable linkage; i) a cell proliferation factor construct encoding a cell proliferation factor and flanked by a third recombination site and a fourth identical recombination site, wherein the third recombination site and the fourth identical recombination site are non-identical to the first recombination site and the second non-identical recombination site; and ii) a second recombinase construct encoding a second recombinase, wherein the second recombinase is different than the first recombinase; wherein the plant cell comprises the genomic target locus comprising the first recombination site and the second non-identical recombination site, whereby the first recombinase recognizes and implements recombination at the first recombination site and the second non-identical recombination site such that the polynucleotide construct is inserted at the target locus. In some embodiments the methods further comprise selecting for integration events with or without the cell proliferation factor and the recombinase. In some embodiments the polynucleotide of interest confers a trait selected from the group consisting of pest resistance, herbicide resistance, stress tolerance, drought tolerance, disease resistance, the ability to produce a chemical, and combinations thereof. In some embodiments the first, the second, the third, the fourth, the fifth, and the sixth recombination site is selected from the group consisting of RS, gix, lox, FRT, rox, an integrase, an invertase, a resolvase, and a chimeric recombinase site. In some embodiments the one or more of the first, the second, the third, the fourth, the fifth, and the sixth recombination sites are FRT sites. In some embodiments the first recombination site and the second non-identical sites are FRT sites. In some embodiments the non-identical first recombination and second sites are selected from the group consisting of FRT1/FRT6, FRT1/FRT5, FRT1/FRT12, and FRT1/FRT87. In some embodiments the the non-identical sites are FRT1/FRT87. In some embodiments the one or more of the first, the second, the third, the fourth, the fifth, and the sixth recombination sites are lox sites. In some embodiments the the lox sites are selected from the group consisting of loxP, lox 511, lox 5171, lox 2272, lox 71, lox 66, M2, M3, M7, and M11. In some embodiments the lox site is loxP. In some embodiments the recombinase is selected from the group consisting of FLP, Cre, S-CRE, V-CRE, Dre, SSV1, lambda Int, phi C31 Int, HK022, R, Gin, Tn1721, CinH, ParA, Tn5053, Bxb1, TP907-1, and U153. In some embodiments the recombinase is FLP. In some embodiments the cell proliferation factor is selected from the group consisting of a Lec1 polypeptide, a Knotted1 (Kn1) polypeptide, a WUS/WOX homeobox polypeptide, a Zwille polypeptide, a babyboom (ODP2/BBM) polypeptide, an Aintegumenta polypeptide (ANT), a FUS3 polypeptide, a STM polypeptide, an OSH1 polypeptide, a SbH1 polypeptide, or any combination thereof. In some embodiments the cell proliferation factor is selected from the group consisting of a WUS/WOX homeobox polypeptide, a babyboom polypeptide, and combinations thereof. In some embodiments one or more construct further comprises a regulatory element. In some embodiments the regulatory element is selected from the group consisting of a promoter, an operator, an activator, an enhancer, a ribosomal binding site, an initiation codon, a terminator, and any combinations thereof. In some embodiments the the promoter is selected from the group consisting of a non-inducible promoter, an inducible promoter, a constitutive promoter, a tissue-specific promoter, an organ-specific promoter, a developmentally-regulated promoter, a wound-inducible promoter, a pathogen-inducible promoter, a chemical-regulated promoter, a tissue-preferred promoter, and any combinations thereof. In some embodiments one or more construct further comprises an ancillary sequence. In some embodiments the ancillary sequence is selected from the group consisting of a linker, an adapter, an intron, a restriction site, an insulator, a selectable marker, and any combinations thereof. In some embodiments the plant cell is from a monocot. In some embodiments the monocot is selected from the group consisting of maize, rice, sorghum, wheat, sugarcane and millet. In some embodiments the plant cell is from a dicot. In some embodiments the dicot is selected from the group consisting of canola, Brassica, soybean, sunflower, and cotton. In some embodiments the recombinase-mediated recombination results in about 2 or fewer integration events.

In some embodiments an agrobacterium mediated transformation method is used for targeting the insertion of a polynucleotide of interest to a genomic target locus in a plant cell, said method comprising: a) introducing into the plant cell a first transfer cassette comprising, in operable linkage, i) a polynucleotide construct comprising the polynucleotide of interest, wherein the polynucleotide construct is flanked by a first recombination site and a second non-identical recombination site; and ii) a first recombinase construct encoding a first recombinase; b) introducing into the plant cell a second transfer cassette comprising, in operable linkage; i) a cell proliferation factor construct encoding a cell proliferation factor and a second recombinase construct encoding a second recombinase, wherein the cell proliferation factor construct and the second recombinase construct are flanked by a third recombination site and a fourth identical recombination site, wherein the third recombination site and the fourth identical recombination site are non-identical to the first recombination site and the second non-identical recombination site, and wherein the second recombinase is different than the first recombinase; wherein the plant cell comprises the genomic target locus comprising the first recombination site and the second non-identical recombination site; whereby the first recombinase recognizes and implements recombination at the first recombination site and the second non-identical recombination site such that the polynucleotide construct is inserted at the target locus. In some embodiments the methods further comprise selecting for integration events with or without the cell proliferation factor and the recombinase. In some embodiments the polynucleotide of interest confers a trait selected from the group consisting of pest resistance, herbicide resistance, stress tolerance, drought tolerance, disease resistance, the ability to produce a chemical, and combinations thereof. In some embodiments the first, the second, the third, the fourth, the fifth, and the sixth recombination site is selected from the group consisting of RS, gix, lox, FRT, rox, an integrase, an invertase, a resolvase, and a chimeric recombinase site. In some embodiments the one or more of the first, the second, the third, the fourth, the fifth, and the sixth recombination sites are FRT sites. In some embodiments the first recombination site and the second non-identical sites are FRT sites. In some embodiments the non-identical first recombination and second sites are selected from the group consisting of FRT1/FRT6, FRT1/FRT5, FRT1/FRT12, and FRT1/FRT87. In some embodiments the the non-identical sites are FRT1/FRT87. In some embodiments the one or more of the first, the second, the third, the fourth, the fifth, and the sixth recombination sites are lox sites. In some embodiments the the lox sites are selected from the group consisting of loxP, lox 511, lox 5171, lox 2272, lox 71, lox 66, M2, M3, M7, and M11. In some embodiments the lox site is loxP. In some embodiments the recombinase is selected from the group consisting of FLP, Cre, S-CRE, V-CRE, Dre, SSV1, lambda Int, phi C31 Int, HK022, R, Gin, Tn1721, CinH, ParA, Tn5053, Bxb1, TP907-1, and U153. In some embodiments the recombinase is FLP. In some embodiments the cell proliferation factor is selected from the group consisting of a Lec1 polypeptide, a Knotted1 (Kn1) polypeptide, a WUS/WOX homeobox polypeptide, a Zwille polypeptide, a babyboom (ODP2/BBM) polypeptide, an Aintegumenta polypeptide (ANT), a FUS3 polypeptide, a STM polypeptide, an OSH1 polypeptide, a SbH1 polypeptide, or any combination thereof. In some embodiments the cell proliferation factor is selected from the group consisting of a WUS/WOX homeobox polypeptide, a babyboom polypeptide, and combinations thereof. In some embodiments one or more construct further comprises a regulatory element. In some embodiments the regulatory element is selected from the group consisting of a promoter, an operator, an activator, an enhancer, a ribosomal binding site, an initiation codon, a terminator, and any combinations thereof. In some embodiments the the promoter is selected from the group consisting of a non-inducible promoter, an inducible promoter, a constitutive promoter, a tissue-specific promoter, an organ-specific promoter, a developmentally-regulated promoter, a wound-inducible promoter, a pathogen-inducible promoter, a chemical-regulated promoter, a tissue-preferred promoter, and any combinations thereof. In some embodiments one or more construct further comprises an ancillary sequence. In some embodiments the ancillary sequence is selected from the group consisting of a linker, an adapter, an intron, a restriction site, an insulator, a selectable marker, and any combinations thereof. In some embodiments the plant cell is from a monocot. In some embodiments the monocot is selected from the group consisting of maize, rice, sorghum, wheat, sugarcane and millet. In some embodiments the plant cell is from a dicot. In some embodiments the dicot is selected from the group consisting of canola, Brassica, soybean, sunflower, and cotton. In some embodiments the recombinase-mediated recombination results in about 2 or fewer integration events.

In some embodiments an agrobacterium-mediated transformation method is used for targeting the insertion of a polynucleotide of interest to a genomic target locus in a plant cell, said method comprising: introducing into the plant cell a first transfer cassette and a second transfer cassette, each of the first transfer cassette and the second transfer cassette flanked by a right border and a left border and comprising between each right border and each left border, in operable linkage: a) the first transfer cassette comprising a polynucleotide construct comprising the polynucleotide of interest, wherein the polynucleotide construct is flanked by a first recombination site and a second non-identical recombination site; and b) the second transfer cassette comprising a cell proliferation factor construct encoding a cell proliferation factor, a first recombinase construct encoding a first recombinase, and a second recombinase construct encoding a second recombinase which is different from the first recombinase, wherein the cell proliferation factor construct and the first recombinase construct are flanked by a third recombination site and a fourth identical recombination site, wherein the third recombination site and the fourth identical recombination site are non-identical to the first recombination site and the second non-identical recombination site; wherein the plant cell comprises the genomic target locus comprising the first recombination site and the second non-identical recombination site, whereby the first recombinase recognizes and implements recombination at the first recombination site and the second non-identical recombination site such that the polynucleotide construct is inserted at the target site. In some embodiments the methods further comprise selecting for integration events with or without the cell proliferation factor and the recombinase. In some embodiments the polynucleotide of interest confers a trait selected from the group consisting of pest resistance, herbicide resistance, stress tolerance, drought tolerance, disease resistance, the ability to produce a chemical, and combinations thereof. In some embodiments the first, the second, the third, the fourth, the fifth, and the sixth recombination site is selected from the group consisting of RS, gix, lox, FRT, rox, an integrase, an invertase, a resolvase, and a chimeric recombinase site. In some embodiments the one or more of the first, the second, the third, the fourth, the fifth, and the sixth recombination sites are FRT sites. In some embodiments the first recombination site and the second non-identical sites are FRT sites. In some embodiments the non-identical first recombination and second sites are selected from the group consisting of FRT1/FRT6, FRT1/FRT5, FRT1/FRT12, and FRT1/FRT87. In some embodiments the the non-identical sites are FRT1/FRT87. In some embodiments the one or more of the first, the second, the third, the fourth, the fifth, and the sixth recombination sites are lox sites. In some embodiments the the lox sites are selected from the group consisting of loxP, lox 511, lox 5171, lox 2272, lox 71, lox 66, M2, M3, M7, and M11. In some embodiments the lox site is loxP. In some embodiments the recombinase is selected from the group consisting of FLP, Cre, S-CRE, V-CRE, Dre, SSV1, lambda Int, phi C31 Int, HK022, R, Gin, Tn1721, CinH, ParA, Tn5053, Bxb1, TP907-1, and U153. In some embodiments the recombinase is FLP. In some embodiments the cell proliferation factor is selected from the group consisting of a Lec1 polypeptide, a Knotted1 (Kn1) polypeptide, a WUS/WOX homeobox polypeptide, a Zwille polypeptide, a babyboom (ODP2/BBM) polypeptide, an Aintegumenta polypeptide (ANT), a FUS3 polypeptide, a STM polypeptide, an OSH1 polypeptide, a SbH1 polypeptide, or any combination thereof. In some embodiments the cell proliferation factor is selected from the group consisting of a WUS/WOX homeobox polypeptide, a babyboom polypeptide, and combinations thereof. In some embodiments one or more construct further comprises a regulatory element. In some embodiments the regulatory element is selected from the group consisting of a promoter, an operator, an activator, an enhancer, a ribosomal binding site, an initiation codon, a terminator, and any combinations thereof. In some embodiments the the promoter is selected from the group consisting of a non-inducible promoter, an inducible promoter, a constitutive promoter, a tissue-specific promoter, an organ-specific promoter, a developmentally-regulated promoter, a wound-inducible promoter, a pathogen-inducible promoter, a chemical-regulated promoter, a tissue-preferred promoter, and any combinations thereof. In some embodiments one or more construct further comprises an ancillary sequence. In some embodiments the ancillary sequence is selected from the group consisting of a linker, an adapter, an intron, a restriction site, an insulator, a selectable marker, and any combinations thereof. In some embodiments the plant cell is from a monocot. In some embodiments the monocot is selected from the group consisting of maize, rice, sorghum, wheat, sugarcane and millet. In some embodiments the plant cell is from a dicot. In some embodiments the dicot is selected from the group consisting of canola, Brassica, soybean, sunflower, and cotton. In some embodiments the recombinase-mediated recombination results in about 2 or fewer integration events.

In some embodiments an agrobacterium-mediated transformation method is used for targeting the insertion of a polynucleotide of interest to a genomic target locus in a plant cell, said method comprising: introducing into the plant cell a first transfer cassette and a second transfer cassette, each of the first transfer cassette and the second transfer cassette flanked by a right border and a left border and comprising between each right border and each left border, in operable linkage: a) the first transfer cassette comprising a polynucleotide construct comprising the polynucleotide of interest, wherein the polynucleotide construct is flanked by a first recombination site and a second non-identical recombination site; b) a second transfer cassette comprising a cell proliferation factor construct encoding a cell proliferation factor, a first recombinase construct encoding a first recombinase, and a second recombinase construct encoding a second recombinase different than the first recombinase, wherein the cell proliferation factor construct, the first recombinase construct, and the second recombinase construct are flanked by a third recombination site and a fourth identical recombination site, wherein the third recombination site and the fourth identical recombination site are non-identical to the first recombination site and the second non-identical recombination site; wherein the plant cell comprises the genomic target locus comprising the first recombination site and the second non-identical recombination site; whereby the first recombinase recognizes and implements recombination at the first recombination site and the second non-identical recombination site such that the polynucleotide construct is inserted at the target locus. In some embodiments the methods further comprise selecting for integration events with or without the cell proliferation factor and the recombinase. In some embodiments the polynucleotide of interest confers a trait selected from the group consisting of pest resistance, herbicide resistance, stress tolerance, drought tolerance, disease resistance, the ability to produce a chemical, and combinations thereof. In some embodiments the first, the second, the third, the fourth, the fifth, and the sixth recombination site is selected from the group consisting of RS, gix, lox, FRT, rox, an integrase, an invertase, a resolvase, and a chimeric recombinase site. In some embodiments the one or more of the first, the second, the third, the fourth, the fifth, and the sixth recombination sites are FRT sites. In some embodiments the first recombination site and the second non-identical sites are FRT sites. In some embodiments the non-identical first recombination and second sites are selected from the group consisting of FRT1/FRT6, FRT1/FRT5, FRT1/FRT12, and FRT1/FRT87. In some embodiments the the non-identical sites are FRT1/FRT87. In some embodiments the one or more of the first, the second, the third, the fourth, the fifth, and the sixth recombination sites are lox sites. In some embodiments the the lox sites are selected from the group consisting of loxP, lox 511, lox 5171, lox 2272, lox 71, lox 66, M2, M3, M7, and M11. In some embodiments the lox site is loxP. In some embodiments the recombinase is selected from the group consisting of FLP, Cre, S-CRE, V-CRE, Dre, SSV1, lambda Int, phi C31 Int, HK022, R, Gin, Tn1721, CinH, ParA, Tn5053, Bxb1, TP907-1, and U153. In some embodiments the recombinase is FLP. In some embodiments the cell proliferation factor is selected from the group consisting of a Lec1 polypeptide, a Knotted1 (Kn1) polypeptide, a WUS/WOX homeobox polypeptide, a Zwille polypeptide, a babyboom (ODP2/BBM) polypeptide, an Aintegumenta polypeptide (ANT), a FUS3 polypeptide, a STM polypeptide, an OSH1 polypeptide, a SbH1 polypeptide, or any combination thereof. In some embodiments the cell proliferation factor is selected from the group consisting of a WUS/WOX homeobox polypeptide, a babyboom polypeptide, and combinations thereof. In some embodiments one or more construct further comprises a regulatory element. In some embodiments the regulatory element is selected from the group consisting of a promoter, an operator, an activator, an enhancer, a ribosomal binding site, an initiation codon, a terminator, and any combinations thereof. In some embodiments the the promoter is selected from the group consisting of a non-inducible promoter, an inducible promoter, a constitutive promoter, a tissue-specific promoter, an organ-specific promoter, a developmentally-regulated promoter, a wound-inducible promoter, a pathogen-inducible promoter, a chemical-regulated promoter, a tissue-preferred promoter, and any combinations thereof. In some embodiments one or more construct further comprises an ancillary sequence. In some embodiments the ancillary sequence is selected from the group consisting of a linker, an adapter, an intron, a restriction site, an insulator, a selectable marker, and any combinations thereof. In some embodiments the plant cell is from a monocot. In some embodiments the monocot is selected from the group consisting of maize, rice, sorghum, wheat, sugarcane and millet. In some embodiments the plant cell is from a dicot. In some embodiments the dicot is selected from the group consisting of canola, Brassica, soybean, sunflower, and cotton. In some embodiments the recombinase-mediated recombination results in about 2 or fewer integration events.

In some embodiments an agrobacterium-mediated transformation method is used for targeting the insertion of a polynucleotide of interest to a genomic target locus in a plant cell, said method comprising: introducing into the plant cell a first transfer cassette and a second transfer cassette, each of the first transfer cassette and the second transfer cassette flanked by a right border and a left border and comprising between each right border and each left border, in operable linkage: a) the first transfer cassette comprising a first recombination site adjacent to the right border and a second non-identical recombination site adjacent to the left border, a polynucleotide construct comprising the polynucleotide of interest, wherein the polynucleotide construct is flanked by the first recombination site and a third non-identical recombination site, and a recombinase construct encoding a recombinase and flanked by the third recombination site and a fourth recombination site identical to the third recombination site; and b) the second transfer cassette comprising a cell proliferation factor construct encoding a cell proliferation factor, wherein the cell proliferation construct is flanked by a fifth recombination site and a sixth identical recombination site, wherein the fifth recombination site and the sixth identical recombination site are non-identical to the first recombination site and the second non-identical recombination site, and are identical to, or non-identical to, the third recombination site and the fourth identical recombination sites, and;wherein the plant cell comprises the genomic target locus comprising the first recombination site and the second non-identical recombination site, whereby the recombinase recognizes and implements recombination at the first recombination site and the second non-identical recombination site such that the polynucleotide construct is inserted at the target locus. In some embodiments the methods further comprise selecting for integration events with or without the cell proliferation factor and the recombinase. In some embodiments the polynucleotide of interest confers a trait selected from the group consisting of pest resistance, herbicide resistance, stress tolerance, drought tolerance, disease resistance, the ability to produce a chemical, and combinations thereof. In some embodiments the first, the second, the third, the fourth, the fifth, and the sixth recombination site is selected from the group consisting of RS, gix, lox, FRT, rox, an integrase, an invertase, a resolvase, and a chimeric recombinase site. In some embodiments the one or more of the first, the second, the third, the fourth, the fifth, and the sixth recombination sites are FRT sites. In some embodiments the first recombination site and the second non-identical sites are FRT sites. In some embodiments the non-identical first recombination and second sites are selected from the group consisting of FRT1/FRT6, FRT1/FRT5, FRT1/FRT12, and FRT1/FRT87. In some embodiments the the non-identical sites are FRT1/FRT87. In some embodiments the one or more of the first, the second, the third, the fourth, the fifth, and the sixth recombination sites are lox sites. In some embodiments the the lox sites are selected from the group consisting of loxP, lox 511, lox 5171, lox 2272, lox 71, lox 66, M2, M3, M7, and M11. In some embodiments the lox site is loxP. In some embodiments the recombinase is selected from the group consisting of FLP, Cre, S-CRE, V-CRE, Dre, SSV1, lambda Int, phi C31 Int, HK022, R, Gin, Tn1721, CinH, ParA, Tn5053, Bxb1, TP907-1, and U153. In some embodiments the recombinase is FLP. In some embodiments the cell proliferation factor is selected from the group consisting of a Lec1 polypeptide, a Knotted1 (Kn1) polypeptide, a WUS/WOX homeobox polypeptide, a Zwille polypeptide, a babyboom (ODP2/BBM) polypeptide, an Aintegumenta polypeptide (ANT), a FUS3 polypeptide, a STM polypeptide, an OSH1 polypeptide, a SbH1 polypeptide, or any combination thereof. In some embodiments the cell proliferation factor is selected from the group consisting of a WUS/WOX homeobox polypeptide, a babyboom polypeptide, and combinations thereof. In some embodiments one or more construct further comprises a regulatory element. In some embodiments the regulatory element is selected from the group consisting of a promoter, an operator, an activator, an enhancer, a ribosomal binding site, an initiation codon, a terminator, and any combinations thereof. In some embodiments the the promoter is selected from the group consisting of a non-inducible promoter, an inducible promoter, a constitutive promoter, a tissue-specific promoter, an organ-specific promoter, a developmentally-regulated promoter, a wound-inducible promoter, a pathogen-inducible promoter, a chemical-regulated promoter, a tissue-preferred promoter, and any combinations thereof. In some embodiments one or more construct further comprises an ancillary sequence. In some embodiments the ancillary sequence is selected from the group consisting of a linker, an adapter, an intron, a restriction site, an insulator, a selectable marker, and any combinations thereof. In some embodiments the plant cell is from a monocot. In some embodiments the monocot is selected from the group consisting of maize, rice, sorghum, wheat, sugarcane and millet. In some embodiments the plant cell is from a dicot. In some embodiments the dicot is selected from the group consisting of canola, Brassica, soybean, sunflower, and cotton. In some embodiments the recombinase-mediated recombination results in about 2 or fewer integration events.

In some embodiments an agrobacterium-mediated transformation method is used for targeting the insertion of a polynucleotide of interest to a genomic target locus in a plant cell, the method comprising: a) introducing into the plant cell a first transfer cassette comprising, in operable linkage; a polynucleotide construct comprising the polynucleotide of interest, and a first recombinase construct encoding a first recombinase, wherein the polynucleotide construct and the first recombinase construct are flanked by a first recombination site and a second non-identical recombination site; and b) introducing into the plant cell a second transfer cassette comprising, in operable linkage; a cell proliferation factor construct encoding a cell proliferation factor and a second recombinase construct encoding a second recombinase different from the first recombinase, wherein the cell proliferation factor construct and the second recombinase construct are flanked by a third recombination site and a fourth identical recombination site, wherein the third recombination site and the fourth recombination site are non-identical to the first recombination site and the second non-identical recombination sites; wherein the plant cell comprises the genomic target locus comprising the first recombination site and the second non-identical recombination site; whereby the first recombinase recognizes and implements recombination at the first recombination site and the second non-identical recombination site such that the polynucleotide construct is inserted at the target site. In some embodiments the methods further comprise selecting for integration events with or without the cell proliferation factor and the recombinase. In some embodiments the polynucleotide of interest confers a trait selected from the group consisting of pest resistance, herbicide resistance, stress tolerance, drought tolerance, disease resistance, the ability to produce a chemical, and combinations thereof. In some embodiments the first, the second, the third, the fourth, the fifth, and the sixth recombination site is selected from the group consisting of RS, gix, lox, FRT, rox, an integrase, an invertase, a resolvase, and a chimeric recombinase site. In some embodiments the one or more of the first, the second, the third, the fourth, the fifth, and the sixth recombination sites are FRT sites. In some embodiments the first recombination site and the second non-identical sites are FRT sites. In some embodiments the non-identical first recombination and second sites are selected from the group consisting of FRT1/FRT6, FRT1/FRT5, FRT1/FRT12, and FRT1/FRT87. In some embodiments the the non-identical sites are FRT1/FRT87. In some embodiments the one or more of the first, the second, the third, the fourth, the fifth, and the sixth recombination sites are lox sites. In some embodiments the the lox sites are selected from the group consisting of loxP, lox 511, lox 5171, lox 2272, lox 71, lox 66, M2, M3, M7, and M11. In some embodiments the lox site is loxP. In some embodiments the recombinase is selected from the group consisting of FLP, Cre, S-CRE, V-CRE, Dre, SSV1, lambda Int, phi C31 Int, HK022, R, Gin, Tn1721, CinH, ParA, Tn5053, Bxb1, TP907-1, and U153. In some embodiments the recombinase is FLP. In some embodiments the cell proliferation factor is selected from the group consisting of a Lec1 polypeptide, a Knotted1 (Kn1) polypeptide, a WUS/WOX homeobox polypeptide, a Zwille polypeptide, a babyboom (ODP2/BBM) polypeptide, an Aintegumenta polypeptide (ANT), a FUS3 polypeptide, a STM polypeptide, an OSH1 polypeptide, a SbH1 polypeptide, or any combination thereof. In some embodiments the cell proliferation factor is selected from the group consisting of a WUS/WOX homeobox polypeptide, a babyboom polypeptide, and combinations thereof. In some embodiments one or more construct further comprises a regulatory element. In some embodiments the regulatory element is selected from the group consisting of a promoter, an operator, an activator, an enhancer, a ribosomal binding site, an initiation codon, a terminator, and any combinations thereof. In some embodiments the the promoter is selected from the group consisting of a non-inducible promoter, an inducible promoter, a constitutive promoter, a tissue-specific promoter, an organ-specific promoter, a developmentally-regulated promoter, a wound-inducible promoter, a pathogen-inducible promoter, a chemical-regulated promoter, a tissue-preferred promoter, and any combinations thereof. In some embodiments one or more construct further comprises an ancillary sequence. In some embodiments the ancillary sequence is selected from the group consisting of a linker, an adapter, an intron, a restriction site, an insulator, a selectable marker, and any combinations thereof. In some embodiments the plant cell is from a monocot. In some embodiments the monocot is selected from the group consisting of maize, rice, sorghum, wheat, sugarcane and millet. In some embodiments the plant cell is from a dicot. In some embodiments the dicot is selected from the group consisting of canola, Brassica, soybean, sunflower, and cotton. In some embodiments the recombinase-mediated recombination results in about 2 or fewer integration events.

In some embodiments an agrobacterium-mediated transformation method is used for targeting the insertion of a polynucleotide of interest to a genomic target locus in a plant cell, the method comprising: a) introducing into the plant cell a first transfer cassette comprising, in operable linkage; i) a polynucleotide construct comprising the polynucleotide of interest and flanked by a first recombination site and a second non-identical recombination site; and ii) a first recombinase construct encoding a first recombinase and flanked by a third recombination site and a fourth identical recombination site, wherein the third recombination site and the fourth identical recombination site are non-identical to the first recombination site and the second non-identical recombination site, and wherein the first recombinase construct and flanking third recombination site and fourth identical recombination site are inside the first recombination site and the second non-identical recombination site; and b) introducing into the plant cell a second transfer cassette comprising, in operable linkage; i) a cell proliferation factor construct encoding a cell proliferation factor, and a second recombinase construct encoding a second recombinase different than the first recombinase, wherein the cell proliferation factor construct and the second recombinase construct are flanked by the third recombination site and the fourth identical recombination site; wherein the plant cell comprises the genomic target locus comprising the first recombination site and the second non-identical recombination site; whereby the first recombinase recognizes and implements recombination at the first recombination site and the second non-identical recombination site such that the polynucleotide construct is inserted at the target locus. In some embodiments the methods further comprise selecting for integration events with or without the cell proliferation factor and the recombinase. In some embodiments the polynucleotide of interest confers a trait selected from the group consisting of pest resistance, herbicide resistance, stress tolerance, drought tolerance, disease resistance, the ability to produce a chemical, and combinations thereof. In some embodiments the first, the second, the third, the fourth, the fifth, and the sixth recombination site is selected from the group consisting of RS, gix, lox, FRT, rox, an integrase, an invertase, a resolvase, and a chimeric recombinase site. In some embodiments the one or more of the first, the second, the third, the fourth, the fifth, and the sixth recombination sites are FRT sites. In some embodiments the first recombination site and the second non-identical sites are FRT sites. In some embodiments the non-identical first recombination and second sites are selected from the group consisting of FRT1/FRT6, FRT1/FRT5, FRT1/FRT12, and FRT1/FRT87. In some embodiments the the non-identical sites are FRT1/FRT87. In some embodiments the one or more of the first, the second, the third, the fourth, the fifth, and the sixth recombination sites are lox sites. In some embodiments the the lox sites are selected from the group consisting of loxP, lox 511, lox 5171, lox 2272, lox 71, lox 66, M2, M3, M7, and M11. In some embodiments the lox site is loxP. In some embodiments the recombinase is selected from the group consisting of FLP, Cre, S-CRE, V-CRE, Dre, SSV1, lambda Int, phi C31 Int, HK022, R, Gin, Tn1721, CinH, ParA, Tn5053, Bxb1, TP907-1, and U153. In some embodiments the recombinase is FLP. In some embodiments the cell proliferation factor is selected from the group consisting of a Lec1 polypeptide, a Knotted1 (Kn1) polypeptide, a WUS/WOX homeobox polypeptide, a Zwille polypeptide, a babyboom (ODP2/BBM) polypeptide, an Aintegumenta polypeptide (ANT), a FUS3 polypeptide, a STM polypeptide, an OSH1 polypeptide, a SbH1 polypeptide, or any combination thereof. In some embodiments the cell proliferation factor is selected from the group consisting of a WUS/WOX homeobox polypeptide, a babyboom polypeptide, and combinations thereof. In some embodiments one or more construct further comprises a regulatory element. In some embodiments the regulatory element is selected from the group consisting of a promoter, an operator, an activator, an enhancer, a ribosomal binding site, an initiation codon, a terminator, and any combinations thereof. In some embodiments the the promoter is selected from the group consisting of a non-inducible promoter, an inducible promoter, a constitutive promoter, a tissue-specific promoter, an organ-specific promoter, a developmentally-regulated promoter, a wound-inducible promoter, a pathogen-inducible promoter, a chemical-regulated promoter, a tissue-preferred promoter, and any combinations thereof. In some embodiments one or more construct further comprises an ancillary sequence. In some embodiments the ancillary sequence is selected from the group consisting of a linker, an adapter, an intron, a restriction site, an insulator, a selectable marker, and any combinations thereof. In some embodiments the plant cell is from a monocot. In some embodiments the monocot is selected from the group consisting of maize, rice, sorghum, wheat, sugarcane and millet. In some embodiments the plant cell is from a dicot. In some embodiments the dicot is selected from the group consisting of canola, Brassica, soybean, sunflower, and cotton. In some embodiments the recombinase-mediated recombination results in about 2 or fewer integration events.

In some embodiments the methods further comprise selecting for integration events with or without the cell proliferation factor and the recombinase. In some embodiments the polynucleotide of interest confers a trait selected from the group consisting of pest resistance, herbicide resistance, stress tolerance, drought tolerance, disease resistance, the ability to produce a chemical, and combinations thereof. In some embodiments the first, the second, the third, the fourth, the fifth, and the sixth recombination site is selected from the group consisting of RS, gix, lox, FRT, rox, an integrase, an invertase, a resolvase, and a chimeric recombinase site. In some embodiments the one or more of the first, the second, the third, the fourth, the fifth, and the sixth recombination sites are FRT sites. In some embodiments the first recombination site and the second non-identical sites are FRT sites. In some embodiments the non-identical first recombination and second sites are selected from the group consisting of FRT1/FRT6, FRT1/FRT5, FRT1/FRT12, and FRT1/FRT87. In some embodiments the the non-identical sites are FRT1/FRT87. In some embodiments the one or more of the first, the second, the third, the fourth, the fifth, and the sixth recombination sites are lox sites. In some embodiments the the lox sites are selected from the group consisting of loxP, lox 511, lox 5171, lox 2272, lox 71, lox 66, M2, M3, M7, and M11. In some embodiments the lox site is loxP. In some embodiments the recombinase is selected from the group consisting of FLP, Cre, S-CRE, V-CRE, Dre, SSV1, lambda Int, phi C31 Int, HK022, R, Gin, Tn1721, CinH, ParA, Tn5053, Bxb1, TP907-1, and U153. In some embodiments the recombinase is FLP. In some embodiments the cell proliferation factor is selected from the group consisting of a Lec1 polypeptide, a Knotted1 (Kn1) polypeptide, a WUS/WOX homeobox polypeptide, a Zwille polypeptide, a babyboom (ODP2/BBM) polypeptide, an Aintegumenta polypeptide (ANT), a FUS3 polypeptide, a STM polypeptide, an OSH1 polypeptide, a SbH1 polypeptide, or any combination thereof. In some embodiments the cell proliferation factor is selected from the group consisting of a WUS/WOX homeobox polypeptide, a babyboom polypeptide, and combinations thereof. In some embodiments one or more construct further comprises a regulatory element. In some embodiments the regulatory element is selected from the group consisting of a promoter, an operator, an activator, an enhancer, a ribosomal binding site, an initiation codon, a terminator, and any combinations thereof. In some embodiments the the promoter is selected from the group consisting of a non-inducible promoter, an inducible promoter, a constitutive promoter, a tissue-specific promoter, an organ-specific promoter, a developmentally-regulated promoter, a wound-inducible promoter, a pathogen-inducible promoter, a chemical-regulated promoter, a tissue-preferred promoter, and any combinations thereof. In some embodiments one or more construct further comprises an ancillary sequence. In some embodiments the ancillary sequence is selected from the group consisting of a linker, an adapter, an intron, a restriction site, an insulator, a selectable marker, and any combinations thereof. In some embodiments the plant cell is from a monocot. In some embodiments the monocot is selected from the group consisting of maize, rice, sorghum, wheat, sugarcane and millet. In some embodiments the plant cell is from a dicot. In some embodiments the dicot is selected from the group consisting of canola, Brassica, soybean, sunflower, and cotton. In some embodiments the recombinase-mediated recombination results in about 2 or fewer integration events.

### DETAILED DESCRIPTION

The present disclosure is described more fully hereinafter, in which some, but not all embodiments of the disclosure are explicitly shown. These disclosures may be embodied in many different forms and should not be construed as limited to the embodiments set forth herein; rather, these embodiments are provided so that this disclosure will satisfy applicable legal requirements. Many modifications and other embodiments of the disclosures set forth herein will come to mind to one skilled in the art to which this disclosure pertains having the benefit of the teachings presented herein. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

Various methods and compositions for site-specific recombination, such as targeting the insertion of a polynucleotide of interest into a specific chromosomal site in the genome of a plant cell using a bacterium-mediated transformation method are provided. In some embodiments, a cell proliferation factor such as a babyboom polypeptide and/or a WUS/WOX homeobox polypeptide, are provided. According to the presently disclosed methods, at least one single-stranded T-DNA comprising a transfer cassette is provided to a plant cell to target the modification of a genomic target locus in a plant cell. In some embodiments, the target locus modification comprises insertion of a polynucleotide of interest at the target locus of the plant cell.

In some embodiments, a bacterium-mediated transformation method is used to provide a single-stranded T-DNA comprising a transfer cassette to a plant cell comprising a target locus having two non-identical recombination sites. The transfer cassette is flanked by a right border and a left border, and between the right border and the left border comprises in operable linkage and no particular order: a polynucleotide construct comprising a polynucleotide of interest, the polynucleotide construct flanked by a first recombination site and a second non-identical recombination site; and, a recombinase construct encoding a recombinase, wherein the plant cell comprises a genomic target locus comprising the first recombination site and the second non-identical recombination site, whereby the recombinase recognizes and implements recombination at the first recombination site and the second non-identical recombination site such that the polynucleotide construct is inserted at the target locus. In some embodiments, recombinase-mediated insertion results in about 5 or fewer integration events in the plant cell genome. In some embodiments, recombinase-mediated insertion results in a single copy of the polynucleotide construct integrated at the target locus. In some embodiments, the recombinase construct is flanked by a third recombination site and a fourth recombination site, wherein the third recombination site and the fourth recombination site are recombinogenic with each other. In some embodiments the third recombination site and the fourth recombination site are identical recombination sites. In some embodiments, the transfer cassette further comprises a cell proliferation factor construct encoding a cell proliferation factor. In some embodiments, the cell proliferation factor construct is flanked by a third recombination site and a fourth recombination site, wherein the third recombination site and the fourth recombination site are recombinogenic with each other. In some embodiments the third recombination site and the fourth recombination site are identical recombination sites. In some embodiments, a transfer cassette comprises a recombinase construct and a cell proliferation factor construct, wherein the recombinase construct and the cell proliferation factor construct are flanked by a third recombination site and a fourth recombination site, wherein the third recombination site and the fourth recombination site are recombinogenic with each other. In some embodiments the third recombination site and the fourth recombination site are identical recombination sites. In some embodiments, a transfer cassette further comprises a second recombinase construct encoding a second recombinase, wherein the second recombinase is different from the first recombinase. In some embodiments, a first recombinase construct, a cell proliferation factor construct, a second recombinase construct, or any combination thereof are flanked by a third recombination site and a fourth recombination site, wherein the third recombination site and the fourth recombination site are recombinogenic with each other, and a second recombinase recognizes and implements recombination at the third recombination site and the fourth recombination site. In some embodiments the third recombination site and the fourth recombination site are identical recombination sites. In some embodiments, a cell proliferation factor construct, a first recombinase construct, a second recombinase construct if present, and any combination thereof are flanked by a third recombination site and a fourth recombination site, and located within a first recombination site and a second non-identical recombination site.

In some embodiments, a transfer cassette is flanked by a right border and a left border, and between the right border and the left border comprises in operable linkage and no particular order: a polynucleotide construct comprising a polynucleotide of interest, the polynucleotide construct flanked by a first recombination site, a second non-identical recombination site and a third non-identical recombination site; a cell proliferation factor construct encoding a cell proliferation factor and flanked by the third recombination site and a fourth recombination site, wherein the fourth recombination site is identical to the third recombination site and located within the first recombination site and the second non-identical recombination site; a first recombinase construct encoding a first recombinase, and positioned within the third recombination site and the fourth identical recombination site; and a second recombinase construct encoding a second recombinase, and located within the third recombination site and the fourth identical recombination site; wherein the plant cell comprises a genomic target locus comprising the first recombination site and the second non-identical recombination site, whereby the first recombinase recognizes and implements recombination at the first recombination site and the second non-identical recombination site such that the polynucleotide construct and the third recombination site is inserted at the target locus, and the second recombinase recognizes and implements recombination at the third non-identical recombination site and the fourth identical recombination site thereby excising any construct located between the third non-identical recombination site and the fourth identical recombination site. In some embodiments, recombinase-mediated insertion results in about 5 or fewer integration events in the plant cell genome. In some embodiments, recombinase-mediated insertion results in a single copy of the polynucleotide construct integrated at the target locus.

In some embodiments, a transfer cassette is flanked by a right border and a left border and comprises between the right and left borders, in operable linkage and no particular order: a first recombination site within the right border and a second non-identical recombination site within the left border; a polynucleotide construct comprising the polynucleotide of interest, the polynucleotide construct flanked by a third recombination site and a fourth non-identical recombination site, wherein the third recombination site and the fourth non-identical recombination site are different from the first recombination site and the second non-identical recombination site; a cell proliferation factor construct encoding a cell proliferation factor, wherein the cell proliferation factor construct is flanked by the first recombination site or the second non-identical recombination site and a fifth recombination site, wherein the fifth recombination site identical to the other (i.e., the first recombination site or the second non-identical) recombination site flanking the cell proliferation factor construct; a first recombinase construct encoding a first recombinase, the first recombinase construct flanked by the third recombination site and the fifth recombination site, wherein the third recombination site and fifth recombination site are non-identical; and a second recombinase construct encoding a second recombinase, wherein the second recombinase is different from the first recombinase, wherein the second recombinase construct is flanked by the fourth recombination site and a sixth recombination site, wherein the sixth recombination site is identical to the fourth recombination site; wherein the plant cell comprises a genomic target locus comprising the second non-identical recombination site and the third non-identical recombination site; whereby the second recombinase recognizes and implements recombination at the second non-identical recombination site, the third non-identical recombination site, the fourth recombination site, and the sixth recombination site such that the polynucleotide construct is inserted at the target locus and the target locus further comprises the recombination product of the fourth recombination site and the sixth identical recombination site; and wherein the first recombinase recognizes and implements recombination at the first recombination site and the fifth recombination site thereby excising any construct located between the first recombination site and the fifth recombination site. In some embodiments, recombinase-mediated insertion results in about 5 or fewer integration events in the plant cell genome. In some embodiments, recombinase-mediated insertion results in a single copy of the polynucleotide construct integrated at the target locus.

In some embodiments, a transfer cassette is flanked by a right border and a left border and comprises between the right and left borders, in operable linkage and no particular order: a first recombination site within the right border and a second non-identical recombination site within the left border; a polynucleotide construct comprising the polynucleotide of interest, the polynucleotide construct flanked by a third recombination site and a fourth non-identical recombination site, wherein the third recombination site is the same as or different from the first recombination site and different from the second non-identical recombination site, the fourth recombination site different from the first recombination site, the second non-identical recombination site, the third recombination site, a fifth recombination site and a sixth recombination site; a cell proliferation factor construct comprising in no particular order a cell proliferation factor and a first recombinase construct, wherein the cell proliferation construct is flanked by the first recombination site or the second non-identical recombination site and the sixth recombination site, and wherein the sixth recombination site is identical to the other recombination site flanking the cell proliferation factor construct (i.e., the first recombination site or the second non-identical recombination site); and, the second recombinase construct encoding a second recombinase which is different from the first recombinase, wherein the second recombinase construct is flanked by the third non-identical recombination site and the fifth recombination site, wherein the fifth recombination site is the same as or different from the first recombination site, the third recombination site and the sixth recombination site, and is different from the second non-identical recombination site and the fourth recombination site; wherein the plant cell comprises a genomic target locus comprising the second non-identical recombination site and the third non-identical recombination site; whereby the second recombinase recognizes and implements recombination at the second non-identical recombination site, the third non-identical recombination site, the fourth recombination site, and the fifth recombination site such that the polynucleotide construct is inserted at the target locus and the target locus further comprises the recombination product of the fourth recombination site and the fifth identical recombination site; and wherein the first recombinase recognizes and implements recombination at the first recombination site and the sixth recombination site thereby excising any construct located between the first recombination site and the sixth recombination site. In some embodiments, recombinase mediated insertion results in about 5 or fewer integration events in the plant cell genome. In some embodiments, recombinase-mediated insertion results in a single copy of the polynucleotide construct integrated at the target locus.

In some embodiments, a bacterium-mediated transformation method is used to provide a first and a second single-stranded T-DNA to a plant cell comprising a target locus having two non-identical recombination sites, where the first T-DNA comprises a first transfer cassette and the second T-DNA comprises a second transfer cassette. In some embodiments, the plant cell is provided wherein a first transfer cassette is flanked by a right border and a left border, and between the right and the left borders comprises in operable linkage a polynucleotide construct comprising the polynucleotide of interest flanked by a first recombination site and a second non-identical recombination site; and a second transfer cassette comprising a recombinase construct encoding a recombinase; wherein the plant cell comprises a genomic target locus comprising the first recombination site and the second non-identical recombination site, whereby the recombinase recognizes and implements recombination at the first recombination site and the second non-identical recombination site such that the polynucleotide construct is inserted at the target locus. In some embodiments, recombinase-mediated insertion results in about 5 or fewer integration events in the plant cell genome. In some embodiments, recombinase-mediated insertion results in a single copy of the polynucleotide construct integrated at the target locus. In some embodiments, a first or a second transfer cassette further comprises a cell proliferation factor construct encoding a cell proliferation factor. In some embodiments a recombinase construct and a cell proliferation factor construct are on the same transfer cassette. In other embodiments a recombinase construct and a cell proliferation factor are on different transfer cassettes. In some embodiments a cell proliferation factor construct, a recombinase construct, or both constructs are flanked by a third recombination site and a fourth recombination site, wherein the third recombination site and the fourth recombination site are recombinogenic with each other, and each non-identical to a first recombination site and a second non-identical recombination site. In some embodiments, the third recombination site and the fourth recombination site are identical recombination sites. In some embodiments, a first or a second transfer cassette further comprises a second recombinase construct encoding a second recombinase, wherein the second recombinase is different from the first recombinase. In some embodiments, a second recombinase construct is on a different transfer cassette than a first recombinase construct. In some embodiments, a first transfer cassette comprises a polynucleotide construct comprising a polynucleotide of interest and flanked by a first recombination site and a second non-identical recombination site, and a first recombinase construct encoding a first recombinase; and a second transfer cassette comprises a cell proliferation factor construct encoding a cell proliferation factor, the cell proliferation factor construct flanked by a third recombination site and a fourth identical recombination site, wherein the third recombination site and the fourth identical recombination site are different from the first recombination site and the second non-identical recombination site, and a second recombinase construct encoding a second recombinase, wherein the first recombinase recognizes and implements recombination at the first recombination site and the second non-identical recombination site such that the polynucleotide construct is inserted at the target locus, and wherein the second recombinase recognizes and implements recombination at the third recombination site and the fourth recombination site thereby excising any construct located between the third and the fourth identical recombination site. In some embodiments, recombinase-mediated insertion results in about 5 or fewer integration events in the plant cell genome. In some embodiments, recombinase-mediated insertion results in a single copy of the polynucleotide construct integrated at the target locus. In some embodiments a second transfer cassette comprises a cell proliferation factor construct encoding a cell proliferation factor, and a second recombinase construct encoding a second recombinase wherein the cell proliferation factor construct and the second recombinase construct are flanked by a third recombination site and a fourth identical recombination site, wherein the third recombination site and the fourth identical recombination site are different from a first recombination site and a second non-identical recombination site. In some embodiments a second transfer cassette comprises a cell proliferation factor construct encoding a cell proliferation factor, a first recombinase construct encoding a first recombinase, and a second recombinase construct encoding a second recombinase wherein the cell proliferation factor construct and the first recombinase construct are flanked by a third recombination site and a fourth identical recombination site, wherein the third recombination site and the fourth identical recombination site are different from a first recombination site and a second non-identical recombination site. In some embodiments a second transfer cassette comprises aq cell proliferation factor construct encoding a cell proliferation factor, a first recombinase construct encoding a first recombinase, and a second recombinase construct encoding a second recombinase wherein the cell proliferation factor construct, the first recombinase construct, and the second recombinase construct are flanked by a third recombination site and a fourth identical recombination site, wherein the third recombination site and the fourth identical recombination site are different from a first recombination site and a second non-identical recombination site.

In some embodiments, a first transfer cassette comprises a polynucleotide construct comprising a polynucleotide of interest and flanked by a first recombination site and a second non-identical recombination site, and further comprises a first recombinase construct encoding a first recombinase, wherein the first recombinase construct is flanked by a third recombination site and a fourth recombination site, where the third recombination site is non-identical to the first recombination site and the second non-identical recombination site, and the fourth recombination site is recombinogenic or identical to the third recombination site; a second transfer cassette comprises a cell proliferation factor construct encoding a cell proliferation factor, wherein the cell proliferation factor construct is flanked by a fifth recombination site and a sixth recombination site, wherein the fifth recombination site and the sixth recombination site are non-identical to the first recombination site and the second non-identical recombination site, and are recombinogenic or identical to each other, and are optionally recombinogenic or identical to the third recombination site and/or the fourth recombination site, wherein the first recombinase recognizes and implements recombination at the first recombination site and the second non-identical recombination site such that the polynucleotide construct and the third non-identical recombination site are inserted at the target locus, and wherein the first recombinase recognizes and implements recombination at the third recombination site and the fourth recombination site and the fifth recombination site and the sixth recombination site thereby excising any construct located between the third recombination site and the fourth recombination site and/or between the fifth recombination site and the sixth recombination site. In some embodiments, recombinase-mediated reactions result in about 5 or fewer integration events in the plant cell genome. In some embodiments, recombinase-mediated reactions result in a single copy of the polynucleotide construct integrated at the target locus.

In some embodiments, a first transfer cassette comprises a polynucleotide construct comprising a polynucleotide of interest and a first recombinase construct encoding a first recombinase, wherein the polynucleotide construct and the first recombinase construct are flanked by a first recombination site and a second non-identical recombination site; and a second transfer cassette comprises a cell proliferation factor construct encoding a cell proliferation factor and a second recombinase construct encoding a second recombinase, where the cell proliferation factor construct and the second recombinase construct are flanked by a third recombination site and a fourth recombination site, wherein the third recombination site and the fourth recombination site are non-identical to the first recombination site and the second non-identical recombination site, and the third recombination site and the fourth recombination site are recombinogenic or identical to each other; wherein the first recombinase recognizes and implements recombination at the first recombination site and the second non-identical recombination site such that the polynucleotide construct is inserted at the target locus, and wherein the second recombinase recognizes and implements recombination at the third recombination site and the fourth recombination site thereby excising any construct located between the third recombination site and the fourth recombination site. In some embodiments, recombinase mediated reactions result in about 5 or fewer integration events in the plant cell genome. In some embodiments, recombinase-mediated reactions result in a single copy of the polynucleotide construct integrated at the target locus. In other embodiments, a second recombinase construct is provided on a first transfer cassette, and a first recombinase construct is provided on a second transfer cassette. In some embodiments, a first transfer cassette comprising a first recombinase construct or a second recombinase construct further comprises a third recombination site and a fourth recombinogenic or identical recombination site, wherein the recombinase construct is flanked by the third recombination site and the fourth recombination site and is within a first recombination site and a second non-identical recombination site flanking a polynucleotide construct.

Various transfer cassettes, transfer cassette elements, transfer cassette configurations, target loci, and recombination products are illustrated herein, and many modifications and other embodiments are expressly taught or disclosed to a person of skill in the art. Various T-DNAs, transfer cassettes, constructs comprising operably linked construct elements, and target locus recombination products are summarized in Table 1 below, wherein gDNA is genomic DNA, RS# is recombination site, where different numbers indicate non-identical sites and the same number indicates recombinogenic or identical sites, POI is the polynucleotide construct comprising a promoterless marker and/or an expression cassette comprising a promoter, a gene of interest, and a terminator, R# is the recombinase construct, where different numbers indicate different recombinases, R2 and its recombination sites are shown in italicized font, RB is the T-DNA right border, LB is the T-DNA left border, CPF is the cell proliferation factor construct, and Target Locus* is the modified target locus after the recombinase-mediated reaction(s). Transfer cassette elements are in no particular order except as dictated by the intended recombination reaction and product, for example, in transfer cassette B, the CPF construct and the recombinase construct (R1) could be positioned on either side of the POI construct flanked by non-identical recombination sites and still yield the same modified target locus recombination product. Unless otherwise stated, the constructs comprise any regulatory elements needed for expression in a plant, plant cell, and/or plant tissue.

TABLE 1
Target Locus: gDNA-Pro-RS1---RS2-gDNA

| | | |
|---|---|---|
| A | Transfer Cassette | RB-RS1-POI-RS2-R1-LB |
| | Target Locus* | gDNA-Pro-RS1-POI-RS2-gDNA |
| B | Transfer Cassette | RB-R1-RS1-POI-RS2-CPF-LB |
| | Target Locus* | gDNA-Pro-RS1-POI-RS2-gDNA |
| C | Transfer Cassette | RB-RS1-POI-RS2-RS3-R1-RS3-LB |
| | Target Locus* | gDNA-Pro-RS1-POI-RS2-gDNA |
| D | Transfer Cassette | RB-R1-RS1-POI-RS2-RS3-CPF-RS3-LB |
| | Target Locus* | gDNA-Pro-RS1-POI-RS2-gDNA |
| E | Transfer Cassette | RB-RS3-CPF-RS3-RS1-POI-RS2-R1-LB |
| | Target Locus* | gDNA-Pro-RS1-POI-RS2-gDNA |
| F | Transfer Cassette | RB-R1-RS1-POI-RS2-RS3-R2-CPF-RS3-LB |
| | Target Locus* | gDNA-Pro-RS1-POI-RS2-gDNA |
| F1 | Transfer Cassette | RB-RS3-R2-CPF-RS3-RS1-POI-RS2-R1-LB |
| | Target Locus* | gDNA-Pro-RS1-POI-RS2-gDNA |
| G | Transfer Cassette | RB-RS3-CPF-RS3-R2-RS1-POI-RS2-R1-LB |
| | Target Locus* | gDNA-Pro-RS1-POI-RS2-gDNA |
| H | Transfer Cassette | RB-RS1-POI-RS3-CPF-R1-RS3-RS2-LB |
| | Target Locus* | gDNA-Pro-RS1-POI-RS3-RS2-gDNA |
| I | Transfer Cassette | RB-RS1-RS3-CPF-R2-R1-RS3-POI-RS2-LB |
| | Target Locus* | gDNA-Pro-RS1-RS3-POI-RS2-gDNA |
| J | Transfer Cassette | RB-RS4-CPF-RS4-R2-RS1-POI-RS3-R1-RS3-RS2-LB |
| | Target Locus* | gDNA-Pro-RS1-POI-RS3-RS2-gDNA |
| K | Transfer Cassette | RB-RS4-CPF-R2-RS4-RS1-POI-RS3-R1-RS3-RS2-LB |
| | Target Locus* | gDNA-Pro-RS1-POI-RS3-RS2-gDNA |
| L | Transfer Cassette | RB-RS3-CPF-R2-RS3-RS1-POI-RS3-R1-RS3-RS2-LB |
| | Target Locus* | gDNA-Pro-RS1-POI-RS3-RS2-gDNA |
| M | Transfer Cassette1 | Rb-RS1-POI-RS2-LB |
| | Transfer Cassette2 | RB-R1-LB |
| | Target Locus* | gDNA-Pro-RS1-POI-RS2-gDNA |
| N | Transfer Cassette1 | RB-RS1-POI-RS2-LB |
| | Transfer Cassette2 | RB-CPF-R1-LB |
| | Target Locus* | gDNA-Pro-RS1-POI-RS2-gDNA |
| O | Transfer Cassette1 | RB-RS1-POI-RS2-R1-LB |
| | Transfer Cassette2 | RB-CPF-LB |
| | Target Locus* | gDNA-Pro-RS1-POI-RS2-gDNA |
| P | Transfer Cassette1 | RB-RS1-POI-RS2-LB |
| | Transfer Cassette2 | RB-RS3-CPF-RS3-R1-LB |
| | Target Locus* | gDNA-Pro-RS1-POI-RS2-gDNA |
| Q | Transfer Cassette1 | RB-RS1-POI-RS2-LB |
| | Transfer Cassette2 | RB-RS3-CPF-R1-RS3-LB |
| | Target Locus* | gDNA-Pro-RS1-POI-RS2-gDNA |
| R | Transfer Cassette1 | RB-RS1-POI-RS2-R1-LB |
| | Transfer Cassette2 | RB-RS3-CPF-RS3-R2-LB |
| | Target Locus* | gDNA-Pro-RS1-POI-RS2-gDNA |
| S | Transfer Cassette1 | RB-RS1-POI-RS2-R1-LB |
| | Transfer Cassette2 | RB-RS3-R2-CPF-RS3-LB |
| | Target Locus* | gDNA-Pro-RS1-POI-RS2-gDNA |
| T | Transfer Cassette1 | RB-RS1-POI-RS2-LB |
| | Transfer Cassette2 | RB-RS3-CBF-R1-RS3-R2-LB |
| | Target Locus* | gDNA-Pro-RS1-POI-RS2-gDNA |
| U | Transfer Cassette1 | RB-RS1-POI-RS2-LB |
| | Transfer Cassette2 | RB-RS3-CBF-R2-RS3-R1-LB |
| | Target Locus* | gDNA-Pro-RS1-POI-RS2-gDNA |
| V | Transfer Cassette1 | RB-RS1-POI-RS2-LB |
| | Transfer Cassette2 | RB-RS3-CBF-R2-R1-RS3-LB |
| | Target Locus* | gDNA-Pro-RS1-POI-RS2-gDNA |
| W | Transfer Cassette1 | RB-RS1-POI-RS3-R1-RS3-RS2-LB |
| | Transfer Cassette2 | RB-RS3-CPF-RS3-LB |
| | Target Locus* | gDNA-Pro-RS1-POI-RS3-RS2-gDNA |
| X | Transfer Cassette1 | RB-RS1-POI-R1-RS2-LB |
| | Transfer Cassette2 | RB-RS3-CPF-R2-RS3-LB |
| | Target Locus* | gDNA-Pro-RS1-POI-R1-RS2-gDNA |
| Y | Transfer Cassette1 | RB-RS1-POI-RS3-R1-RS3-RS2-LB |
| | Transfer Cassette2 | RB-RS3-CPF-R2-RS3-LB |
| | Target Locus* | gDNA-Pro-RS1-POI-RS3-RS2-gDNA |
| Z | Transfer Cassette1 | RB-RS1-POI-RS2-LB |
| | Transfer Cassette2 | RB-CPF-LB |
| | Transfer Cassette3 | RB-R1-LB |
| | Target Locus* | gDNA-Pro-RS1-POI-RS2-LB |
| AA | Transfer Cassette1 | RB-RS1-POI-RS2-LB |
| | Transfer Cassette2 | RB-RS3-CPF-RS3-LB |
| | Transfer Cassette3 | RB-R1-LB |
| | Target Locus* | gDNA-Pro-RS1-POI-RS2-LB |
| BB | Transfer Cassette1 | RB-RS1-POI-RS2-LB |
| | Transfer Cassette2 | RB-RS3-CPF-RS3-R2-LB |
| | Transfer Cassette3 | RB-R1-LB |
| | Target Locus* | gDNA-Pro-RS1-POI-RS2-LB |

The target locus comprises a first recombination site and a second recombination site, wherein the first recombination site and the second recombination site are non-identical to each other. The target locus comprises a promoter trap located outside of a first recombination site and a second non-identical recombination site, the promoter trap being a promoter active in a plant. For example, the target locus is similar to or derived from the target locus illustrated in Table 1 above as gDNA-Pro-RS1-RS2-gDNA. The target locus is designed such that a recombinase-mediated recombination reaction will create an operable linkage of the promoter to the promoterless polynucleotide construct. This has several uses, including but not limited to facilitating identification of recombination events.

In some embodiments, a bacterium-mediated transformation results in about 5 or fewer integration events in the plant cell genome. In some embodiments, a bacterium-mediated transformation results in about 5, 4, 3, 2, or 1 integration events in the plant cell genome. In some embodiments, a bacterium-mediated transformation results in about 5, 4, 3, 2, or 1 integration events at the target locus in the plant cell genome. In some embodiments, a bacterium-mediated transformation results in about 5, 4, 3, 2, or 1 integration events exclusively at the target locus in the plant cell genome. In some embodiments, recombinase mediated reactions result in about 5 or fewer integration events in the plant cell genome. In some embodiments, recombinase mediated reaction(s) results in about 5, 4, 3, 2, or 1 integration events in the plant cell genome. In some embodiments, recombinase mediated reaction(s) results in about 5, 4, 3, 2, or 1 integration events at the target locus in the plant cell genome. In some embodiments, recombinase mediated reaction(s) results in about 5, 4, 3, 2, or 1 integration events exclusively at the target locus in the plant cell genome. In some embodiments, the methods further comprise selecting for integration events wherein a cell proliferation factor, a first recombinase, a second recombinase, or any combination thereof is present in the plant cell genome. In other embodiments, the methods further comprise selecting for integration events wherein a cell proliferation factor, a first recombinase, a second recombinase, or any combination thereof is not present in the plant cell genome.

Bacterial strains capable of transferring DNA to plants are well-known, as are the various vector elements, vector constructs and vector construct strategies used in the methods. The most well-established system to generate transgenic plants is Agrobacterium-mediated genetic transformation. Initially this technology involved complex microbial genetic methodologies that inserted a gene of interest into the transfer DNA (T-DNA) region of large tumor-inducing plasmids (Ti-plasmids) or root-inducing plasmids (Ri-plasmids). Later it was discovered that T-DNA transfer could still be done if the T-DNA region and the virulence (vir) genes required for T-DNA processing and transfer were split into two replicons. This binary system allowed for easier manipulation of Agrobacterium. Similar components, constructs and the like are known in other bacterial systems. In some embodiments, one or more T-DNAs are on a single Ti or Ri plasmid. In other embodiments, one or more T-DNAs are on separate plasmids. In some embodiments a binary vector system or a superbinary vector system is used.

Site-specific recombination, also known as conservative site-specific recombination, is a type of genetic recombination in which DNA strand exchange takes place between segments possessing only a limited degree of sequence homology. Site-specific recombinases (SSRs) perform rearrangements of DNA segments by recognizing and binding to short DNA sequences (sites, recombinase sites, recognition sites), at which they cleave the DNA backbone, exchange the two DNA helices involved and rejoin the DNA strands. While in some site-specific recombination systems just a recombinase enzyme and the recombination sites is enough to perform all these reactions, in other systems a number of accessory proteins and/or accessory sites are also needed. Multiple genome modification strategies, among these recombinase-mediated cassette exchange (RMCE), an advanced approach for the targeted introduction of transcription units into predetermined genomic loci, rely on the capacities of SSRs. Recombination sites are typically between 30 and 200 nucleotides in length, but can be over 900 nucleotides long, and consist of two motifs with a partial inverted-repeat symmetry, to which the recombinase binds, and which flank a central crossover sequence at which the recombination takes place. Truncated sites, including minimal sites, which retain function are well-known in the art. For example a minimal FRT recombination site has been characterized and comprises a pair of 11-13 base pair symmetry elements (FLP binding sites), the 8 base pair core, or spacer, region, and, the polypyrimidine tracts. The lox sites of the Cre recombinase system have a similar minimal site structure. Any recombination site recognized by the appropriate recombinase can be used in the methods and compositions. In some embodiments, any one or more of the recombination sites in the target site and/or the transfer cassette may be a full length site. In other embodiments, any one or more of the recombination sites in the target site and/or the transfer cassette may be a truncated or minimal recombination site.

The compositions and methods can be used with any plant, plant tissue, or plant cell. In some embodiments, the plant, plant tissue, or plant cell is or is from a monocot or a dicot. In some embodiments, the plant includes but is not limited to corn (maize), Brassica spp. including canola, alfalfa , rice, rye, sorghum, millet including pearl millet, proso millet, foxtail millet, finger millet, sunflower, safflower, wheat, soybean, tobacco, Arabidopsis, potato, peanuts, cotton, sweet potato, cassava, coffee, coconut, palm, pineapple, Citrus spp., cocoa, tea, banana, avocado, fig, guava, mango, olive, papaya, cashew, macadamia, almond, sugar beets, sugarcane, oats, barley, beans, peas, vegetables, ornamentals, and conifers. In some embodiments, the monocot is maize, rice, sorghum, wheat, sugarcane, millet, or other monocot. In some embodiments the dicot is canola, Brassica spp., soybean, sunflower, cotton, or other dicot.

A target site or target locus is a polynucleotide comprising a nucleotide sequence comprising at least one recombination site recognized by a recombinase, in some embodiments the target site is found in the genome of a plant cell. Target sites include both native and non-native sites, wherein native refers to either sequence and/or in genomic location to the plant genome. In some embodiments, the target site comprises at least 1, 2, 3, 4, 5, 6 or more recombination sites for site-specific recombination. In some embodiments the target locus comprises two non-identical recombination sites. One or more intervening sequences may be present between the recombination sites of the target locus. Intervening sequences include but are not limited to native genomic sequence, linkers, adapters, selectable markers, polynucleotides of interest, other recombination sites, promoters and/or other sites that aid in vector construction or analysis. Various polynucleotides of interest then could be employed between the recombination sites. In addition, the recombination sites of the target site can be located in various positions, including, for example, within intron sequences, coding sequences, or untranslated regions.

The recombination sites employed in the methods and compositions can be recombinogenic or identical sites, or dissimilar or non-identical sites. Recombinogenic or identical sites have the same or similar sequence, and in the presence of the appropriate recombinase, will recombine with one another. Dissimilar or non-identical recombination sites have distinct sequences (i.e., have at least one nucleotide difference), and in the presence of the appropriate recombinase, will have minimal or no recombination with each other. Dissimilar or non-identical sites include those sites that recombine (or excise) with one another at a frequency lower than the detectable limit under standard conditions in an excision assay, including but not limited to lower than 5%, 4%, 3%, 2%, 1.5%, 1%, 0.75%, 0.5%, 0.25%, 0.1%, 0.075, 0.005%, 0.001% recombination (or excision). Each recombination site within the set of non-identical recombination sites is biologically active and therefore can recombine with an identical site.

A transfer cassette comprises at least a first recombination site, a polynucleotide construct, and a second recombination site, wherein the first recombination site and second recombination sites are non-identical and correspond to the recombination sites at the target locus. Any combination of recombination sites can be used in the transfer cassettes to provide a polynucleotide of interest, including recombination sites from one or more recombination systems.

A recombinase is a polypeptide that catalyzes site-specific recombination between compatible recombination sites (see, e.g., Sauer (1994) Curr Op Biotechnol 5:521-527; Sadowski (1993) FASEB 7:760-767; and Thomson & Ow (2006) Genesis 44:465-476, the contents of which are incorporated herein by reference. The recombinase used can be a naturally occurring recombinase or a biologically active fragment or variant of the recombinase. Any recombinase system or combination of recombinase systems can be used in the compositions and methods herein. In some embodiments, the first and/or the second recombinase is FLP, Cre, chimeric FLP-Cre, S-CRE, V-CRE, Dre, SSV1, lambda Int, phi C31 Int, HK022, R, Gin, Tn1721, CinH, ParA, Tn5053, Bxb1, TP907-1, U153 or a chimeric recombinase. Examples of site-specific recombination systems used in plants can be found in U.S. Pat. Nos. 5,929,301, 6,175,056, 6,331,661; and WO1999025821, WO1999025855, WO1999025841, and WO1999025840, the contents of each are herein incorporated by reference. An exemplary chimeric recombinase is chimeric FLP-Cre recombinase described in WO1999025840, herein incorporated by reference. Recombinase systems include but are not limited to the Cre-lox system from bacteriophage P1 (Baubonis & Sauer (1993) Nucl Acids Res 21:2025-2029; Albert et al. (1995) Plant J. 7:649-59), the FLP-FRT system from S. cerevisiae (O′Gorman et al. (1991) Science 251:1351-1355), the R-RS system of Zygosaccharomyces rouxii (Onouchi et al. (1995) Mol Gen Genet 247:653-660), the Gin-gix system, including a modified Gin-gix system from bacteriophage Mu (Maeser & Kahmann (1991) Mol Gen Genet 230:170-76), the β-recombinase-six system from a Bacillus subtilis plasmid (Diaz et al. (1999) J Biol Chem 274:6634-6640), and the γδ-res system from the bacterial transposon Tn1000 (Schwikardi & Dorge (2000) FEBS Lett 471:147-150). Cre, FLP, R, Gin, β-recombinase and γδ are the recombinases, and lox, FRT, RS, gix, six and res the respective recombination sites (reviewed by Sadowski (1993) FASEB J 7:750-67; Ow & Medberry (1995) Crit Rev Plant Sci 14:239-261).

In some embodiments, recombinases from the Integrase or Resolvase families are used, including biologically active variants and fragments thereof. The Integrase family of recombinases has over one hundred members and includes, for example, FLP, Cre, lambda integrase, and R. The Integrase family has been grouped into two classes based on the structure of the active sites, serine recombinases and tyrosine recombinases. The tyrosine family, which includes Cre, FLP, SSV1, and lambda integrase, uses the catalytic tyrosine's hydroxyl group for a nucleophilic attack on the phosphodiester bond of the DNA. Typically, members of the tyrosine family initially nick the DNA, which later forms a double strand break. In the serine recombinase family, which includes phiC31 integrase, a conserved serine residue forms a covalent link to the DNA target site (Grindley et al. (2006) Ann Rev Biochem 16:16). For other members of the Integrase family, see, for example, Esposito et al. (1997) Nucl Acids Res 25:3605-3614; and Abremski et al. (1992) Protein Eng 5:87-91; each of which is herein incorporated by reference in its entirety. Other recombination systems include, for example, the Streptomycete bacteriophage phi C31 (Kuhstoss et al. (1991) J Mol Biol 20:897-908); the SSV1 site-specific recombination system from Sulfolobus shibatae (Maskhelishvili et al. (1993) Mol Gen Genet 237:334-342); and a retroviral integrase-based integration system (Tanaka et al. (1998) Gene 17:67-76). In some embodiments, the recombinase does not require cofactors or a supercoiled substrate; embodiments include but are not limited to Cre, FLP, or active variants or fragments thereof.

The FLP recombinase catalyzes a conservative site-specific reaction between two FRT sites, and is involved in amplifying the copy number of the two-micron plasmid of S. cerevisiae during DNA replication. The FLP protein has been cloned and expressed (Cox (1993) PNAS 80:4223-4227). In some embodiments, a polynucleotide encoding the recombinase can be modified to comprise more plant-preferred codons (see, e.g., U.S. Pat. No. 5,929,301, which is herein incorporated by reference in its entirety). Additional functional variants and fragments of FLP are known (Buchholz et al. (1998) Nat Biotechnol 16:657-662; Hartung et al. (1998) J Biol Chem 273:22884-22891; Saxena et al. (1997) Biochim Biophys Acta 1340:187-204; Hartley et al. (1980) Nature 286:860-864; Voziyanov et al. (2002) Nucl Acids Res 30:1656-1663; Zhu & Sadowski (1995) J Biol Chem 270:23044-23054; and U.S. Pat. No. 7,238,854, each of which is herein incorporated by reference in its entirety).

The bacteriophage recombinase Cre catalyzes site-specific recombination between two lox sites (see, e.g., Guo et al. (1997) Nature 389:40-46; Abremski et al. (1984) J Biol Chem 259:1509-1514; Chen et al. (1996) Somat Cell Mol Genet 22:477-488; Shaikh et al. (1977) J Biol Chem 272:5695-5702; and, Buchholz et al. (1998) Nat Biotechnol 16:657-662, each of which is herein incorporated by reference in its entirety). Cre polynucleotide sequences may also be synthesized using plant-preferred codons (see, e.g., WO1999025840, which is herein incorporated by reference in its entirety). Variants of the Cre recombinase are known (e.g., U.S. Pat. No. 6,890,726; Rufer & Sauer (2002) Nucl Acids Res 30:2764-2772; Wierzbicki et al. (1987) J Mol Biol 195:785-794; Petyuk et al. (2004) J Biol Chem 279:37040-37048; Hartung & Kisters-Woike (1998) J Biol Chem 273:22884-22891; Santoro & Schultz (2002) PNAS 99:4185-4190; Koresawa et al. (2000) J Biochem (Tokyo) 127:367-372; and Vergunst et al. (2000) Science 290:979-982, each of which is herein incorporated by reference in its entirety).

In some embodiments, a chimeric recombinase is used. A chimeric recombinase is a recombinant fusion protein which is capable of catalyzing site-specific recombination between recombination sites that originate from different recombination systems. For example, if the set of recombination sites comprises a FRT site and a LoxP site, a chimeric FLP/Cre recombinase or active variant or fragment thereof can be used, or both recombinases may be separately provided. Methods for the production and use of such chimeric recombinases or active variants or fragments thereof are described, for example, in WO1999025840; and Shaikh & Sadowski (2000) J Mol Biol 302:27-48, each of which is herein incorporated by reference in its entirety.

In other embodiments, a variant recombinase is used. Methods for modifying the kinetics, cofactor interaction and requirements, expression, optimal conditions, and/or recognition site specificity, and screening for activity of recombinases and variants are known, see for example Miller et al. (1980) Cell 20:721-9; Lange-Gustafson & Nash (1984) J Biol Chem 259:12724-32; Christ et al. (1998) J Mol Biol 288:825-36; Lorbach et al. (2000) J Mol Biol 296:1175-81; Vergunst et al. (2000) Science 290:979-82; Dorgai et al. (1995) J Mol Biol 252:178-88; Dorgai et al. (1998) J Mol Biol 277:1059-70; Yagu et al. (1995) J Mol Biol 252:163-7; Sclimente et al. (2001) Nucl Acids Res 29:5044-51; Santoro & Schultze (2002) PNAS 99:4185-90; Buchholz & Stewart (2001) Nat Biotechnol 19:1047-52; Voziyanov et al. (2002) Nucl Acids Res 30:1656-63; Voziyanov et al. (2003) J Mol Biol 326:65-76; Klippel et al. (1988) EMBO J 7:3983-9; Arnold et al. (1999) EMBO J 18:1407-14; and WO 2003008045, WO1999025840, and WO1999025841; each of which is herein incorporated by reference in its entirety. Assays for recombinase activity are known and generally measure the overall activity of the enzyme on DNA substrates containing recombination sites. For example, to assay for FLP activity, inversion of a DNA sequence in a circular plasmid containing two inverted FRT sites can be detected as a change in position of restriction enzyme sites (see, e.g., Vetter et al. (1983) PNAS 80:7284). Alternatively, excision of DNA from a linear molecule or intermolecular recombination frequency induced by the enzyme may be assayed, as described, for example, in Babineau et al. (1985) J Biol Chem 260:12313; Meyer-Leon et al. (1987) Nucl Acid Res 15:6469; and Gronostajski et al. (1985) J Biol Chem 260:12328. Alternatively, recombinase activity may also be assayed by excision of a sequence flanked by recombinogenic sites that upon removal will activate an assayable marker gene.

In some embodiments, a first recombination site, a second non-identical recombination site, a third recombination site, a fourth recombination site, a fifth recombination site, a sixth recombination site or an additional recombination site are FRT, lox, RS, gix, rox, integrase, invertase, resolvase, chimeric sites or any combinations thereof. In some embodiments the recombination sites are FRT sites, including but not limited to FRT1, FRT5, FRT6, FRT7, FRT12, FRT87 or any other FRT site including but not limited to those disclosed in WO2001023545, US20060195937, and WO2007011733, each of which is herein incorporated by reference in its entirety. In some embodiments the recombination sites are loxP, lox511, lox5171, lox2272, or any other lox sites including but not limited to those disclosed in US20070087366 which is herein incorporated by reference in its entirety.

Recombination sites from the Cre/Lox site-specific recombination system can also be used, including wild type LOX sites and mutant LOX sites. An analysis of the recombination activity of mutant LOX sites is presented in Lee et al. (1998) Gene 216:55-65, herein incorporated by reference in its entirety. Also, see for example, Schlake & Bode (1994) Biochemistry 33:12746-12751; Huang et al. (1991) Nucl Acids Res 19:443-448; Sadowski (1995) In Progress in Nucleic Acid Research and Molecular Biology Vol. 51, pp. 53-91; Cox (1989) In Mobile DNA, Berg and Howe (eds) American Society of Microbiology, Washington D.C., pp. 116-670; Dixon et al. (1995) Mol Microbiol 18:449-458; Umlauf & Cox (1988) EMBO J 7:1845-1852; Buchholz et al. (1996) Nucl Acids Res 24:3118-3119; Kilby et al. (1993) Trends Genet 9:413-421; Rossant & Geagy (1995) Nat Med 1: 592-594; Albert et al. (1995) Plant J 7:649-659; Bayley et al. (1992) Plant Mol Biol 18:353-361; Odell et al. (1990) Mol Gen Genet 223:369-378; Dale & Ow (1991) PNAS 88:10558-10562; Qui et al. (1994) PNAS 91:1706-1710; Stuurman et al. (1996) Plant Mol. Biol 32:901-913; Dale et al. (1990) Gene 91:79-85; and WO2001000158; each of which is herein incorporated by reference in its entirety.

Any recombination site may be a naturally occurring site or a site generated by random or directed sequence modification. Assays to measure the biological activity of recombination sites are known in the art. See, for example, Senecoll et al. (1988) J Mol Biol 201:406-421; Voziyanov et al. (2002) Nucl Acid Res 30:7; U.S. Pat. No. 6,187,994, WO2001000158, and Albert et al. (1995) Plant J 7:649-659. Methods to determine if a modified recombination site is recombinogenic are known (see, e.g., WO2007011733, which is herein incorporated by reference in its entirety). Variant recombinase recognition sites are known, see for example, Hoess et al. (1986) Nucl Acids Res 14:2287-300; Thomson et al. (2003) Genesis 36:162-7; Huang et al. (1991) Nucl Acids Res 19:443-8; Siebler & Bode (1997) Biochemistry 36:1740-7; Thygarajan et al. (2001) Mol Cell Biol 21:3926-34; Umlauf & Cox (1988) EMBO J 7:1845-52; Lee & Saito (1998) Gene 216:55-65; WO2001023545, WO1999025851, WO2001007572; and U.S. Pat. No. 5,888,732; each of which is herein incorporated by reference in its entirety.

The presently disclosed methods and compositions may utilize cell proliferation factors. While not being limited to any theory or mechanism, the cell proliferation factor(s) may enhance cell growth, stimulate embryogenesis, stimulate organogenesis, stimulate meristematic cell growth, transformation frequency, transformation event recovery, rates of targeted polynucleotide modification, transformation event quality, recombinase-mediated reaction frequency or quality, or any combination thereof. A cell proliferation factor is a polypeptide or a polynucleotide capable of stimulating growth of a cell or tissue, including but not limited to promoting progression through the cell cycle, inhibiting cell death, such as apoptosis, stimulating cell division, and/or stimulating embryogenesis. The polynucleotides can fall into several categories, including but not limited to, cell cycle stimulatory polynucleotides, developmental polynucleotides, anti-apoptosis polynucleotides, hormone polynucleotides, or silencing constructs targeted against cell cycle repressors or pro-apoptotic factors. Examples of each include but are not limited to: 1) cell cycle stimulatory polynucleotides including plant viral replicase genes such as RepA, cyclins, E2F, prolifera, cdc2 and cdc25; 2) developmental polynucleotides such as Lec1, Kn1 family, WUS/WOX homeobox, Zwille, BBM (babyboom (ODP2/BBM)), Aintegumenta (ANT), FUS3, and members of the Knotted family, such as Kn1, STM, OSH1, and SbH1; 3) anti-apoptosis polynucleotides such as CED9, Bc12, Bcl-X(L), Bcl-W, A1, McL-1, Macl, Boo, and Bax-inhibitors; 4) hormone polynucleotides such as IPT, TZS, and CKI-1; and 5) silencing constructs targeted against cell cycle repressors, such as Rb, CK1, prohibitin, and weel, or stimulators of apoptosis such as APAF-1, bad, bax, CED-4, and caspase-3, and repressors of plant developmental transitions, such as Pickle and WD polycomb genes including FIE and Medea. The polynucleotides can be silenced by any known method such as antisense, RNA interference (RNAi), topical RNAi, cosuppression, chimeraplasty, CRISPR/Cas, or transposon insertion.

The WUS/WOX homeobox polypeptide is a WUS1, WUS2, WUS3, WOX2A, WOX4, WOX5, or WOX9 polypeptide (van der Graaff et al., 2009, Genome Biology 10:248). The WUS/WOX homeobox polypeptide can be a monocot WUS/WOX homeobox polypeptide. In various embodiments, the WUS/WOX homeobox polypeptide can be a barley, maize, millet, oats, rice, rye, Setaria sp., sorghum, sugarcane, switchgrass, triticale, turfgrass, or wheat WUS/WOX homeobox polypeptide. Alternatively, the WUS/WOX homeobox polypeptide can be a dicot WUS/WOX homeobox polypeptide. In various embodiments, the WUS/WOX homeobox polypeptide can be a cotton, soybean, canola, sunflower, safflower, jatropha, castor, palm, cowpea, or flax WUS/WOX homeobox polypeptide.

In some embodiments, the cell proliferation factor is a member of the AP2/ERF family of proteins. The AP2/ERF family of proteins is a plant-specific class of putative transcription factors that regulate a wide variety of developmental processes and are characterized by the presence of an AP2 DNA binding domain that is predicted to form an amphipathic alpha helix that binds DNA (PFAM Accession PF00847). The AP2 domain was first identified in APETALA2, an Arabidopsis protein that regulates meristem identity, floral organ specification, seed coat development, and floral homeotic gene expression. The AP2/ERF proteins have been subdivided into distinct subfamilies based on the presence of conserved domains. Initially, the family was divided into two subfamilies based on the number of DNA binding domains, with the ERF subfamily having one DNA binding domain, and the AP2 subfamily having 2 DNA binding domains. As more sequences were identified, the family was subsequently subdivided into five subfamilies: AP2, DREB, ERF, RAV, and others (Sakuma et al. (2002) Biochem Biophys Res Comm 290:998-1009).

In some embodiments, the cell proliferation factor is a member of the AP2/ERF family of proteins. Members of the APETALA2 (AP2) family of proteins function in a variety of biological events, including but not limited to, development, plant regeneration, cell division, embryogenesis, and cell proliferation (see, e.g., Riechmann & Meyerowitz (1998) Biol Chem 379:633-646; Saleh & Pagés (2003) Genetika 35:37-50; and, Database of Arabidopsis Transcription Factors at daft.cbi.pku.edu.cn). The AP2 family includes, but is not limited to, AP2, ANT, Glossyl5, AtBBM, BnBBM, and maize ODP2/BBM (babyboom), as well as the sequences and motifs disclosed in US20110167516, herein incorporated by reference in its entirety.

In other embodiments, other cell proliferation factors, such as, Lec1, Kn1 family, WUSCHEL(WUS/WOX homeobox) (e.g., WUS1, WUS2, WUS3, WOX2A, WOX4, WOX5, or WOX9), Zwille, and Aintegumeta (ANT), may be used alone, or in combination with a babyboom polypeptide or other cell proliferation factor to enhance targeted polynucleotide modification in plants. See, for example, US2003/0135889, WO2003001902, and U.S. Pat. No. 6,512,165, each of which is herein incorporated by reference in its entirety. In some embodiments, the cell proliferation factor construct encodes a BBM (ODP2) polypeptide, a WUS/WOX homeobox polypeptide, or a combination thereof. When multiple cell proliferation factors are used, or when a babyboom polypeptide is used along with any of the abovementioned polypeptides, the polynucleotides encoding each of the factors can be present on the same transfer cassette or on separate transfer cassettes. Likewise, the polynucleotide(s) encoding the cell proliferation factor(s) and the polynucleotide encoding a recombinase or having the polynucleotide construct can be located on the same transfer cassette or on different transfer cassettes. When two or more factors are coded for by separate transfer cassettes, the transfer cassettes can be provided to the plant simultaneously or sequentially.

Polynucleotides can comprise deoxyribonucleotides, ribonucleotides and combinations of ribonucleotides and deoxyribonucleotides that include both naturally occurring molecules and synthetic analogues. The polynucleotides also encompass all forms of sequences including, but not limited to, single-, double-, or multi-stranded forms, hairpins, stem-and-loop structures, circular plasmids, and the like. The polynucleotide encoding a cell proliferation factor may be native to the cell or heterologous. A native polypeptide or polynucleotide comprises a naturally occurring amino acid sequence or nucleotide sequence. Heterologous in reference to a polypeptide or a nucleotide sequence is a polypeptide or a sequence that originates from a different species, or if from the same species, is substantially modified from its native form in composition and/or genomic locus by deliberate human intervention.

The methods provided herein comprise providing to a plant cell various polynucleotide constructs or polypeptides including, but not limited to transfer cassettes, site-specific recombinases, recombination sites, polynucleotides of interest or any active variants or fragments thereof provided herein. Further, the methods provide selecting at least one plant cell comprising integration of the polynucleotide construct at the target locus, such as selecting for cells expressing a screenable or selectable marker, which are known in the art.

As used herein, the term plant includes plant cells, plant protoplasts, plant cell tissue cultures from which a plant can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Progeny, variants, and mutants of the regenerated plants are also included herein, provided that these parts comprise the polynucleotide construct.

Providing includes any method or combination of methods that allows for a composition, such as a polynucleotide, to be brought together with the other recited components. A variety of methods are known in the art for the introduction of compositions into a plant, plant cell, or plant tissue. Methods for introducing compositions into plants, plant cells, or plant tissues are known in the art and include, but are not limited to, stable transformation methods, transient transformation methods, virus-mediated methods, and sexual breeding.

Various bacterium mediated transformation methods for targeting the insertion of a polynucleotide of interest at a target site for site specific recombination (SSI site) are described herein, wherein these methods may provide both a cell proliferation factor, such as but not limited to a developmental gene, (e.g., BBM and/or WUS; inducible or constitutive) with one or more recombinase (e.g., FLP and/or Cre; inducible or constitutive) along with a transfer cassette resulting in a markedly improved transformation method measured by the high frequency of SSI, and recovery of precise RMCE events. The cell proliferation factor can be co-delivered on a single T-DNA with the polynucleotide construct and/or the recombinase construct, or co-transformed as independent T-DNA molecules on different binary vectors. In some embodiments the bacterium may be an Agrobacterium or a Rhizobia, including but not limited to Rhizobium spp., Sinorhizobium spp., Mesorhizobium spp., Phyllobacterium spp. Ochrobactrum spp. including, but not limited to, Ochrobactrum haywardense H1, deposited under NRRL B-67078, Ochrobactrum cytisi, Ochrobactrum daejeonense, Ochrobactrum lupine, Ochrobactrum oryzae, Ochrobactrum tritici, LBNL124-A-10, HTG3-C-07 and Ochrobactrum pectoris, and Bradyrhizobium spp.

In some embodiments, a target site (target locus) can be introduced into the plant genome by any of the transformation methods known in the art. For example, the target site is provided as a polynucleotide construct and introduced into a plant or plant cell (e.g. as described in U.S. Pat. Nos. 6,187,994, 6,262,341, 6,330,545, and 6,331,661 and US20110047655, each of which is herein incorporated by reference in its entirety). Transformation protocols as well as protocols for introducing compositions into plants may vary depending on the type of plant or plant cell, i.e., monocot or dicot, targeted for transformation. Suitable methods of introducing compositions into plant cells, including polynucleotides and/or polypeptides, include microinjection (Crossway et al. (1986) Biotechniques 4:320-334), electroporation (Riggs et al. (1986) PNAS 83:5602-5606, Agrobacterium-mediated transformation (U.S. Pat. Nos. 5,563,055 and 5,981,840), Ochrobactrum-mediated transformation (U.S. Provisional Application 62/211,267), direct gene transfer (Paszkowski et al. (1984) EMBO J 3:2717-2722), and ballistic particle acceleration (see, for example, U.S. Pat. Nos. 4,945,050, 5,879,918, 5,886,244, and 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926), and Lec1 transformation (WO 00/28058). Also see Weissinger et al. (1988) Ann Rev Genet 22:421-477; Sanford et al. (1987) Particulate Science and Technology 5:27-37 (onion); Christou et al. (1988) Plant Physiol 87:671-674 (soybean); McCabe et al. (1988) Bio/Technology 6:923-926 (soybean); Finer & McMullen (1991) In Vitro Cell Dev Biol 27P:175-182 (soybean); Singh et al. (1998) Theor Appl Genet 96:319-324 (soybean); Datta et al. (1990) Biotechnology 8:736-740 (rice); Klein et al. (1988) PNAS 85:4305-4309 (maize); Klein et al. (1988) Biotechnology 6:559-563 (maize); U.S. Pat. Nos. 5,240,855, 5,322,783, and 5,324,646; Klein et al. (1988) Plant Physiol 91:440-444 (maize); Fromm et al. (1990) Biotechnology 8:833-839 (maize); Hooykaas-Van Slogteren et al. (1984) Nature 311:763-764; U.S. Pat. No. 5,736,369 (cereals); Bytebier et al. (1987) PNAS 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Rep 9:415-418 and Kaeppler et al. (1992) Theor. Appl Genet 84:560-566 (whisker-mediated transformation); D′Halluin et al. (1992) Plant Cell 4:1495-1505 (electroporation); Li et al. (1993) Plant Cell Rep 12:250-255 and Christou & Ford (1995) Ann Bot 75:407-413 (rice); Osjoda et al. (1996) Nature Biotechnol 14:745-750 (maize via Agrobacterium tumefaciens); each of which is herein incorporated by reference in its entirety. Transient transformation methods for plants are available in the art and include delivery systems which introduce sequence(s) into a cell such that the sequence(s) are transiently available or expressed in the cell, but not necessarily stably integrated into the genome. Such transient transformation methods include, but are not limited to, microinjection, particle bombardment, Agrobacterium for monocot, modified Agrobacterium-mediated T-DNA transfer for dicots, Ochrobactrum, or viral vectors.

Plant cells that have been transformed to comprise any one or more of the various components (i.e., target locus, site-specific recombinases, recombination sites, polynucleotide construct, or any active variants or fragments thereof) can be grown into whole plants. The cells having a modified genome may be grown into plants in accordance with conventional approaches. See, for example, McCormick et al. (1986) Plant Cell Reports 5:81-84. These regenerated plants may then be pollinated with either the same transformed strain or different strains, and the resulting hybrid having the desired phenotypic characteristic identified. Two or more generations may be grown to ensure that the subject phenotypic characteristic is stably maintained and inherited and then seeds harvested to ensure the desired phenotype or other property has been achieved. In this manner, transformed plants and/or transformed seed having the recited DNA construct stably incorporated into their genome are provided.

Any trait or gene of interest can be integrated at the target locus in the plant cell genome using the methods and compositions provided herein. Such traits include, but are not limited to, traits conferring insect resistance, disease resistance, herbicide tolerance, male sterility, abiotic stress tolerance, altered phosphorus, altered antioxidants, altered fatty acids, altered essential amino acids, altered carbohydrates, or sequences involved in site-specific recombination. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, drought tolerance, waterlogging tolerance, insect resistance, disease tolerance or resistance, herbicide resistance, sterility, grain characteristics or composition (oil, starch, carbohydrate, and/or protein), and commercial products.

An herbicide resistance or tolerance trait confers the ability to tolerate a higher concentration of an herbicide than cells that do not express the protein, or to tolerate a certain concentration of an herbicide for a longer period of time than cells that do not express the protein. Herbicide resistance traits may be introduced into plants by genes coding for resistance to herbicides that act to inhibit the action of acetolactate synthase (ALS), in particular the sulfonylurea-type herbicides, genes coding for resistance to herbicides that act to inhibit the action of glutamine synthase, such as phosphinothricin or basta (e.g., the bar gene), glyphosate (e.g., the EPSP synthase gene and the GAT gene), HPPD inhibitors (e.g., the HPPD gene) or other such genes known in the art. See, for example, U.S. Pat. Nos. 7,626,077, 5,310,667, 5,866,775, 6,225,114, 6,248,876, 7,169,970, 6,867,293, and US20120042412, each of which is herein incorporated by reference in its entirety.

Grain, forage, silage, or processing composition or characteristic traits, including but not limited to such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch.

Sterility genes can also be encoded in a transfer cassette and provide an alternative to physical detasseling. Examples of genes used in such ways include male tissue-preferred genes and genes with male sterility phenotypes such as QM, described in U.S. Pat. No. 5,583,210 which is herein incorporated by reference in its entirety. Other genes include kinases and those encoding compounds toxic to either male or female gametophytic development.

The polynucleotide construct may encode proteins involved in providing disease or pest resistance. By disease resistance, or pest resistance, it is intended that the plants avoid the harmful symptoms that are the outcome of the plant-pathogen interactions. Pest resistance genes may encode resistance to pests that have great yield drag such as rootworm, cutworm, European Corn Borer, and the like. Disease resistance and insect resistance genes such as lysozymes or cecropins for antibacterial protection, or proteins such as defensins, glucanases or chitinases for antifungal protection, or Bacillus thuringiensis endotoxins, protease inhibitors, collagenases, lectins, or glycosidases for controlling nematodes or insects are all examples of useful gene products. Genes encoding disease resistance traits include detoxification genes, such as against fumonosin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like.

The polynucleotide construct can also include a phenotypic marker. A phenotypic marker is screenable or a selectable marker that includes visual markers and selectable markers whether it is a positive or negative selectable marker. Any phenotypic marker can be used. Specifically, a selectable or screenable marker comprises a DNA segment that allows one to identify, or select for or against a molecule or a cell that contains it, often under particular conditions. These markers can encode an activity, such as, but not limited to, production of RNA, peptide, or protein, or can provide a binding site for RNA, peptides, proteins, inorganic and organic compounds or compositions and the like. Examples of selectable markers include, but are not limited to, DNA segments that comprise restriction enzyme sites; DNA segments that encode products which provide resistance against otherwise toxic compounds including antibiotics, such as, spectinomycin, ampicillin, kanamycin, tetracycline, Basta, neomycin phosphotransferase II (NEO) and hygromycin phosphotransferase (HPT)); DNA segments that encode products which are otherwise lacking in the recipient cell (e.g., tRNA genes, auxotrophic markers); DNA segments that encode products which can be readily identified (e.g., phenotypic markers such as β-galactosidase, GUS; fluorescent proteins such as green fluorescent protein (GFP), cyan (CFP), yellow (YFP), red (DsRed), and cell surface proteins); the generation of new primer sites for PCR (e.g., the juxtaposition of two DNA sequences not previously juxtaposed), the inclusion of DNA sequences not acted upon or acted upon by a restriction endonuclease or other DNA modifying enzyme, chemical, etc.; and, the inclusion of a DNA sequence required for a specific modification (e.g., methylation) that allows its identification. Additional selectable markers include genes that confer resistance to herbicidal compounds, such as glufosinate ammonium, bromoxynil, imidazolinones, and 2,4-dichlorophenoxyacetate (2,4-D). See for example, Yarranton (1992) Curr Opin Biotech 3:506-11; Christopherson et al. (1992) PNAS 89:6314-8; Yao et al. (1992) Cell 71:63-72; Reznikoff (1992) Mol Microbiol 6:2419-22; Hu et al. (1987) Cell 48:555-66; Brown et al. (1987) 49:603-12; Figge et al. (1988) Cell 52:713-22; Deuschle et al. (1989) PNAS 86:5400-4; Fuerst et al. (1989) PNAS 86:2549-53; Deuschle et al. (1990) Science 248:480-3; Gossen (1993) Ph.D. Thesis, University of Heidelberg; Reines et al. (1993) PNAS 90:1917-21; Labow et al. (1990) Mol Cell Biol 10:3343-56; Zambretti et al. (1992) PNAS 89:3952-6; Bairn et al. (1991) PNAS 88:5072-6; Wyborski et al. (1991) Nucl Acids Res 19:4647-53; Hillen & Wissman (1989) Topics Mol Struc Biol 10:143-62; Degenkolb et al. (1991) Antimicrob Agents Chemother 35:1591-5; Kleinschnidt et al. (1988) Biochemistry 27:1094-104; Bonin (1993) Ph.D. Thesis, University of Heidelberg; Gossen et al. (1992) PNAS 89:5547-51; Oliva et al. (1992) Antimicrob Agents Chemother 36:913-9; Hlavka et al. (1985) Handbook of Experimental Pharmacology, Vol. 78 (Springer-Verlag, Berlin); Gill et al. (1988) Nature 334:721-724.

Operably linked means there is a functional linkage between two or more elements. For example, an operable linkage between a polynucleotide of interest and a regulatory sequence (i.e., a promoter) is a functional link that allows for expression of the polynucleotide of interest. Operably linked elements may be contiguous or non-contiguous. When used to refer to the joining of two protein coding regions, by operably linked it is intended that the coding regions are in the same reading frame. A transfer cassette may additionally contain at least one additional gene to be co-transformed into the organism. Alternatively, the additional gene(s) can be provided on multiple transfer cassettes. Such a transfer cassette is provided with a plurality of restriction sites and/or recombination sites for insertion of a recombinant polynucleotide to be under the transcriptional regulation of the regulatory regions. A transfer cassette may additionally contain a selectable marker gene.

A transfer cassette can include in the 5′-3′ direction of transcription, a transcriptional and translational initiation region (i.e., a promoter), a recombinant polynucleotide provided herein, and a transcriptional and translational termination region (i.e., termination region) functional in plants. The regulatory regions (i.e., promoters, transcriptional regulatory regions, and translational termination regions) and/or a polynucleotide provided herein may be native/analogous to the host cell or to each other. Alternatively, the regulatory regions and/or a polynucleotide provided herein may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. Alternatively, the regulatory regions and/or a recombinant polynucleotide provided herein may be entirely synthetic.

The termination region may be native with the transcriptional initiation region, may be native with the operably linked recombinant polynucleotide, may be native with the plant host, or may be derived from another source (i.e., foreign or heterologous) to the promoter, the recombinant polynucleotide, the plant host, or any combination thereof. Convenient termination regions are available from the Ti-plasmid of A. tumefaciens, such as the octopine synthase and nopaline synthase termination regions. See also Guerineau et al. (1991) Mol Gen Genet 262:141-144; Proudfoot (1991) Cell 64:671-674; Sanfacon et al. (1991) Genes Dev 5:141-149; Mogen et al. (1990) Plant Cell 2:1261-1272; Munroe et al. (1990) Gene 91:151-158; Ballas et al. (1989) Nucl Acids Res 17:7891-7903; and Joshi et al. (1987) Nucl Acids Res 15:9627-9639.

A number of promoters can be used in the constructs and transfer cassettes provided herein. The promoters can be selected based on the desired outcome. It is recognized that different applications can be enhanced by the use of different promoters in a transfer cassette to modulate the timing, location and/or level of expression of the polynucleotide of interest. Such expression constructs may also contain, if desired, a promoter regulatory region (e.g., one conferring inducible, constitutive, environmentally- or developmentally-regulated, or cell- or tissue-specific/selective expression), a transcription initiation start site, a ribosome binding site, an RNA processing signal, a transcription termination site, and/or a polyadenylation signal.

Examples of constitutive promoters include the cauliflower mosaic virus (CaMV) 35S transcription initiation region, the 1′- or 2′-promoter derived from T-DNA of Agrobacterium tumefaciens, the ubiquitin 1 promoter, the Smas promoter, the cinnamyl alcohol dehydrogenase promoter (U.S. Pat. No. 5,683,439), the Nos promoter, the pEmu promoter, the rubisco promoter, the GRP1-8 promoter and other transcription initiation regions from various plant genes known to those of skill. If low level expression is desired, weak promoter(s) may be used. Weak constitutive promoters include, for example, the core promoter of the Rsyn7 promoter (WO 99/43838 and U.S. Pat. No. 6,072,050), the core 35S CaMV promoter, and the like. Other constitutive promoters include those disclosed in, for example, U.S. Pat. Nos. 5,608,149; 5,608,144; 5,604,121; 5,569,597; 5,466,785; 5,399,680; 5,268,463; 5,608,142and U.S. Pat. No. 6,177,611, each of which is herein incorporated by reference in its entirety.

Examples of inducible promoters include the Adh1 promoter which is inducible by hypoxia or cold stress, the Hsp70 promoter which is inducible by heat stress, the PPDK promoter and the pepcarboxylase promoter which are both inducible by light. Also useful are promoters which are chemically inducible, such as the In2-2 promoter which is safener induced (U.S. Pat. No. 5,364,780), the ERE promoter which is estrogen induced, and the Axigl promoter which is auxin induced and tapetum specific but also active in callus (WO2002006499).

Examples of promoters under developmental control include promoters that initiate transcription preferentially in certain tissues, such as leaves, roots, fruit, seeds, or flowers. An exemplary promoter is the anther specific promoter 5126 (U.S. Pat. Nos. 5,689,049 and 5,689,051). Examples of seed-preferred promoters include, but are not limited to, 27 kD gamma zein promoter and waxy promoter, Boronat et al. (1986) Plant Sci 47:95-102; Reina et al. (1990) Nucl Acids Res 18:6426; and Kloesgen et al. (1986) Mol Gen Genet 203:237-244. Promoters that express in the embryo, pericarp, and endosperm are disclosed in U.S. Pat. No. 6,225,529 and WO 00/12733. The disclosures for each of these are incorporated herein by reference in their entirety.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-la promoter, which is activated by salicylic acid. Other chemical-regulated promoters of interest include steroid-responsive promoters (see, e.g., the glucocorticoid-inducible promoter in Schena et al. (1991) PNAS 88:10421-10425; and, McNellis et al. (1998) Plant J 14:247-257) and tetracycline-inducible and tetracycline-repressible promoters (see, e.g., Gatz et al. (1991) Mol Gen Genet 227:229-237; and U.S. Pat. Nos. 5,814,618 and 5,789,156), herein incorporated by reference.

Tissue-preferred promoters can be utilized to target enhanced expression of a polynucleotide of interest within a particular plant tissue. Tissue-preferred promoters are known in the art. See, for example, Yamamoto et al. (1997) Plant J 12:255-265; Kawamata et al. (1997) Plant Cell Physiol 38:792-803; Hansen et al. (1997) Mol Gen Genet 254:337-343; Russell et al. (1997) Transgenic Res 6:157-168; Rinehart et al. (1996) Plant Physiol 112:1331-1341; Van Camp et al. (1996) Plant Physiol 112:525-535; Canevascini et al. (1996) Plant Physiol 112:513-524; Yamamoto et al. (1994) Plant Cell Physiol 35:773-778; Lam (1994) Results Probl Cell Differ 20:181-196; Orozco et al. (1993) Plant Mol Biol 23:1129-1138; Matsuoka et al. (1993) PNAS 90:9586-9590; and Guevara-Garcia et al. (1993) Plant J 4:495-505. Such promoters can be modified, if necessary, for weak expression.

Standard techniques for the construction of the vectors of the present disclosure are well-known to those of ordinary skill in the art and can be found in such references as Sambrook et al., Molecular Cloning: A Laboratory Manual, 2nd ed. (Cold Spring Harbor, New York, 1989). A variety of strategies are available for ligating fragments of DNA, the choice of which depends on the nature of the termini of the DNA fragments and which choices can be readily made by those of skill in the art.

Agrobacterium-mediated site-specific integration is widely believed by those skilled in the art to be a highly inefficient process and particularly so in monocots. It is believed that three possible explanations may account for this inefficiency.
1) To date, studies have indicated that linear ssDNA are incompatible substrates for recombinases (single-stranded T-DNA is not a substrate for recombinase systems like Cre/Lox, Tinland et al. (1994) PNAS 91:8000-8004; Louwerse et al. (2007) Plant Physiol 145:1282-1293), while dsDNA molecules are ideal substrates for recombinases and recombination.
2) Further, circular DNA molecules are preferred for targeting the DNA to a single chromosomal site. Although it is widely accepted that single-stranded T-DNA molecules are converted into double stranded molecules prior to random integration, the frequency of circular intermediates formed from the T-strands is very low or remains unclear, since border ligations are very inefficient (Zhao et al. (2003) Plant J 33:149-159; Bundock et al. (1995) EMBO J 14:3206-3214).
3) One would also expect that the recombination substrates, enzymes, and other factors need to be present at a sufficient concentration for efficient reaction kinetics. The amount of the donor DNA, enzyme, and any accessory element, such as cell proliferation factor(s), in the cells is critical for efficient recombination-mediated cassette exchange (RMCE). It is known that an Agrobacterium delivery system provides only a limited amount of DNA per infected cell because of the low copy number of the binary plasmid (i.e., ˜15-20 copies per Agrobacterium cell (Oltmanns et al. (2010) Plant Physiol 152:1158-1166), that might extrapolate to 100 copies or less ssDNA/cell if there are multiple Agrobacteria attached, and the majority of which DNA degrades prior to integration). Additionally, using a higher inoculum of Agrobacterium often results in poor tissue response resulting from cell death or pathogenesis.

On the contrary, delivery by particle bombardment can deliver several fold higher amounts of DNA per cell, and the amount of DNA delivered on the particles can be titrated to improve the RMCE efficiency in the recipient cell.

Even though Agrobacterium-mediated SSI was demonstrated many years ago (Vergunst & Hooykas (1998) Plant Mol. Biol 38:393-406, Vergunst et al. (1998) Nucl Acids Res 26:2729-2734), the low frequency of transformation meant the method was impractical, and therefore gene gun delivery has remained the method of choice for SSI in plants until the present. Previous published work on Agrobacterium-mediated SSI showed the process was very inefficient (Vergunst et al. (1998) Nucl Acids Res 26:2729-2734; Vergunst & Hooykaas (1998) Plant Mol. Biol 38:393-406), particularly when compared to site-specific integration rates using the particle gun for DNA delivery (Albert et al. (1995) Plant J 7:649-659; Day et al. (2000) Genes Dev 14:2869-2880; Srivastava & Ow (2001) Mol Breeding 8:345-350; Li et al. (2009) Plant Physiol 151:1087-1095). To our knowledge, there is no work demonstrating successful Agrobacterium-mediated SSI in monocot species. As exemplified herein, providing one or more cell proliferation factors, such as developmental genes, in a controlled manner (limited amounts), use of highly virulent strains of Agrobacteria for increased T-DNA delivery and improved vector designs for Agrobacterium-mediated transformation appears to overcome the inefficiencies associated with Agrobacterium-mediated methods previously tested for SSI and avoids or mitigates the stresses that accompany particle gun delivery.

### EXAMPLES

The following Examples are offered by way of illustration and not by way of limitation.

### Example 1

### Culture Media

Various media are referenced in the Examples for use in transformation and cell culture. These media descriptions are found below in Table2.

TABLE 2
Media formations for maize transformation, selection and regeneration

| | Units | | | | | | | | | |
|---|---|---|---|---|---|---|---|---|---|---|
| | per | | | | | | | | | |
| Medium components | liter | 12V | 810I | 700 | 710I | 605J | 605T | 289Q | 605W | 289M |
| MS BASAL SALT MIXTURE | g | | | 4.3 | 4.3 | 4.3 | 4.3 | 4.3 | 4.3 | 4.3 |
| N6 MACRONUTRIENTS 10X | ml | | | | | 60.0 | 60.0 | | 60.0 | |
| POTASSIUM NITRATE | g | | | | | 1.7 | 1.7 | | 1.7 | |
| B5H MINOR SALTS 1000X | ml | | | | | 0.6 | 0.6 | | 0.6 | |
| NaFe EDTA FOR B5H 100X | ml | | | | | 6.0 | 6.0 | | 6.0 | |
| ERIKSSON'S VITAMINS | ml | | | | | 0.4 | 0.4 | | 0.4 | |
| 1000X | | | | | | | | | | |
| S&H VITAMIN STOCK 100X | ml | | | | | 6.0 | 6.0 | | 6.0 | |
| THIAMINE•HCL | mg | | | 1.0 | 1.0 | 0.2 | 0.2 | | 0.2 | |
| L-PROLINE | g | | | | 0.7 | 2.0 | 2.0 | 0.7 | 2.0 | 0.7 |
| CASEIN HYDROLYSATE | g | | | | | 0.3 | 0.3 | | 0.3 | |
| (ACID) | | | | | | | | | | |
| SUCROSE | g | | | 68.5 | 20.0 | 20.0 | 20.0 | 60.0 | 20.0 | 60.0 |
| GLUCOSE | g | 5.0 | | 36.0 | 10.0 | 0.6 | 0.6 | | 10.0 | |
| MALTOSE | g | | | | | | | | | |
| 2,4-D | mg | | | 1.5 | 2.0 | 0.8 | 0.8 | | 0.8 | |
| AGAR | g | 15.0 | 15.0 | | 8.0 | 6.0 | 6.0 | 8.0 | | |
| PHYTAGEL | g | | | | | | | | 3.5 | |
| DICAMBA | g | | | | | 1.2 | 1.2 | | 1.2 | |
| SILVER NITRATE | mg | | | | | 3.4 | 3.4 | | | |
| AGRIBIO Carbenicillin | mg | | | | | 100.0 | | | 100.0 | |
| Timentin | mg | | | | | | 150.0 | 150.0 | | |
| Cefotaxime | mg | | | | | | 100.0 | 100.0 | | |
| MYO-INOSITOL | g | | | 0.1 | 0.1 | | | 0.1 | | 0.1 |
| NICOTINIC ACID | mg | | | 0.5 | 0.5 | | | | | |
| PYRIDOXINE•HCL | mg | | | 0.5 | 0.5 | | | | | |
| VITAMIN ASSAY | g | | | 1.0 | | | | | | |
| CASAMINO ACIDS | | | | | | | | | | |
| MES BUFFER | g | | | | 0.5 | | | | | |
| ACETOSYRINGONE | uM | | | | 100.0 | | | | | |
| ASCORBIC ACID 10 MG/ML | mg | | | | 10.0 | | | | | |
| (7S) | | | | | | | | | | |
| MS VITAMIN STOCK SOL. | ml | | | | | | | 5.0 | | 5.0 |
| ZEATIN | mg | | | | | | | 0.5 | | 0.5 |
| CUPRIC SULFATE | mg | | | | | | | 1.3 | 0.05 | 1.3 |
| IAA 0.5 MG/ML (28A) | ml | | | | | | | 2.0 | | 2.0 |
| ABA 0.1 mm | ml | | | | | | | 1.0 | | 1.0 |
| THIDIAZURON | mg | | | | | | | 0.1 | | 0.1 |
| AGRIBIO Carbenicillin | mg | | | | | | | 100.0 | | 100.0 |
| PPT(GLUFOSINATE-NH4) | mg | | | | | | | | | |
| BAP | mg | | | | | | | 1.0 | 0.1 | |
| YEAST EXTRACT (BD Difco) | g | | 5.0 | | | | | | | |
| PEPTONE | g | | 10.0 | | | | | | | |
| SODIUM CHLORIDE | g | | 5.0 | | | | | | | |
| SPECTINOMYCIN | mg | 50.0 | 100.0 | | | | | | | |
| FERROUS SULFATE•7H20 | ml | 2.0 | | | | | | | | |
| AB BUFFER 20X (12D) | ml | 50.0 | | | | | | | | |
| AB SALTS 20X (12E) | ml | 50.0 | | | | | | | | |
| Benomyl | mg | | | | | | | | | |
| pH | | 5.6 | 5.6 | 5.6 | 5.6 | 5.6 | 5.6 | 5.6 | 5.6 | 5.6 |

| | Units | | | | | | | | | | |
|---|---|---|---|---|---|---|---|---|---|---|---|
| | per | | | | | | | | | | |
| Medium components | liter | 289R | 13158H | 13224B | 13266K | 272X | 272V | 13158 | 13265A | 13152T | 13152Z |
| MS BASAL SALT MIXTURE | g | 4.3 | 4.3 | | 4.3 | 4.3 | 4.3 | 4.3 | 4.3 | 4.3 | 4.3 |
| N6 MACRONUTRIENTS 10X | ml | | | 4.0 | 60.0 | | | | 60.0 | | |
| POTASSIUM NITRATE | g | | | | 1.7 | | | | 1.7 | | |
| B5H MINOR SALTS 1000X | ml | | | | 0.6 | | | | 0.6 | | |
| NaFe EDTA FOR B5H 100X | ml | | | | 6.0 | | | | 6.0 | | |
| ERIKSSON'S VITAMINS | ml | | | 1.0 | 0.4 | | | | 0.4 | | |
| 1000X | | | | | | | | | | | |
| S&H VITAMIN STOCK 100X | ml | | | | 6.0 | | | | 6.0 | | |
| THIAMINE•HCL | mg | | | 0.5 | 0.5 | | | | 0.5 | 1.0 | 1.0 |
| L-PROLINE | g | 0.7 | 0.7 | 2.9 | 2.0 | | | | 2.0 | 0.7 | 0.7 |
| CASEIN HYDROLYSATE | g | | | | 0.3 | | | | 0.3 | 1.0 | 1.0 |
| (ACID) | | | | | | | | | | | |
| SUCROSE | g | 60.0 | 60.0 | 190.0 | 20.0 | 40.0 | 40.0 | 40.0 | 20.0 | | |
| GLUCOSE | g | | | | 0.6 | | | | 10.0 | | |
| MANNOSE | g | | | | | | | | | 12.5 | 12.5 |
| MALTOSE | g | | | | | | | | | 5.0 | 5.0 |
| 2,4-D | mg | | | | 1.6 | | | | 1.6 | 1.0 | 1.0 |
| AGAR | g | | 8.0 | 6.4 | 6.0 | 6.0 | 6.0 | 6.0 | | | |
| PHYTAGEL | g | | | | | | | | 3.5 | | |
| DICAMBA | g | | | | 1.2 | | | | 1.2 | | |
| SILVER NITRATE | mg | | | 8.5 | 1.7 | | | | | | |
| AGRIBIO Carbenicillin | mg | | | | 2.0 | | | | | 100 | |
| Timentin | mg | 150.0 | 150.0 | | | | | | 150.0 | | 150.0 |
| Cefotaxime | mg | 100.0 | 100.0 | 25 | 25 | | | | 100.0 | | 100.0 |
| MYO-INOSITOL | g | 0.1 | 0.1 | | | 0.1 | 0.1 | 0.1 | | 0.25 | 0.25 |
| NICOTINIC ACID | mg | | | | | | | | | | |
| PYRIDOXINE•HCL | mg | | | | | | | | | | |
| VITAMIN ASSAY | g | | | | | | | | | | |
| CASAMINO ACIDS | | | | | | | | | | | |
| MES BUFFER | g | | | | | | | | | | |
| ACETOSYRINGONE | uM | | | | | | | | | | |
| ASCORBIC ACID 10 MG/ML | mg | | | | | | | | | | |
| (7S) | | | | | | | | | | | |
| MS VITAMIN STOCK SOL. | ml | 5.0 | 5.0 | | | 5.0 | 5.0 | 5.0 | | | |
| ZEATIN | mg | 0.5 | 0.5 | | | | | | | | |
| CUPRIC SULFATE | mg | 1.3 | 1.3 | | | | | | 0.05 | 1.2 | 1.2 |
| IAA 0.5 MG/ML (28A) | ml | 2.0 | 2.0 | | | | | | | | |
| ABA 0.1 mm | ml | 1.0 | 1.0 | | | | | | | | |
| THIDIAZURON | mg | 0.1 | 0.1 | | | | | | | | |
| AGRIBIO Carbenicillin | mg | | | | | | | | | | |
| PPT(GLUFOSINATE-NH4) | mg | | | | | | | | | | |
| BAP | mg | | | | | | | | 0.1 | 0.5 | 0.5 |
| YEAST EXTRACT (BD Difco) | g | | | | | | | | | | |
| PEPTONE | g | | | | | | | | | | |
| SODIUM CHLORIDE | g | | | | | | | | | | |
| SPECTINOMYCIN | mg | | | | | | | | | | |
| FERROUS SULFATE•7H20 | ml | | | | | | | | | | |
| AB BUFFER 20X (12D) | ml | | | | | | | | | | |
| AB SALTS 20X (12E) | ml | | | | | | | | | | |
| Benomyl | mg | | | | | | | 100.0 | | | |
| pH | | 5.6 | 5.6 | 5.6 | 5.6 | 5.6 | 5.6 | 5.6 | 5.6 | 5.6 | 5.6 |

### Example 2

### Production of Transgenic Maize Events Using Agrobacterium Transformation

Agrobacterium tumefaciens harboring a binary donor vector containing a phosphomannose-isomerase selectable marker (PMI) in a promoter trap and a reporter marker (DsRed) was streaked out from a −80° C. frozen aliquot onto solid PHI-L medium and cultured at 28° C. in the dark for 2-3 days. PHI-L media comprised 25 ml/L stock solution A, 25 ml/L stock solution B, 450.9 ml/L stock solution C and spectinomycin added to a concentration of 50 mg/L in sterile ddH2O (stock solution A: K2HPO4 60.0 g/L, NaH2PO4 20.0 g/L, adjust pH to 7.0 with KOH and autoclave; stock solution B: NH4Cl 20.0 g/L, MgSO4-7H2O 6.0 g/L, KCl 3.0 g/L, CaCl2 0.20 g/L, FeSO4.7H2O 50.0 mg/L, autoclave; stock solution C: glucose 5.56 g/L, agar 16.67 g/L and autoclave). Agrobacterium used for transformation were grown on solid medium, and/or in liquid culture, as described below.

### A. Growing Agrobacterium on Solid Medium

A single colony or multiple colonies were picked from the master plate and streaked onto a plate containing PHI-M medium (yeast extract (Difco) 5.0 g/L; peptone (Difco)10.0 g/L; NaCl 5.0 g/L; agar (Difco) 15.0 g/L; pH 6.8, containing 50 mg/L spectinomycin), and incubated at 28° C. in the dark for 1-2 days.

Five mL Agrobacterium infection medium (PHI-A: CHU(N6) basal salts (Sigma C-1416) 4.0 g/L, Eriksson's vitamin mix (1000×, Sigma-1511) 1.0 ml/L; thiamine-HCl 0.5 mg/L (Sigma); 2,4-dichlorophenoxyacetic acid (2,4-D, Sigma) 1.5 mg/L; L-proline (Sigma) 0.69 g/L; sucrose (Mallinckrodt) 68.5 g/L; glucose (Mallinckrodt) 36.0 g/L; pH 5.2; or, PHI-I: MS salts (GIBCO BRL) 4.3 g/L; nicotinic acid (Sigma) 0.5 mg/L; pyridoxine-HCl (Sigma) 0.5 mg/L; thiamine-HCl 1.0 mg/L; myo-inositol (Sigma) 0.10 g/L; vitamin assay casamino acids (Difco Lab) 1 g/L; 2, 4-D 1.5 mg/L; sucrose 68.50 g/L; glucose 36.0 g/L; adjust pH to 5.2 w/KOH and filter-sterilize) and 5 μL of 100 mM 3′-5′-dimethoxy-4′-hydroxyacetophenone (acetosyringone) were added to a 14 mL tube. About 3 full loops of Agrobacterium were suspended in the tube which was then vortexed to make an even suspension. One mL of the suspension was transferred to a spectrophotometer tube and the optical density (OD) of the suspension was adjusted to 0.35-2.0 at 550 nm to yield an Agrobacterium concentration of about 0.5-2.0×10⁹ cfu/mL. The final Agrobacterium suspension was aliquoted into 2 mL microcentrifuge tubes, each containing 1 mL of the suspension. The suspensions were then used for transformations as soon as possible.

### B. Growing Agrobacterium on Liquid Medium

One day before infection, a 125 mL flask was set up with 30 mL of 557A media (10.5 g/L potassium phosphate dibasic, 4.5 g/L potassium phosphate monobasic, 1.0 g/L ammonium sulfate, 0.5 g/L sodium citrate dihydrate, 0.2% (w/v) sucrose, 1 mM magnesium sulfate) with 30 μL each of spectinomycin (50 mg/mL) and acetosyringone (20 mg/mL). One-half loopful of Agrobacterium was suspended into each flask and grown overnight at 28° C. with shaking at 200 rpm. The Agrobacterium culture was centrifuged at 5000 rpm for 10 min. The supernatant was removed and the Agrobacterium infection medium+acetosyringone solution was added. The bacteria were resuspended by vortexing and the OD of Agrobacterium suspension was adjusted to 0.35-2.0 at 550 nm.

### Maize Transformation:

Ears of a maize (Zea mays L.) cultivar, HC69, were surface-sterilized for 15-20 min in 20% (v/v) bleach (5.25% sodium hypochlorite) plus 1 drop of Tween 20 followed by 3 washes in sterile water. Immature embryos (IEs), typically 1.5-1.8 mm, were isolated from ears and were placed in 2 ml of the Agrobacterium infection medium+acetosyringone solution. The solution was drawn off and 1 ml of Agrobacterium suspension was added to the embryos, vortexed for 5-10 seconds, and then incubated 5 min at room temperature. The suspension of Agrobacterium and embryos were poured onto co-cultivation 7101 medium. Any embryos left in the tube were transferred to the plate using a sterile spatula. The Agrobacterium suspension was drawn off and the embryos were placed axis side down on the media. The plate was sealed with PARAFILM® film (moisture resistant flexible plastic, available at Bemis Company, Inc., 1 Neenah Center 4^{th} floor, PO Box 669, Neenah, Wis. 54957) and incubated in the dark at 21° C. for 1-3 days of co-cultivation.

Embryos were transferred to resting 13265A medium without selection. Three to 7 days later, they were transferred to green tissue induction medium (DBC3: 4.3 g/L MS salts, 30 g/L maltose, 1 mg/mL thiamine-HC1, 0.25 g/L myo-inositol, 1 g/L N-Z-amine-A (casein hydrolysate), 0.69 g/L proline, 4.9 μM CuSO₄, 1.0 mg/L 2,4-D, 0.5 mg/L BAP; pH 5.8 3.5 g/L Phytagel) supplemented with mannose (12.5 g/L). Three weeks after the first round of selection, cultures were transferred to fresh green tissue induction medium containing mannose at 3- to 4-week intervals. Once transformed, transgenic green tissues were selected and cultured essentially as described in U.S. Pat. No. 7,102,056, U.S. Pat. No. 8,404,930, and publication US20130055472, each of which is herein incorporated by reference in its entirety.

### Example 3

### Generation of Target Loci For Agro SSI

A site-specific integration (SSI) target locus was created in maize inbreds, HC69 and PH184C, using Agrobacterium mediated immature embryo transformation essentially as described in U.S. Pat. No. 6,187,994, herein incorporated by reference in its entirety. A target locus (gDNA-Pro-RS1-RS2-gDNA) (see Table 1) was generated in HC69 and comprised in operable linkage flanked by HC69 genomic DNA a promoter trap (ZmUbiPro; SEQ ID NO: 2), a first recombination site (FRT1; SEQ ID NO: 7), a selectable marker gene (NptII; SEQ ID NO: 39), a terminator (PINII; SEQ ID NO: 40), a promoter (ZmUbiPro; SEQ ID NO: 2), a fluorescent marker gene (AmCyan (CFP); SEQ ID NO: 41), a terminator (PINII; SEQ ID NO: 40), and a second non-identical recombination site (FRT87; SEQ ID NO: 8). Lines comprising the promoter trap target locus were generated by transforming HC69 with a T-DNA construct comprising PHP64484: ZmUbiPro-FRT1-NptII::PinII+ZmUbiPro::AmCyan (CFP)::PinII-FRT87 (SEQ ID NO: 1).

This target locus (gDNA-Pro-RS1-RS2-gDNA) (see Table 1) was used for retransformation. Successful retransformation using the Transfer Cassettes disclosed here in resulted in the identification and characterization of SSI events. Successful retransformation resulted in the replacement of the selectable marker gene (NptII; SEQ ID NO: 39) and the fluorescent marker gene (AmCyan (CFP); SEQ ID NO: 41) at the target locus (gDNA-Pro-RS1-RS2-gDNA) (see Table 1) with the polynucleotide construct used for retransformation as described below (see Example 5).

### Example 4

### Binary Vector Designs for Agro-Mediated Site-Specific Integration in Plants

Transfer cassettes were generated for use in further experiments. Specifically, a Transfer Cassette A: RB-RS1-POI-RS2-R1-LB (PHP60577; SEQ ID NO: 12) (see Table 1 A) was generated and comprised in operable linkage a first recombination site (FRT1; SEQ ID NO: 7), a polynucleotide construct comprising a promoterless PMI gene (SEQ ID NO: 42), a terminator (PINII; SEQ ID NO: 40), a constitutive promoter (ZmUbiPro; SEQ ID NO: 2) driving expression of a reporter gene (DsRed; SEQ ID NO: 3) and a terminator (PINII; SEQ ID NO: 40), a second recombination site (FRT87; SEQ ID NO: 8), and a recombinase construct comprising a constitutive promoter (ZmUbiPro; SEQ ID NO: 2), and a terminator (PINII; (SEQ ID NO: 40). Transfer Cassette A has the recombinase construct located downstream of the polynucleotide construct. One skilled in the art would appreciate that the recombinase construct could be located upstream of the polynucleotide construct.

Transfer Cassette F: RB-R1-RS1-POI-RS2-RS3-R2-CPF-RS3-LB (PHP62258; SEQ ID NO: 14) (see Table 1 F) was generated and comprised in operable linkage a recombinase construct comprising a constitutive promoter (ZmUbiPro; SEQ ID NO: 2), the FLP gene (comprising FLP exonl; SEQ ID NO: 10 and FLP exon2; SEQ ID NO: 11), and a terminator (PINII; SEQ ID NO: 40), a first recombination site (FRT1; SEQ ID NO: 7), a polynucleotide construct comprising a promoterless PMI gene (SEQ ID NO: 42), a terminator (PINII; SEQ ID NO: 40), a constitutive promoter (ZmUbiPro; SEQ ID NO: 2) driving expression of a reporter gene (DsRed; SEQ ID NO: 3) and a terminator (PINII; SEQ ID NO: 40), a second recombination site (FRT87; SEQ ID NO: 8), a third recombination site (LOXP; SEQ ID NO: 31), a second recombinase construct comprising a promotor (Rab17PRO; SEQ ID NO: 43), a CRE recombinase gene comprising (CRE exon 1; SEQ ID NO: 29 and CRE exon 2; (SEQ ID NO: 30), and a terminator (PINII; SEQ ID NO:), an excisible cell proliferation factor construct comprising a NosPro::WUS::PINII (SEQ ID NO: 32) and ZmUbiPro::BBM::PINII (SEQ ID NO: 33), and a third recombination site (LOXP; SEQ ID NO: 31). Transfer Cassette F has the second recombinase construct located within the third recombination sites and downstream of the polynucleotide construct. One skilled in the art would appreciate that the second recombinase construct could be located outside the third recombination sites and upstream of the polynucleotide construct and upstream or downstream of the cell proliferation construct.

Transfer Cassette F1: RB-RS3-R2-CPF-RS3-RS1-POI-RS2-R1-LB (PHP62558; SEQ ID NO: 13) (see Table 1 F1) was generated and comprised in operable linkage a third recombination site (LOXP; SEQ ID NO: 31), a second recombinase construct comprising a promotor (Rabl7PRO; (SEQ ID NO: 43), a CRE recombinase gene comprising (CRE exon 1; SEQ ID NO: 29 and CRE exon 2; (SEQ ID NO: 30), and a terminator (PINII; SEQ ID NO: 40), an excisible cell proliferation factor construct comprising NosPro::WUS::PINII (SEQ ID NO: 32) and ZmUbiPro::BBM::PINII (SEQ ID NO: 33), and a third recombination site (LOXP; SEQ ID NO: 31), a first recombination site (FRT1; SEQ ID NO: 7), a polynucleotide construct comprising a promoterless PMI gene (SEQ ID NO: 42), a terminator (PINII; SEQ ID NO: 40), a constitutive promoter (ZmUbiPro; SEQ ID NO: 2) driving expression of a reporter gene (DsRed; SEQ ID NO: 3) and a terminator (PINII; SEQ ID NO: 40), a second recombination site (FRT87; SEQ ID NO: 8), and a first recombinase construct comprising a constitutive promoter (ZmUbiPro; SEQ ID NO: 2), the FLP gene (comprising FLP exonl; SEQ ID NO: 10 and FLP exon2; SEQ ID NO: 11), and a terminator (PINII; SEQ ID NO: 40). Transfer Cassette F1 has the second recombinase construct located within the third recombination sites and upstream of the polynucleotide construct. One skilled in the art would appreciate that the second recombinase construct could be located outside the third recombination sites and downstream of the polynucleotide construct and upstream or downstream of the cell proliferation construct.

Transfer Cassette S: comprising Transfer Cassette 1: RB-RS1-POI-RS2-R1-LB and Transfer Cassette 2: RB-RS3-R2-CPF-RS3-LB (see Table 1 S) was generated. Transfer Cassette 1 and Transfer Cassette 2 of Transfer Cassette S were delivered on two independent binary vectors to plant cells from the same Agrobacterium strain. One skilled in the art would appreciate that Transfer Cassette 1 and Transfer Cassette 2 of Transfer Cassette S could be delivered from a single binary vector. One skilled in the art would also appreciate that different Agrobacterium strains could be used for delivering Transfer Cassette 1 and Transfer Cassette 2 of Transfer Cassette S.

Transfer Cassette 1 of Transfer Cassette S: RB-RS1-POI-RS2-R1-LB (PHP60577; SEQ ID NO: 12) (see Table 1 S) comprised in operable linkage a first recombination site (FRT1; SEQ ID NO: 7), a polynucleotide construct comprising a promoterless PMI gene (SEQ ID NO: 42), a terminator (PINII; SEQ ID NO: 40), a constitutive promoter (ZmUbiPro; SEQ ID NO: 2) driving expression of a reporter gene (DsRed; SEQ ID NO: 3) and a terminator (PINII; SEQ ID NO: 40), a second recombination site (FRT87; SEQ ID NO: 8), and a recombinase construct comprising a constitutive promoter (ZmUbiPro; SEQ ID NO: 2), the FLP gene (comprising FLP exonl; SEQ ID NO: 10 and FLP exon2; SEQ ID NO: 11), and a terminator (PINII; SEQ ID NO: 40). Transfer Cassette 1 of Transfer Cassette S has a unique antibiotic selectable marker on the vector backbone (spectinomycin (SEQ ID NO: 16)). Transfer Cassette 1 of Transfer Cassette S has the recombinase construct located downstream of the polynucleotide construct. One skilled in the art would appreciate that the recombinase construct could be located upstream of the polynucleotide construct.

Transfer Cassette 2 of Transfer Cassette S: RB-RS3-R2-CPF-RS3-LB (PHP44542 (SEQ ID NO: 15) (see Table 1 S) comprised in operable linkage a third recombination site (LOXP; SEQ ID NO: 31), a second recombinase construct comprising a promotor (Rab17PRO; (SEQ ID NO: 43), a CRE recombinase gene comprising (CRE exon 1; SEQ ID NO: 29 and CRE exon 2; SEQ ID NO: 30), and a terminator (PINII; SEQ ID NO: 40), an excisible cell proliferation factor construct comprising NosPro::WUS::PINII (SEQ ID NO: 32) and ZmUbiPro::BBM::PINII (SEQ ID NO: 33), and a third recombination site (LOXP; SEQ ID NO: 31). Transfer Cassette 2 of Transfer Cassette S has a different antibiotic selectable marker on the vector backbone (kanamycin; SEQ ID NO: 17). Transfer Cassette 2 of Transfer Cassette S has the second recombinase construct located within the third recombination sites. One skilled in the art would appreciate that the second recombinase construct could be located outside the third recombination sites and upstream or downstream of the cell proliferation construct.

### Example 5

### Testing of Agrobacterium Strains AGL1 and LBA4404 for Site-Specific Integration

Two different Agrobacterium strains, a nopaline type Agrobacterium strain C58 (AGL1; pTiB0542) and an octopine type Agrobacterium strain TiAch 5 (LBA4404; pAL4404) were used in retransformations to evaluate site-specific integration (SSI) into the target locus (gDNA-Pro-RS1-RS2-gDNA) (see Table 1) generated in maize cultivar HC69 (see Example 3). Transfer Cassette A: RB-RS1-POI-R52-R1-LB (PHP60577; SEQ ID NO: 12) (see Table 1 A) was used to determine the SSI frequency at the target locus (gDNA-Pro-RS1-RS2-gDNA) (see Table 1) in maize cultivar HC69.

Following retransformation of immature embryos containing the target locus with Agrobacterium strains AGL1 or LBA4404 containing Transfer Cassette A: RB-RS1-POI-RS2-R1-LB (PHP60577; SEQ ID NO: 12). SSI events were selected on a media supplemented with mannose (PMI selection as described in U.S. Pat. Nos. 5,994,629 and 5,767,378 each of which is incorporated herein by reference in its entirety). Putative callus events were identified by culturing the retransformed embryos on media supplemented with mannose, and through the expression of DsRed. Transformants wherein the target locus (NptII-CFP genes) was replaced with the polynucleotide construct (PMI/DsRed genes) were identified by their callus morphology and detection of the fluorescent gene (DsRed) expression. These events were regenerated and the T0 plants were analyzed using standard qPCR assays. Table 3 shows the transformation frequency and frequency of site-specific recombination events recovered from maize inbred line HC69. Table 3 also shows the results of the precise site-specific integration event frequency. The transformation frequency of the AGL1 strain was higher than that of the LBA4404 strain.

TABLE 3
Transformation frequency and SSI frequency
using Transfer Cassette A

| | | # events | | | Frequency | | |
|---|---|---|---|---|---|---|---|
| Strain | #embryos | Callus | T0 | SSI | Callus | T0 | SSI |
| AGL1 | 3376 | 13 | 9 | 4 | 0.4% | 0.27% | 0.12% |
| LBA4404 | 4015 | 12 | 2 | 0 | 0.3% | 0.05% | 0% |

Precise site-specific integration events were identified using multiplex PCR assays to detect the presence/absence of random Agrobacterium T-DNA, and qPCR/PCR assays to confirm the excision of the target gene (NptII) at the target locus, and integration of the polynucleotide construct (PMI and DsRed) at the target locus flanked by the first recombination site and the second non-identical recombination site (FRT1/FRT87). These results are described in the Table 4. A precise SSI event was identified using the following criteria:

1) a single intact copy of the polynucleotide construct (PMI and DsRed genes);

2) the absence of the target gene (NptII) at the target locus and the absence of the FLP recombinase construct;

3) the presence of the FRT1 and FRT87 junctions; and,

4) the absence of vector backbone (see Table 4).

All other events which did not meet the above criteria were identified as imprecise SSI events (see Table 4).

Table 4 summarizes the PCR analysis results to identify precise SSI events. qPCR assays were developed to determine the absence (excision) of target gene (NptII) at the target locus, integration of the polynucleotide construct (PMI and DsRed genes) at the target locus, the absence of the FLP recombinase construct, and the presence of the first recombination site and the second non-identical recombination site (FRT1/FRT87) at the target locus. A multiplex PCR was performed for vector backbone analysis.

TABLE 4
Summary of the molecular analysis to identify precise SSI events

| | Copy# | | | | PCR | | | |
|---|---|---|---|---|---|---|---|---|
| Event | DsRed | PMI | NPTII | FLPm | FRT1 | FRT87 | Backbone | Comments |
| 1 | 0.61 | 0.58 | null | 1.16 | + | + | + | Imprecise SSI |
| 2 | 0.96 | 1.12 | null | null | + | + | − | Precise SSI |
| 3 | 1.07 | 1.00 | null | null | + | + | − | Precise SSI |
| 4 | 0.94 | 0.98 | null | null | + | + | − | Precise SSI |
| 5 | 0.87 | 0.84 | null | null | + | + | − | Precise SSI |
| 6 | null | null | null | 0.58 | − | − | + | Imprecise SSI |
| 7 | 0.88 | 0.53 | 0.52 | 1.76 | + | − | + | Imprecise SSI |
| 8 | 1.08 | 1.06 | null | null | + | − | − | Precise SSI |
| 9 | 1.99 | 2.22 | null | null | + | + | + | Imprecise SSI |
| 10 | null | null | null | 1.22 | − | − | + | Imprecise SSI |
| 11 | 1.89 | 2.12 | null | null | + | + | + | Imprecise SSI |

The molecular analysis of the T0 events and the frequency of SSI events recovered from the two strains from multiple experiments are represented in Tables 3 and 4. No precise SSI events were recovered from the two T0 events regenerated from the LBA4404 retransformations, while four out of the nine events regenerated from the AGL1 retransformations were identified as precise SSI events. The data shows higher recovery of T0 events and precise SSI events from embryos retransformed with the Agrobacterium strain AGL1 (Table 3). Therefore, both strains have the ability to produce putative SSI events; however AGL1 produced more precise events than LBA4404.

### Example 6

### Cell Proliferation Factors

To determine the effect of cell proliferation factors on SSI event production using agrobacterium mediated delivery, transfer cassettes comprising various constructs were tested. In one experiment, Transfer Cassette S: comprising Transfer Cassette 1: RB-RS1-POI-RS2-R1-LB (PHP60577; SEQ ID NO: 12) and Transfer Cassette 2: RB-RS3-R2-CPF-RS3-LB (PHP44542 (SEQ ID NO: 15) (see Table 1 S) generated in Example 4 was delivered from a single Agrobacterium strain to retransform the target locus (gDNA-Pro-RS1-RS2-gDNA) (see Table 1) generated and described in Example 3 was compared to Transfer Cassette A: RB-RS1-POI-RS2-R1-LB (PHP60577; SEQ ID NO: 12) (see Table 1 A) delivered from a single Agrobacterium strain to retransform the target locus (gDNA-Pro-RS1-RS2-gDNA) (see Table 1).

Table 5 summarizes the transformation frequency and SSI frequency using Transfer Cassette S and Transfer Cassette A. Stable SSI events were recovered after retransformation of the target locus (gDNA-Pro-RS1-RS2-gDNA) (see Table 1) with strain LBA4404 harboring Transfer Cassette S while no SSI events were recovered after retransformation with strain LBA4404 harboring Transfer Cassette A (see Table 5). A similar trend was observed in strain AGL1 harboring Transfer Cassette S when compared to strain AGL1 harboring Transfer Cassette A (see Table 5). Significant improvement in recovery of putative SSI events was observed in experiments using Transfer Cassette S compared to Transfer Cassette A.

Precise site-specific integration events were determined using multiplex PCR assays and qPCR/PCR as described in Example 5 with certain modifications to detect the cell proliferation construct using multiplex PCR to detect the BBM gene and the Cre gene (see Table 5). A precise SSI event was identified using the following criteria:

1) a single intact copy of the polynucleotide construct (PMI and DsRed genes);

2) the absence of the target gene (NptII) at the target locus and the absence of the FLP recombinase construct;

3) the presence of the FRT1 and FRT87 junctions;

4) the absence of vector backbone; and

5) the absence of the cell proliferation factor construct genes (BBM and Cre) (see Table 5).

All other events which did not meet the above criteria were identified as imprecise SSI events.

The data also showed that precise SSI events were obtained through transient expression of the cell proliferation factors of Transfer Cassette 2: RB-RS3-R2-CPF-RS3-LB (PHP44542 (SEQ ID NO: 15) as evidenced by the absence of insertion of the cell proliferation factor genes (BBM and CRE) of the cell proliferation construct at the target locus in the precise SSI events identified (Table 6).

TABLE 5
Transformation frequency and SSI frequency
using the different Transfer Cassettes

| Vector/ | #Em- | #Events | | | Frequency | | |
|---|---|---|---|---|---|---|---|
| Strain | bryos | Callus | T0 | SSI | Callus | T0 | SSI |
| Transfer | 3376 | 13 | 9 | 4 | 0.4% | 0.27% | 0.12% |
| Cassette | | | | | | | |
| A/AGL1 | | | | | | | |
| Transfer | 3436 | 129 | 91 | 38 | 3.75% | 2.65% | 1.12% |
| Cassette | | | | | | | |
| S/AGL1 | | | | | | | |
| Transfer | 4015 | 12 | 2 | 0 | 0.3% | 0.05% | 0% |
| Cassette | | | | | | | |
| A/LBA4404 | | | | | | | |
| Transfer | 3953 | 84 | 24 | 5 | 2.13% | 0.61% | 0.13% |
| Cassette | | | | | | | |
| S/LBA4404 | | | | | | | |

TABLE 6
Summary of the molecular analysis to identify precise
SSI events retransformed with strain AGL1

| | | | | | | PCR | | | | |
|---|---|---|---|---|---|---|---|---|---|---|
| | | Copy# | | | | FRT | | ODP2/ | | |
| Event | Construct | DsRed | PMI | NptII | FLP | 1 | 87 | Cre | BB | Comment |
| 1 | Transfer | 1.02 | 1.10 | null | null | + | + | − | − | Precise SSI |
| | Cassette A | | | | | | | | | |
| 2 | Transfer | null | null | 0.87 | null | − | + | − | − | Imprecise SSI |
| | Cassette A | | | | | | | | | |
| 3 | Transfer | 1.05 | 2.12 | null | null | + | + | − | + | Imprecise SSI |
| | Cassette A | | | | | | | | | |
| 4 | Transfer | 1.00 | 0.96 | null | null | + | + | − | − | Precise SSI |
| | Cassette A | | | | | | | | | |
| 5 | Transfer | null | null | null | 1.12 | − | − | − | − | Imprecise SSI |
| | Cassette A | | | | | | | | | |
| 6 | Transfer | null | null | null | 1.00 | − | − | − | − | Imprecise SSI |
| | Cassette A | | | | | | | | | |
| 7 | Transfer | 1.08 | 0.87 | null | null | + | + | − | − | Precise SSI |
| | Cassette S | | | | | | | | | |
| 8 | Transfer | 1.99 | 2.17 | 0.96 | null | + | + | + | − | Imprecise SSI |
| | Cassette S | | | | | | | | | |
| 9 | Transfer | 0.04 | 0.04 | null | 1.00 | + | + | − | + | Imprecise SSI |
| | Cassette S | | | | | | | | | |
| 10 | Transfer | 1.08 | 1.05 | null | null | + | + | − | − | Imprecise SSI |
| | Cassette S | | | | | | | | | |
| 11 | Transfer | 1.10 | 0.93 | null | null | + | + | − | − | Imprecise SSI |
| | Cassette S | | | | | | | | | |
| 12 | Transfer | 1.40 | 0.46 | null | 1.05 | + | + | − | − | Imprecise SSI |
| | Cassette S | | | | | | | | | |
| 13 | Transfer | 1.12 | 0.96 | null | null | + | + | − | − | Imprecise SSI |
| | Cassette S | | | | | | | | | |
| 14 | Transfer | 1.08 | 0.97 | null | null | + | + | − | − | Precise SSI |
| | Cassette S | | | | | | | | | |
| 15 | Transfer | 0.95 | 0.87 | null | null | + | + | − | − | Precise SSI |
| | Cassette S | | | | | | | | | |
| 16 | Transfer | 1.01 | 1.04 | null | null | + | + | − | − | Precise SSI |
| | Cassette S | | | | | | | | | |
| 17 | Transfer | 1.00 | 0.94 | null | null | + | + | − | − | Precise SSI |
| | Cassette S | | | | | | | | | |
| 18 | Transfer | 0.91 | 0.88 | null | null | + | + | − | − | Precise SSI |
| | Cassette S | | | | | | | | | |

A second maize inbred PH184C containing the target locus (gDNA-Pro-RS1-RS2-gDNA) generated in Example 3 was tested using Agrobacterium strain AGL1 harboring Transfer Cassette S. The transformation frequency and frequency of site-specific recombination events are summarized in Table 7. These results demonstrate successful Agro SSI event generation in multiple maize inbreds.

TABLE 7
Transformation frequency and SSI frequency
in maize inbred PH184C

| | | T0 | | SSI | |
|---|---|---|---|---|---|
| Vector/Strain | #Embryos | #Events | Freq | #Events | Freq |
| Transfer Cassette | 2068 | 3 | 0.15% | 3 | 0.15% |
| S/AGL1 | | | | | |

### Example 7

### Single Transfer Cassettes

Single Transfer Cassettes, Transfer Cassette F: RB-R1-RS1-POI-RS2-RS3-R2-CPF-RS3-LB (see Table 1 F) and Transfer Cassette F1: RB-RS3-R2-CPF-RS3-RS1-POI-RS2-R1-LB (see Table 1 F1) described in Example 4 were evaluated. Transfer Cassette F and Transfer Cassette F1 were tested independently for site-specific integration (SSI) by retransforming immature embryos of maize inbred HC69 carrying the target locus (gDNA-Pro-RS1-RS2-gDNA) (see Table 1) as described in Example 3 with Agrobacterium strain AGL1 harboring the Transfer Cassettes. Transformation frequency and site-specific recombination frequency is shown in Table 8.

TABLE 8
Transformation frequency and SSI frequency in maize inbred HC69

| | | T0 | | SSI | |
|---|---|---|---|---|---|
| Construct | #Embryos | #Events | Freq | #Events | Freq |
| Transfer | 3218 | 149 | 4.63% | 39 | 1.2% |
| Cassette F | | | | | |
| Transfer | 3213 | 160 | 4.98% | 33 | 1.02% |
| Cassette F1 | | | | | |

Molecular characterization of the SSI events was performed as previously described above. Higher transformation frequencies were observed with Transfer Cassette F and Transfer Cassette F1 when compared to Transfer Cassette A and Transfer Cassette S. A significantly higher rate of T0 event recovery was also observed with Transfer Cassette F and Transfer Cassette F1 (4.6% and 5.0%, respectively). The recovery of precise SSI events was higher with Transfer Cassette F and Transfer Cassette F1 ranging from 1.0% to 1.2% compared to the precise SSI frequencies obtained with Transfer Cassette A and Transfer Cassette S.

Furthermore, Agro SSI was demonstrated with Transfer Cassette F and Transfer Cassette F1 using the Agrobacterium strain AGL1 in a second maize inbred PH184C containing a target locus (gDNA-Pro-RS1-RS2-gDNA) (see Table 1). The data summarizing the transformation frequency and SSI frequency is presented in Table 9.

TABLE 9
Transformation frequency and SSI frequency with Transfer
Cassette F and F1 in inbred line PH184C.

| | | T0 | | SSI | |
|---|---|---|---|---|---|
| Vector | #Embryos | #Events | Freq | #Events | Freq |
| Transfer | 2055 | 5 | 0.24% | 4 | 0.2% |
| Cassette F | | | | | |
| Transfer | 2055 | 13 | 0.63% | 1 | 0.05% |
| Cassette F1 | | | | | |

The above examples demonstrated successful validation of Agro SSI in multiple corn inbred lines retransformed with different Transfer Cassettes.

### Example 8

### Agro SSI Using Multiple FRT Pairs

Multiple recombination sites (FRT1 (SEQ ID NO.7), FRT6 (SEQ ID NO: 34), FRT12 (SEQ ID NO: 9), and FRT87 (SEQ ID NO.8) and combinations were tested to determine their effect on Agro SSI. Agro SSI was tested in multiple events containing the target locus (gDNA-Pro-RS1-RS2-gDNA) (see Table 1). Target loci were generated in maize inbred line HC69 as described in Example 3. HC69 maize inbred lines comprising the following non-identical recombination sites were generated:
1) FRT1 (RS1) and FRT6 (RS2);
2) FRT1 (RS1) and FRT12 (RS2); and,
3) FRT1 (RS2) and FRT87 (RS2).

Three different configurations of Transfer Cassette F (see Table 1 F) were generated:
1) RB-R1-RS1(FRT1)-POI-RS2(FRT6)-RS3-R2-CPF-RS3-LB
2) RB-R1-RS1(FRT1)-POI-RS2(FRT12)-RS3-R2-CPF-RS3-LB
3) RB-R1-RS1(FRT1)-POI-RS2(FRT87)-RS3-R2-CPF-RS3-LB
   In each configuration of Transfer Cassette F: RB-R1-RS1-POI-RS2-RS3-R2-CPF-RS3-LB the restriction site 1 (RS1) flanking the polynucleotide construct (POI) was (FRT1) and restriction site 2 (RS2) varied and was either FRT6, FRT12, or FRT87. All other components of these configurations of Transfer Cassette F were the same using components described above. Agrobacterium strain AGL1 harboring Transfer Cassette F comprising the different combination of the recombination sites (FRT1/6; FRT1/12 and FRT1/87) was used for determining SSI frequency in maize inbred line HC69 with the different target loci consisting of FRT1/6, FRT1/12 and FRT1/87.

Table 9 summarizes transformation frequency and site-specific recombination frequency results with different Transfer Cassettes containing the different combinations of recombination sites FRT1/6, FRT1/12, and FRT1/87. The Transfer Cassettes were introduced to the HC69 maize inbred target lines each having a corresponding recombination site pair at the target locus.

TABLE 9
Transformation frequency and SSI frequency in target loci
with different pairs of recombination sites in inbred HC69

| | |
|---|---|
| Target | |
| loci | SSI |

| FRT Pair | #Embryos | T0 Freq | #Events | Freq |
|---|---|---|---|---|
| 1/6 | 462 | 22.5% | 32 | 6.9% |
| 1/12 | 676 | 19.1% | 45 | 6.7% |
| 1/87 | 3218 | 4.6% | 39 | 1.2% |

Higher T0 transformation frequencies were observed in target loci consisting of the FRT recombination sites FRT1/6 and FRT1/12 (22.5% and 19%, respectively) as compared to the target loci containing recombination sites FRT 1/87. The increase in the T0 level transformation frequency ranged anywhere from 2.7-fold to 3.2-fold in target loci consisting of the recombination sites FRT1/6 and FRT1/12 as compared to the target loci consisting of the recombination sites FRT 1/87 (Table 9). Similarly, the precise SSI frequency in target loci consisting of the recombination sites FRT 1/6 and FRT1/12 was about 3.5-fold higher than in target loci consisting of the recombination sites FRT 1/87. The above data documents recovery of precise SSI events using multiple non-identical recombination sites.

### Example 9

### Agro SSI Using a SU Inducible Germplasm in Maize Inbred PHHSG

Agro SSI was further demonstrated in the transformation of recalcitrant maize inbred PHHSG. In this experiment, multiple lines containing the target locus (gDNA-Pro-RS1-RS2-gDNA) (see Table 1) were generated in maize inbred PHHSG using Agrobacterium-mediated transformation of immature embryos as described in Example 3 with the following modifications: Maize inbred PHHSG was transformed with a different Transfer Cassette, namely: RB-ZmUbiPro-FRT1-DsRED::PinII+ZmUbiPro::GAT4602:PinII-FRT87-LB (PHP41877; SEQ ID NO: 18) to generate lines that contained the target locus (gDNA-Pro-RS1-RS2-gDNA) (see Table 1).

Agrobacterium strain AGL1 harboring Transfer Cassette A: RB-RS1-POI-RS2-R1-LB (see Table 1 A) comprising of the following polynucleotide sequence FRT1-NptII::PinII+ZmUbiPro::AmCyan (CFP)::PinII-FRT87 (PHP60546 (SEQ ID NO: 19)), or harboring two transfer cassettes (see Table 1S). Specifically, Transfer Cassette S comprising Transfer Cassette 1: RB-RS1-POI-RS2-R1-LB comprising the polynucleotide sequence RB-FRT1-NptII::PinII+ZmUbiPro::AmCyan::PinII-FRT87-LB (PHP60546 (SEQ ID NO: 19)) and Transfer Cassette 2: RB-RS3-R2-CPF-RS3-LB (PHP44542 (SEQ ID NO: 15)) were used for determining SSI event frequency. The above transfer cassettes failed to produce SSI events.

In a second experiment, lines with an inducible cell proliferation factor construct were generated in maize inbred PHH5G by transforming with a different Transfer Cassette namely RB-NosProTetOp1-ZmUbiPro::WUS::PinII+ZmUbiPro3XTetOp::ZmODP2::PinII:GZ-W64A+ZmUbiPro::ESR::PinII+SbALSPro::ZmALS::PinII-LB (PHP60850 (SEQ ID NO: 20)), and lines were selected (ESR is an ethametsulfuron responsive repressor that interacts with tetracycline operators (TetOp) (see, e.g., U.S. Pat. No. 8,257,956, the contents of which is herein incorporated by reference in its entirety)). The inducible lines with the cell proliferation factors were then crossed to the lines with the target locus RB-ZmUbiPro-FRT1-DsRED::PinII+ZmUbiPro::GAT4602:PinII-FRT87-LB (PHP41877; SEQ ID NO: 18) generated above. Homozygous lines consisting of the target locus RB-ZmUbiPro-FRT1-DsRED::PinII+ZmUbiPro::GAT4602:PinII-FRT87 (PHP41877; SEQ ID NO: 18) and the inducible cell proliferation factor RB-NosProTetOp1-ZmUbiPro::WUS::PinII+ZmUbiPro3XTetOp::ZmODP2::PinII:GZ-W64A+ZmUbiPro::ESR::PinII+SbALSPro::ZmALS::PinII-LB (PHP60850 (SEQ ID NO: 20)) were identified using event specific PCR primers. The embryos derived from the above target locus with the inducible cell proliferation factor were then retransformed with Agrobacterium strain AGL1 containing two transfer cassettes as in Table 1 S; Transfer Cassette 1: RB-RS1-POI-RS2-R1-LB comprising the polynucleotide sequence RB-FRT1-NptII::PinII+ZmUbiPro::AmCyan::PinII-FRT87-LB (PHP60546 (SEQ ID NO: 19)) and Transfer Cassette 2: RB-RS3-R2-CPF-RS3-LB (PHP44542 (SEQ ID NO: 15)) to determine SSI frequency and the recovery of precise SSI events (Table 10). Table 10 summarizes transformation frequency and site-specific recombination frequency results in a different genotypic target loci (maize inbred PHH5G) using the two Transfer Cassette design as illustrated in Table 1 S. This data demonstrates successful use of maize inbred PHH5G containing the cell proliferation factor construct and the target loci described above for generating precise SSI events.

TABLE 10

| | | | SSI | |
|---|---|---|---|---|
| Target Loci | Transfer Cassettes | #Embryos | #Events | Freq |
| PHP41877 + | PHP60546 + PHP44542 | 1133 | 5 | 0.44% |
| PHP60850 | | | | |
| PHP41877 | PHP60546 + PHP44542 | 1177 | 0 | 0% |

### Example 10

### Modulating Cell Proliferation Factor Expression

Experiments were performed to determine if modulating the expression of the cell proliferation factors contained in the cell proliferation factor construct (CPF) resulted in an increased SSI frequency. Transfer Cassette F: RB-R1-RS1-POI-RS2-RS3-R2-CPF-RS3-LB was used with different promoters to drive the cell proliferation factor genes in the cell proliferation factor construct (CFP) thereby modulating expression of the cell proliferation factor genes WUS and ODP2 (BBM) present either alone or in combination in the cell proliferation factor construct (CFP). The following cell proliferation factor constructs (CFP) were generated with different promoters driving expression of the cell proliferation factor genes:
1) PHP71518 (SEQ ID NO: 21): OsActinPro::WUS::PINII+ZmUbiPro::BBM::PINII;
2) PHP71298 (SEQ ID NO: 22): CaMV35SPro::WUS::PINII+ZmUbiPro::BBM::PINII;
3) PHP71867 (SEQ ID NO: 23): ZmUbiPro::WUS::PINII+ZmUbiPro::BBM::PINII;
4) PHP71336 (SEQ ID NO: 24): NosPro::WUS::PINII+35S-Enhancer-ZmUbiPro::BBM::PINII;
5) PHP72235 (SEQ ID NO: 25): ZmUbiPro::WUS::PINII+35S enhancer::ZmUbiPro::BBM::PINII;
6) PHP73860 (SEQ ID NO: 26): BSVPro::WUS::PINII+ZmUbiPro::BBM::PINII;
7) PHP76975 (SEQ ID NO: 27): ZmUbiPro::WUS::PINII; and
8) PHP77277 (SEQ ID NO: 28): BSVPro::WUS::PINII.
   All other components in the Transfer Cassette F configurations were the same and as previously described.

Agrobacterium strain AGL1 harboring Transfer Cassette F: RB-R1-RS1-POI-RS2-RS3-R2-CPF-RS3-LB with the various promoter combinations driving expression of the cell proliferation factors in the cell proliferation construct were tested independently for site-specific integration (SSI) by transforming maize cultivars, HC69 and PH184C carrying the target locus (gDNA-Pro-RS1-RS2-gDNA) (see Table 1). Stable expression of fluorescent protein DsRed was used to monitor the effect of the cell proliferation factors on cell division and SSI event recovery. Cell proliferation factor constructs containing the ZmUbiPro-35S Enhancer:: or the OsActinPro:: driving expression of the WUS cell proliferation factor stimulated cell division to a much greater extent in the two maize inbred lines tested, as compared to the cell proliferation construct with the NosPro driving expression of the WUS cell proliferation factor.

The data from these experiments using maize inbred lines PH184C and HC69 are summarized in Table 11 and Table 12, respectively. We demonstrated that different promoters driving expression of the cell proliferation factors in the cell proliferation construct enabled Agro SSI in multiple genotypes. The data also showed Agro SSI frequency varied with the choice of the promoter used for driving expression of the cell proliferation factors.

TABLE 11
Transformation Frequency and SSI Frequency with Transfer Cassettes
comprising different promoters driving expression of the cell
proliferation factor genes in maize inbred PH184C

| | | Transfer | | | | |
|---|---|---|---|---|---|---|
| | | Cas- | | Frequency | | SSI |
| Promoter | | settte | #Em- | T0 | | Conver- |
| WUS | BBM | PHP | bryos | Event | SSI | sion |
| CaMV35S | ZmUbi | 71298 | 1551 | 0.39% | 0.13% | 33% |
| Nos | 35SEnhZmUbi | 71336 | 1551 | 0.26% | 0.06% | 25% |
| OsAct-Int | ZmUbi | 71518 | 1477 | 1.29% | 0.74% | 52% |
| ZmUbi | ZmUbi | 71867 | 1543 | 0.84% | 0.52% | 62% |
| ZmUbi | 35SEnhZmUbi | 72235 | 1564 | 0.95% | 0.58% | 60% |

TABLE 12
Transformation Frequency and SSI Frequency with Transfer Cassettes
comprising different promoters driving expression of the cell
proliferation factor genes in maize inbred HC69

| | | Transfer | | Frequency | | | |
|---|---|---|---|---|---|---|---|
| Promoter | | Cassettte | | Callus | T0 | | SSI |
| WUS | BBM | PHP | #Embryos | Event | Event | SSI | Conversion |
| CaMV35S | ZmUbi | 71298 | 1397 | 3.8% | 1.15% | 0.4% | 37.5% |
| CaMV35S | ZmUbi | 71298 | 1398 | 4.1% | 0.72% | 0.1% | 20.0% |
| Nos | 35SEnhZmUbi | 71336 | 958 | 3.2% | 1.46% | 0.5% | 35.7% |
| OsAct-Int | ZmUbi | 71518 | 845 | 11.2% | 5.92% | 1.9% | 32.0% |
| ZmUbi | ZmUbi | 71867 | 886 | 7.0% | 5.42% | 1.6% | 29.2% |
| ZmUbi | 35SEnhZmUbi | 72235 | 818 | 1.3% | 1.10% | 0.4% | 33.3% |
| BSV | ZmUbi | 73860 | 814 | 4.4% | 3.32% | 1.1% | 33.3% |

### Example 11

### Single FRT Site Target Locus For Agro SSI

Agro SSI efficiency using a single FRT1 site (SEQ ID NO: 7) was also determined in a separate experiment. A target locus comprising a promoter trap operably linked to the single recombination site (gDNA-Pro-RS1-gDNA) was created in maize inbred PHWWE using Agrobacterium-mediated immature embryo transformation. Hemizygous embryos derived from the maize inbred PHWWE lines with the target locus (gDNA-Pro-RS1-gDNA) were identified and were retransformed with a single transfer cassette, Transfer cassette F; (PHP62258 (SEQ ID NO: 14)) see Table 1 F. A total of 46 T0 plants were generated from 24 events. Molecular analysis of the T0 plants was carried out as described in Example 5. None of the recovered T0 plants were identified as SSI events, showing that target loci with a single recombination site are not good targets for efficient Agrobacterium-mediated SSI event generation in maize. The data is summarized in Table 13.

TABLE 13

| | | PCR | | |
|---|---|---|---|---|
| Plant ID | Event ID | FRT87 | UbiPSB1 | PMI |
| 1 | 1 | − | − | − |
| 2 | 1 | − | − | − |
| 3 | 1 | − | − | − |
| 4 | 2 | − | − | − |
| 5 | 2 | − | − | − |
| 6 | 2 | − | − | − |
| 7 | 3 | − | + | − |
| 8 | 3 | − | + | − |
| 9 | 3 | − | + | − |
| 10 | 4 | − | + | − |
| 11 | 4 | − | + | − |
| 12 | 4 | − | + | − |
| 13 | 5 | − | − | − |
| 14 | 5 | − | − | − |
| 15 | 5 | − | − | − |
| 16 | 6 | − | + | − |
| 17 | 7 | − | + | − |
| 18 | 8 | − | + | − |
| 19 | 9 | − | + | − |
| 20 | 10 | − | + | − |
| 21 | 11 | − | + | − |
| 22 | 12 | − | + | − |
| 23 | 12 | − | + | − |
| 24 | 13 | − | + | − |
| 25 | 13 | − | + | − |
| 26 | 13 | − | + | − |
| 27 | 14 | − | + | − |
| 28 | 14 | − | + | − |

### Example 12

### Improved Recovery of Site-Specific Recombination Events

Agrobacterium mediated SSI was used with Transfer Cassettes containing cell proliferation factor constructs comprising AXIG1 PRO::WUS2+PLTP PRO::ODP2. Two Transfer Cassettes were used to retransform PHC184 containing target locus target locus (gDNA-Pro-RS1-RS2-gDNA) (see Table 1). The first Transfer Cassette (RB-R1-RS1-POI-CPF-RS2-LB): (RB-UBI PRO:UBI1ZM INTRON::MO-FLP::PINII TERM+CaMV35S TERM+FRT1:PMI::PINII TERM+ZM-AXIG1 PRO::ZM-WUS2::IN2-1 TERM+ZM-PLTP PRO::ZM-ODP2::OS-T28 TERM+UBI PRO::UBI1ZM INTRON::DsRED: FRT87-LB) (RV003866; (SEQ ID NO: 37)) and the second Transfer Cassette (RB-CPF-R1-RS1-POI-RS2-LB): (RB-+ZM-AXIG1 PRO::ZM-WUS2::IN2-1 TERM+ZM-PLTP PRO::ZM-ODP2::OS-T28 TERM UBI PRO:UBI1ZM INTRON::MO-FLP::PINII TERM+CaMV35S TERM+FRT1:PMI::PINII TERM+UBI PRO::UBI1ZM INTRON::DsRED: FRT87-LB) (RV004886; (SEQ ID NO: 38)) were delivered in two separate experiments. The first Transfer Cassette contained the polynucleotide construct (PMI/DsRED) and the cell proliferation factor construct (WUS2, ODP2) within the flanking FRT1 and FRT87 non-identical recombination sites and the second Transfer Cassette contained only the polynucleotide construct (PMI/DsRED) within the flanking FRT1 and FRT87 non-identical recombination sties. Each of the Transfer Cassettes was delivered via Agro-mediated transformation, as described in U.S. Provisional Appln. No. 62/248,578 incorporated herein by reference in its entirety and in U.S. Provisional Appln. No. 62/271,230 incorporated herein by reference in its entirety, into target lines containing the target locus (gDNA-Pro-RS1-RS2-gDNA) (see Table 1). Precise SSI events were identified using a multiplex PCR assay as described in Example 5. The data is summarized in Table 14. The use of a cell proliferation factor construct containing AXIG1 PRO::WUS2+PLTP PRO::ODP2 shortened the entire SSI process by several weeks (at least 3-4 weeks), compared to the normal transformation methods used for generating SSI events.

TABLE 14
Recovery of Precise SSI events in Agro SSI experiments
using WUS2 and ODP2 expression.

| | # embryos | # T0 | | |
|---|---|---|---|---|
| Vector | infected | plants | # SSI | % SSI |
| RV003866 | 859 | 42 | 6 | 0.7 |
| RV004886 | 836 | 10 | 1 | 0.1 |

### Example 13

### Target Lines comprising the Cell Proliferation Factor and Recombinase

Transfer Cassettes containing a cell proliferation construct and/or a recombinase construct may be used to create target lines having one or more of these constructs flanked by non-identical recombination sites as part of the target locus. This experimental strategy may improve recovery of precise SSI events, as well as simplify the Transfer Cassette design for retransformation. Site-specific integration at the new target loci will require delivery of only a Transfer Cassette, with no recombinase construct and/or cell proliferation factor construct.

Transfer Cassettes containing a cell proliferation construct and/or a recombinase construct may be used to create target lines having one or more of these constructs outside non-identical recombination sites and not part of the target locus. This experimental strategy may improve recovery of precise SSI events, as well as simplify the Transfer Cassette design for retransformation. Site-specific integration at the new target loci will require delivery of only a Transfer Cassette, with no recombinase construct and/or cell proliferation factor construct. The cell proliferation factor construct and/or the recombinase construct could be removed from the SSI event by crossing and segregation.

Various transfer cassette elements, configurations, target loci, and recombination products have been illustrated herein, and many modifications and other examples are expressly taught or disclosed to a person of skill in the art. Various specific T-DNAs, transfer cassettes, and operably linked construct elements have been provided, and are summarized in Table 15 below. Many modifications, alternate configurations, alternate elements, and optional elements are expressly taught or disclosed to a person of skill in the art, and further designs are within the skill in the art.

TABLE 15

| SEQ# | Name |
|---|---|
| 1 | PHP64484 |
| 2 | ZmUbiPro |
| 3 | DsRed |
| 4 | DsRed |
| 5 | YFP |
| 6 | YFP |
| 7 | FRT1 |
| 8 | FRT87 |
| 9 | FRT12 |
| 10 | FLP exon1 |
| 11 | FLP exon2 |
| 12 | PHP60577 |
| 13 | PHP62558 |
| 14 | PHP62258 |
| 15 | PHP44542 |
| 16 | Spectinomycin |
| 17 | Kanamycin |
| 18 | PHP41877 |
| 19 | PHP60546 |
| 20 | PHP60850 |
| 21 | PHP71518 |
| 22 | PHP71298 |
| 23 | PHP71867 |
| 24 | PHP71336 |
| 25 | PHP72235 |
| 26 | PHP73860 |
| 27 | PHP76975 |
| 28 | PHP77277 |
| 29 | CRE exon 1 |
| 30 | CRE exon 2 |
| 31 | LOXP |
| 32 | NosPro::WUS::PINII |
| 33 | ZmUbiPro:BBM:PINII |
| 34 | FRT6 |
| 35 | AXIG1 |
| | PRO::WUS2:IN2-1 |
| 36 | PLTP |
| | PRO::ODP2::OS-T28 |
| 37 | RV003866 |
| 38 | RV004886 |
| 39 | NptII |
| 40 | PINII TERM |
| 41 | AmCyan (CFP) |
| 42 | PMI |
| 43 | Rab17PRO |

As used herein the singular forms “a”, “an”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this disclosure belongs unless clearly indicated otherwise.

All patents, publications and patent applications mentioned in the specification are indicative of the level of those skilled in the art to which this disclosure pertains. All patents, publications and patent applications are herein incorporated by reference in its entirety to the same extent as if each individual patent, publication or patent application was specifically and individually indicated to be incorporated by reference in its entirety.

Although the foregoing disclosure has been described in some detail by way of illustration and example for purposes of clarity of understanding, certain changes and modifications may be practiced within the scope of the appended claims.

That which is claimed:

1. An agrobacterium-mediated transformation method for targeting the insertion of a polynucleotide of interest to a genomic target locus in a monocot plant cell, the method comprising:
introducing into the monocot plant cell a transfer cassette, the transfer cassette flanked by a right border and a left border and comprising between the right border and the left border, in operable linkage:
a) a polynucleotide construct comprising the polynucleotide of interest, the polynucleotide construct flanked by a first recombination site and a second non-identical recombination site; and
b) a recombinase construct encoding a recombinase;
wherein the monocot plant cell comprises the genomic target locus comprising the first recombination site and the second non-identical recombination site, whereby the recombinase recognizes and implements recombination at the first and the second non-identical recombination sites such that the polynucleotide construct is inserted at the target locus.

2. An agrobacterium-mediated transformation method for targeting the insertion of a polynucleotide of interest to a genomic target locus in a plant cell, the method comprising:
introducing into the plant cell a transfer cassette, the transfer cassette flanked by a right border and a left border and comprising between the right border and the left border, in operable linkage:
a) a polynucleotide construct comprising the polynucleotide of interest, the polynucleotide construct flanked by a first recombination site and a second non-identical recombination site;
b) a cell proliferation factor construct encoding a cell proliferation factor; and
c) a recombinase construct encoding a recombinase;
wherein the plant cell comprises the genomic target locus comprising the first recombination site and the second non-identical recombination site; whereby the recombinase recognizes and implements recombination at the first recombination site and the second non-identical recombination site such that the polynucleotide construct is inserted at the target locus.

3. An agrobacterium-mediated transformation method for targeting the insertion of a polynucleotide of interest to a genomic target locus in a monocot plant cell, the method comprising:
introducing into the monocot plant cell a transfer cassette, the transfer cassette flanked by a right border and a left border and comprising between the right border and the left border, in operable linkage:
a) a polynucleotide construct comprising the polynucleotide of interest, the polynucleotide construct flanked by a first recombination site and a second non-identical recombination site; and
b) a recombinase construct encoding a recombinase, wherein the recombinase construct is flanked by a third recombination site and a fourth identical recombination site, wherein the third recombination site and the fourth identical recombination site are non-identical to the first recombination site and to the second non-identical recombination site;
wherein the plant cell comprises the genomic target locus comprising the first recombination site and the second non-identical recombination site; whereby the recombinase recognizes and implements recombination at the first recombination site and the second non-identical recombination site such that the polynucleotide construct is inserted at the target locus.

4. An agrobacterium-mediated transformation method for targeting the insertion of a polynucleotide of interest to a genomic target locus in a plant cell, the method comprising:
introducing into the plant cell a transfer cassette, the transfer cassette flanked by a right border and a left border and comprising between the right border and the left border, in operable linkage:
a) a polynucleotide construct comprising the polynucleotide of interest, the polynucleotide construct flanked by a first recombination site and a second non-identical recombination site; and,
b) a cell proliferation factor construct encoding a cell proliferation factor and a recombinase construct encoding a recombinase, the cell proliferation factor construct and the recombinase construct are flanked by a third recombination site and a fourth identical recombination site, wherein the third recombination site and the fourth identical recombination site are non-identical to the first recombination site and the second non-identical recombination site;
wherein the plant cell comprises the genomic target locus comprising the first recombination site and the second non-identical recombination site; whereby the recombinase recognizes and implements recombination at the first recombination site and the second non-identical recombination site such that the polynucleotide construct is inserted at the target locus.

5. An agrobacterium-mediated transformation method for targeting the insertion of a polynucleotide of interest to a genomic target locus in a plant cell, the method comprising:
introducing into the plant cell a transfer cassette, the transfer cassette flanked by a right border and a left border and comprising between the right border and the left border, in operable linkage:
a) a polynucleotide construct comprising the polynucleotide of interest, the polynucleotide construct flanked by a first recombination site and a second non-identical recombination site;
b) a first recombinase construct encoding a first recombinase; and,
c) a cell proliferation factor construct encoding a cell proliferation factor, and a second recombinase construct encoding a second recombinase, the cell proliferation factor construct and the second recombinase construct flanked by a third recombination site and a fourth identical recombination site, wherein the third recombination site and the fourth identical recombination site are non-identical to the first recombination site and the second non-identical recombination site;
wherein the plant cell comprises the genomic target locus comprising the first recombination site and the second non-identical recombination site; whereby the first recombinase recognizes and implements recombination at the first recombination site and the second non-identical recombination site such the polynucleotide construct is inserted at the target locus, and whereby the second recombinase recognizes and implements recombination at the third recombination site and the fourth identical recombination site thereby excising the cell proliferation factor construct and the second recombinase construct.

6. An agrobacterium-mediated transformation method for targeting the insertion of a polynucleotide of interest to a genomic target locus in a plant cell, the method comprising:
introducing into the plant cell a transfer cassette, the transfer cassette flanked by a right border and a left border and comprising between the right border and the left border, in operable linkage:
a) a first recombination site adjacent to the right border and a second recombination site adjacent to the left border, wherein the first recombination site is non-identical to the second recombination site;
b) a polynucleotide construct comprising the polynucleotide of interest, the polynucleotide construct flanked by the first recombination site and a third recombination site, wherein the third recombination site is non-identical to the first recombination site and the second non-identical recombination site; and,
c) a cell proliferation factor construct encoding a cell proliferation factor and a recombinase construct encoding a recombinase, wherein the cell proliferation factor construct and the recombinase construct are flanked by the third recombination site and a fourth recombination site, wherein the fourth recombination site is identical to the third recombination site:
wherein the plant cell comprises the genomic target locus comprising the first recombination site and the second non-identical recombination site; whereby the recombinase recognizes and implements recombination at the first recombination site and the second non-identical recombination site such that the polynucleotide construct is inserted at the target locus, and whereby the recombinase recognizes and implements recombination at the third recombination site and the fourth identical recombination site thereby excising the cell proliferation factor construct and the recombinase construct.

7. An agrobacterium-mediated transformation method for targeting the insertion of a polynucleotide of interest to a genomic target locus in a plant cell, the method comprising:
introducing into the plant cell a transfer cassette, the transfer cassette flanked by a right border and a left border and comprising between the right border and the left border, in operable linkage:
a) a first recombination site adjacent to the right border and a second recombination site adjacent to the left border, wherein the first recombination site is non-identical to the second recombination site;
b) a polynucleotide construct comprising the polynucleotide of interest, the polynucleotide construct flanked by the first recombination site and a third recombination site, wherein the third recombination site is non-identical to the first recombination site and the second non-identical recombination site;
c) a cell proliferation factor construct encoding a cell proliferation factor, a first recombinase construct encoding a first recombinase, and a second recombinase construct encoding a second recombinase, wherein the cell proliferation factor construct, the first recombinase construct, and the second recombinase construct are flanked by the third recombination site and a fourth recombination site, wherein the fourth recombination site is identical to the third recombination site;
wherein the plant cell comprises the genomic target locus comprising the first recombination site and the second non-identical recombination site; whereby the first recombinase recognizes and implements recombination at the first recombination site and the second non-identical recombination site and the second recombinase recognizes and implements recombination at the third recombination site and the fourth identical recombination site such that the polynucleotide construct comprising the third recombination site is inserted at the target locus.

8. An agrobacterium-mediated transformation method for targeting the insertion of a polynucleotide of interest to a genomic target locus in a plant cell, the method comprising:
introducing into the plant cell a transfer cassette, the transfer cassette flanked by a right border and a left border and comprising between the right border and the left border, in operable linkage:
a) a first recombination site adjacent to the right border and a second recombination site adjacent to the left border, wherein the first recombination site is non-identical to the second recombination site;
b) a polynucleotide construct comprising the polynucleotide of interest, the polynucleotide construct flanked by a third recombination site and a fourth non-identical recombination site, wherein the third recombination site and the fourth non-identical recombination sites are non-identical to the first recombination site, the second non-identical recombination site, and to each other;
c) a cell proliferation factor construct encoding a cell proliferation factor, wherein the cell proliferation factor construct is flanked by the first recombination site and a fifth recombination site, wherein the fifth recombination site is identical to the first recombination site;
d) a first recombinase construct encoding a first recombinase, wherein the first recombinase construct is flanked by the third non-identical recombination site and the fifth recombination site; and
e) a second recombinase construct encoding a second recombinase, wherein the second recombinase is different from the first recombinase, the second recombinase construct is flanked by the fourth recombination site and a sixth recombination site, wherein the sixth recombination site is identical to the fourth recombination site;
wherein the plant cell comprises the genomic target locus comprising the second non-identical recombination site and the third non-identical recombination site; whereby the second recombinase recognizes and implements recombination at the second non-identical recombination site, the third non-identical recombination site, the fourth non-identical recombination site, and the sixth recombination site, and the first recombinase recognizes and implements recombination at the first recombination site and the fifth identical recombination site such that after recombination the target locus comprises the third non-identical recombination site, the polynucleotide construct, the fourth non-identical recombination site, and the second non-identical recombination site all in operable linkage.

9. An agrobacterium-mediated transformation method for targeting the insertion of a polynucleotide of interest to a genomic target locus in a plant cell, the method comprising:
introducing into the plant cell a transfer cassette, the transfer cassette flanked by a right border and a left border and comprising between the right border and the left border, in operable linkage:
a) a first recombination site adjacent to the right border and a second recombination site adjacent to the left border, wherein the first recombination site is non-identical to the second recombination site;
b) a polynucleotide construct comprising the polynucleotide of interest, the polynucleotide construct flanked by a third recombination site and a fourth non-identical recombination site;
c) a cell proliferation factor construct encoding a cell proliferation factor and a first recombinase construct encoding a first recombinase, wherein the cell proliferation factor construct and the first recombinase construct are flanked by the first recombination site and a fifth recombination site, wherein the fifth recombination site is identical to the first recombination site; and
d) a second recombinase construct encoding a second recombinase, wherein the second recombinase is different than the first recombinase, wherein the second recombinase construct is flanked by the fourth recombination site and a sixth recombination site, wherein the sixth recombination site is identical to the fourth recombination site;
wherein the plant cell comprises the genomic target locus comprising the third recombination site and the second non-identical recombination site; whereby the second recombinase recognizes and implements recombination at the second non-identical recombination site and the third recombination site, the first recombinase recognizes and implements recombination at the first recombination site and the fifth identical recombination site, and either the first recombinase or the second recombinase recognizes and implements recombination at the fourth recombination site and the sixth recombination site such that after recombination the target locus comprises the third recombination site, the polynucleotide construct, the fourth non-identical recombination site, and the second non-identical recombination site all in operable linkage.

10. An agrobacterium-mediated transformation method for targeting the insertion of a polynucleotide of interest to a genomic target locus in a monocot plant cell, said method comprising:
introducing into the monocot plant cell a first transfer cassette and a second transfer cassette, each of the first transfer cassette and the second transfer cassette flanked by a right border and a left border and comprising between each right border and each left border, in operable linkage:
a) the first transfer cassette comprising a polynucleotide construct comprising the polynucleotide of interest, wherein the polynucleotide construct is flanked by a first recombination site and a second non-identical recombination site; and,
b) the second transfer cassette comprising a recombinase construct, the recombinase construct encoding a recombinase;
wherein the monocot plant cell comprises the genomic target locus comprising the first recombination site and the second non-identical recombination site, whereby the recombinase recognizes and implements recombination at the first recombination site and the second non-identical recombination site such that the polynucleotide construct is inserted at the target locus.

11. An agrobacterium-mediated transformation method for targeting the insertion of a polynucleotide of interest to a genomic target locus in a plant cell, said method comprising:
introducing into the plant cell a first transfer cassette and a second transfer cassette, each of the first transfer cassette and the second transfer cassette flanked by a right border and a left border and comprising between each right border and each left border, in operable linkage:
a) the first transfer cassette comprising a polynucleotide construct comprising the polynucleotide of interest, wherein the polynucleotide construct is flanked by a first recombination site and a second non-identical recombination site; and,
b) the second transfer cassette comprising a cell proliferation factor construct and a recombinase construct, the cell proliferation factor construct encoding a cell proliferation factor, and the recombinase construct encoding a recombinase;
wherein the plant cell comprises the genomic target locus comprising the first recombination site and the second non-identical recombination site; whereby the recombinase recognizes and implements recombination at the first recombination site and the second non-identical recombination site such that the polynucleotide construct is inserted at the target locus.

12. An agrobacterium-mediated transformation method for targeting the insertion of a polynucleotide of interest to a genomic target locus in a plant cell, said method comprising:
introducing into the plant cell a first transfer cassette and a second transfer cassette, each of the first transfer cassette and the second transfer cassette flanked by a right border and a left border and comprising between each right border and each left border, in operable linkage:
a) the first transfer cassette comprising a polynucleotide construct and a recombinase construct, wherein the polynucleotide construct comprises the polynucleotide of interest and is flanked by a first recombination site and a second non-identical recombination site, and wherein the recombinase construct encodes a recombinase; and,
b) the second transfer cassette comprising a cell proliferation factor construct, the cell proliferation factor construct encoding a cell proliferation factor;
wherein the plant cell comprises the genomic target locus comprising the first recombination site and the second non-identical recombination site, whereby the recombinase recognizes and implements recombination at the first recombination site and the second non-identical recombination site such that the polynucleotide construct is inserted at the target locus.

13. An agrobacterium mediated transformation method for targeting the insertion of a polynucleotide of interest to a genomic target locus in a plant cell, said method comprising:
introducing into the plant cell a first transfer cassette and a second transfer cassette, each of the first transfer cassette and the second transfer cassette flanked by a right border and a left border and comprising between each right border and each left border, in operable linkage:
a) the first transfer cassette comprising a polynucleotide construct comprising the polynucleotide of interest and flanked by a first recombination site and a second non-identical recombination site; and,
b) the second transfer cassette comprising a cell proliferation factor construct encoding a cell proliferation factor and a recombinase construct encoding a recombinase, wherein the cell proliferation factor construct is flanked by a third recombination site and a fourth identical recombination site, wherein the third recombination site and the fourth identical recombination site are non-identical to the first recombination site and the second non-identical recombination site;
wherein the plant cell comprises the genomic target locus comprising the first recombination site and the second non-identical recombination site; whereby the recombinase recognizes and implements recombination at the first recombination site and the second non-identical recombination site such that the polynucleotide construct is inserted at the target locus.

14. An agrobacterium mediated transformation method for targeting the insertion of a polynucleotide of interest to a genomic target locus in a plant cell, said method comprising:
introducing into the plant cell a first transfer cassette and a second transfer cassette, each of the first transfer cassette and the second transfer cassette flanked by a right border and a left border and comprising between each right border and each left border, in operable linkage:
a) the first transfer cassette comprising a polynucleotide construct comprising the polynucleotide of interest flanked by a first recombination site and a second non-identical recombination site; and
b) the second transfer cassette comprising a cell proliferation factor construct encoding a cell proliferation factor and a recombinase construct encoding a recombinase, wherein the cell proliferation factor construct and the recombinase construct are flanked by a third recombination site and a fourth identical recombination site, wherein the third recombination site and the fourth identical recombination site are non-identical to the first recombination site and the second non-identical recombination site;
wherein the plant cell comprises the genomic target locus comprising the first recombination site and the second non-identical recombination site, whereby the recombinase recognizes and implements recombination at the first recombination site and the second non-identical recombination site such that the polynucleotide construct is inserted at the target locus.

15. An agrobacterium mediated transformation method for targeting the insertion of a polynucleotide of interest to a genomic target locus in a plant cell, said method comprising:
a) introducing into the plant cell a first transfer cassette comprising, in operable linkage,
i) a polynucleotide construct comprising the polynucleotide of interest, wherein the polynucleotide construct is flanked by a first recombination site and a second non-identical recombination site; and
ii) a first recombinase construct encoding a first recombinase; and,
b) introducing into the plant cell a second transfer cassette comprising, in operable linkage;
i) a cell proliferation factor construct encoding a cell proliferation factor and flanked by a third recombination site and a fourth identical recombination site, wherein the third recombination site and the fourth identical recombination site are non-identical to the first recombination site and the second non-identical recombination site; and
ii) a second recombinase construct encoding a second recombinase, wherein the second recombinase is different than the first recombinase;
wherein the plant cell comprises the genomic target locus comprising the first recombination site and the second non-identical recombination site, whereby the first recombinase recognizes and implements recombination at the first recombination site and the second non-identical recombination site such that the polynucleotide construct is inserted at the target locus.

16. An agrobacterium mediated transformation method for targeting the insertion of a polynucleotide of interest to a genomic target locus in a plant cell, said method comprising:
a) introducing into the plant cell a first transfer cassette comprising, in operable linkage,
i) a polynucleotide construct comprising the polynucleotide of interest, wherein the polynucleotide construct is flanked by a first recombination site and a second non-identical recombination site; and
ii) a first recombinase construct encoding a first recombinase;
b) introducing into the plant cell a second transfer cassette comprising, in operable linkage;
i) a cell proliferation factor construct encoding a cell proliferation factor and a second recombinase construct encoding a second recombinase, wherein the cell proliferation factor construct and the second recombinase construct are flanked by a third recombination site and a fourth identical recombination site, wherein the third recombination site and the fourth identical recombination site are non-identical to the first recombination site and the second non-identical recombination site, and wherein the second recombinase is different than the first recombinase;
wherein the plant cell comprises the genomic target locus comprising the first recombination site and the second non-identical recombination site; whereby the first recombinase recognizes and implements recombination at the first recombination site and the second non-identical recombination site such that the polynucleotide construct is inserted at the target locus.

17. An agrobacterium -mediated transformation method for targeting the insertion of a polynucleotide of interest to a genomic target locus in a plant cell, said method comprising:
introducing into the plant cell a first transfer cassette and a second transfer cassette, each of the first transfer cassette and the second transfer cassette flanked by a right border and a left border and comprising between each right border and each left border, in operable linkage:
a) the first transfer cassette comprising a polynucleotide construct comprising the polynucleotide of interest, wherein the polynucleotide construct is flanked by a first recombination site and a second non-identical recombination site; and
b) the second transfer cassette comprising a cell proliferation factor construct encoding a cell proliferation factor, a first recombinase construct encoding a first recombinase, and a second recombinase construct encoding a second recombinase which is different from the first recombinase, wherein the cell proliferation factor construct and the first recombinase construct are flanked by a third recombination site and a fourth identical recombination site, wherein the third recombination site and the fourth identical recombination site are non-identical to the first recombination site and the second non-identical recombination site;
wherein the plant cell comprises the genomic target locus comprising the first recombination site and the second non-identical recombination site, whereby the first recombinase recognizes and implements recombination at the first recombination site and the second non-identical recombination site such that the polynucleotide construct is inserted at the target site.

18. An agrobacterium-mediated transformation method for targeting the insertion of a polynucleotide of interest to a genomic target locus in a plant cell, said method comprising:
introducing into the plant cell a first transfer cassette and a second transfer cassette, each of the first transfer cassette and the second transfer cassette flanked by a right border and a left border and comprising between each right border and each left border, in operable linkage:
a) the first transfer cassette comprising a polynucleotide construct comprising the polynucleotide of interest, wherein the polynucleotide construct is flanked by a first recombination site and a second non-identical recombination site;
b) a second transfer cassette comprising a cell proliferation factor construct encoding a cell proliferation factor, a first recombinase construct encoding a first recombinase, and a second recombinase construct encoding a second recombinase different than the first recombinase, wherein the cell proliferation factor construct, the first recombinase construct, and the second recombinase construct are flanked by a third recombination site and a fourth identical recombination site, wherein the third recombination site and the fourth identical recombination site are non-identical to the first recombination site and the second non-identical recombination site;
wherein the plant cell comprises the genomic target locus comprising the first recombination site and the second non-identical recombination site; whereby the first recombinase recognizes and implements recombination at the first recombination site and the second non-identical recombination site such that the polynucleotide construct is inserted at the target locus.

19. An agrobacterium-mediated transformation method for targeting the insertion of a polynucleotide of interest to a genomic target locus in a plant cell, said method comprising:
introducing into the plant cell a first transfer cassette and a second transfer cassette, each of the first transfer cassette and the second transfer cassette flanked by a right border and a left border and comprising between each right border and each left border, in operable linkage:
a) the first transfer cassette comprising a first recombination site adjacent to the right border and a second non-identical recombination site adjacent to the left border, a polynucleotide construct comprising the polynucleotide of interest, wherein the polynucleotide construct is flanked by the first recombination site and a third non-identical recombination site, and a recombinase construct encoding a recombinase and flanked by the third recombination site and a fourth recombination site identical to the third recombination site; and
b) the second transfer cassette comprising a cell proliferation factor construct encoding a cell proliferation factor, wherein the cell proliferation construct is flanked by a fifth recombination site and a sixth identical recombination site, wherein the fifth recombination site and the sixth identical recombination site are non-identical to the first recombination site and the second non-identical recombination site, and are identical to, or non-identical to, the third recombination site and the fourth identical recombination sites, and;
wherein the plant cell comprises the genomic target locus comprising the first recombination site and the second non-identical recombination site, whereby the recombinase recognizes and implements recombination at the first recombination site and the second non-identical recombination site such that the polynucleotide construct is inserted at the target locus.

20. An agrobacterium-mediated transformation method for targeting the insertion of a polynucleotide of interest to a genomic target locus in a plant cell, the method comprising:
a) introducing into the plant cell a first transfer cassette comprising, in operable linkage; a polynucleotide construct comprising the polynucleotide of interest, and a first recombinase construct encoding a first recombinase, wherein the polynucleotide construct and the first recombinase construct are flanked by a first recombination site and a second non-identical recombination site; and
b) introducing into the plant cell a second transfer cassette comprising, in operable linkage; a cell proliferation factor construct encoding a cell proliferation factor and a second recombinase construct encoding a second recombinase different from the first recombinase, wherein the cell proliferation factor construct and the second recombinase construct are flanked by a third recombination site and a fourth identical recombination site, wherein the third recombination site and the fourth recombination site are non-identical to the first recombination site and the second non-identical recombination sites;
wherein the plant cell comprises the genomic target locus comprising the first recombination site and the second non-identical recombination site; whereby the first recombinase recognizes and implements recombination at the first recombination site and the second non-identical recombination site such that the polynucleotide construct is inserted at the target site.

21. An agrobacterium-mediated transformation method for targeting the insertion of a polynucleotide of interest to a genomic target locus in a plant cell, the method comprising:
a) introducing into the plant cell a first transfer cassette comprising, in operable linkage;
i) a polynucleotide construct comprising the polynucleotide of interest and flanked by a first recombination site and a second non-identical recombination site; and
ii) a first recombinase construct encoding a first recombinase and flanked by a third recombination site and a fourth identical recombination site, wherein the third recombination site and the fourth identical recombination site are non-identical to the first recombination site and the second non-identical recombination site, and wherein the first recombinase construct and flanking third recombination site and fourth identical recombination site are inside the first recombination site and the second non-identical recombination site; and
b) introducing into the plant cell a second transfer cassette comprising, in operable linkage;
i) a cell proliferation factor construct encoding a cell proliferation factor, and a second recombinase construct encoding a second recombinase different than the first recombinase, wherein the cell proliferation factor construct and the second recombinase construct are flanked by the third recombination site and the fourth identical recombination site;
wherein the plant cell comprises the genomic target locus comprising the first recombination site and the second non-identical recombination site; whereby the first recombinase recognizes and implements recombination at the first recombination site and the second non-identical recombination site such that the polynucleotide construct is inserted at the target locus.

22. The method of any one of the preceding claims, the method further comprising selecting for integration events with or without the cell proliferation factor and the recombinase.

23. The method of any one of the preceding claims, wherein the polynucleotide of interest confers a trait selected from the group consisting of pest resistance, herbicide resistance, stress tolerance, drought tolerance, disease resistance, the ability to produce a chemical, and combinations thereof.

24. The method of any one of the preceding claims, wherein the first, the second, the third, the fourth, the fifth, and the sixth recombination site is selected from the group consisting of RS, gix, lox, FRT, rox, an integrase, an invertase, a resolvase, and a chimeric recombinase site.

25. The method of any one of the preceding claims, wherein one or more of the first, the second, the third, the fourth, the fifth, and the sixth recombination sites are FRT sites.

26. The method of any one of the preceding claims, wherein the first recombination site and the second non-identical sites are FRT sites.

27. The method of claim 26, wherein the non-identical first recombination and second sites are selected from the group consisting of FRT1/FRT6, FRT1/FRTS, FRT1/FRT12, and FRT1/FRT87.

28. The method of claim 27, wherein the non-identical sites are FRT1/FRT87.

29. The method of any one of the preceding claims, wherein one or more of the first, the second, the third, the fourth, the fifth, and the sixth recombination sites are lox sites.

30. The method of claim 29, wherein the lox sites are selected from the group consisting of loxP, lox 511, lox 5171, lox 2272, lox 71, lox 66, M2, M3, M7, and M11.

31. The method of claim 30, wherein the lox site is loxP.

32. The method of any one of the preceding claims, wherein the recombinase is selected from the group consisting of FLP, Cre, S-CRE, V-CRE, Dre, SSV1, lambda Int, phi C31 Int, HK022, R, Gin, Tn1721, CinH, ParA, Tn5053, Bxb1, TP907-1, and U153.

33. The method of any one of the preceding claims, wherein the recombinase is FLP.

34. The method of any one of the preceding claims, wherein the cell proliferation factor is selected from the group consisting of a Lec1 polypeptide, a Knotted1 (Kn1) polypeptide, a WUS/WOX homeobox polypeptide, a Zwille polypeptide, a babyboom (ODP2/BBM) polypeptide, an Aintegumenta polypeptide (ANT), a FUS3 polypeptide, a STM polypeptide, an OSH1 polypeptide, a SbH1 polypeptide, or any combination thereof.

35. The method of any one of the preceding claims, wherein the cell proliferation factor is selected from the group consisting of a WUS/WOX homeoboxL polypeptide, a babyboom polypeptide, and combinations thereof.

36. The method of any one of the preceding claims, wherein one or more construct further comprises a regulatory element.

37. The method of claim 36, wherein the regulatory element is selected from the group consisting of a promoter, an operator, an activator, an enhancer, a ribosomal binding site, an initiation codon, a terminator, and any combinations thereof.

38. The method of claim 37, wherein the promoter is selected from the group consisting of a non-inducible promoter, an inducible promoter, a constitutive promoter, a tissue-specific promoter, an organ-specific promoter, a developmentally-regulated promoter, a wound-inducible promoter, a pathogen-inducible promoter, a chemical-regulated promoter, a tissue-preferred promoter, and any combinations thereof.

39. The method of any one of the preceding claims, wherein one or more construct further comprises an ancillary sequence.

40. The method of claim 39, wherein the ancillary sequence is selected from the group consisting of a linker, an adapter, an intron, a restriction site, an insulator, a selectable marker, and any combinations thereof.

41. The method of any one of the preceding claims, wherein the plant cell is from a monocot.

42. The method of claim 41, wherein the monocot is selected from the group consisting of maize, rice, sorghum, wheat, sugarcane and millet.

43. The method of any one of the preceding claims, wherein the plant cell is from a dicot.

44. The method of claim 43, wherein the dicot is selected from the group consisting of canola, Brassica, soybean, sunflower, and cotton.

45. The method of any one of the preceding claims, wherein recombinase-mediated recombination results in about 2 or fewer integration events.
